SEQUENCE LISTING (49 sequences)

SEQ ID NO: 1 - protein, 216 aa, Artificial Sequence
  Description of Artificial Sequence Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 2 - protein, 450 aa, Artificial Sequence
  Description of Artificial Sequence Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 607 aa, Artificial Sequence
  Description of Artificial Sequence Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 4 - DNA, 711 bp, Artificial Sequence
  Description of Artificial Sequence Synthetic polynucleotide
atgagggccctgctggctagactgctgctgtgcgtgctggtcgtgtccgacagcaagggccagtccgccctgacccagcctgcctccgtgtctggctcccctggccagtccatcaccatcagctgcaccggcacctccagcgacgtgggcggctacaactacgtgtcctggtatcagcagcaccccggcaaggcccccaagctgatgatctacgacgtgtccaaccggccctccggcgtgtccaacagattctccggctccaagtccggcaacaccgcctccctgaccatcagcggactgcaggcagaggacgaggccgactactactgctcctcctacacctcctccagcaccagagtgttcggcaccggcacaaaagtgaccgtgctgggccagcccaaggccaacccaaccgtgacactgttccccccatcctccgaggaactgcaggccaacaaggccaccctggtctgcctgatctcagatttctatccaggcgccgtgaccgtggcctggaaggctgatggctccccagtgaaggccggcgtggaaaccaccaagccctccaagcagtccaacaacaaatacgccgcctcctcctacctgtccctgacccccgagcagtggaagtcccaccggtcctacagctgccaggtcacacacgagggctccaccgtggaaaagaccgtcgcccccaccgagtgctcatga

SEQ ID NO: 5 - DNA, 1887 bp, Artificial Sequence
  Description of Artificial Sequence Synthetic polynucleotide
atggaaacagacaccctgctgctgtgggtgctgctgctgtgggtgcccggctccacaggcgaggtgcagctgctggaatccggcggaggactggtgcagcctggcggctccctgagactgtcttgcgccgcctccggcttcaccttctccagctacatcatgatgtgggtgcgacaggcccctggcaagggcctggaatgggtgtcctccatctacccctccggcggcatcaccttctacgccgacaccgtgaagggccggttcaccatctcccgggacaactccaagaacaccctgtacctgcagatgaactccctgcgggccgaggacaccgccgtgtactactgcgcccggatcaagctgggcaccgtgaccaccgtggactactggggccagggcaccctggtgacagtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtgctggcggcggaggaagcggaggaggtggcagcggtggcggtggctccggcggaggtggctccggaatccctccccacgtgcagaagtccgtgaacaacgacatgatcgtgaccgacaacaacggcgccgtgaagttccctcagctgtgcaagttctgcgacgtgaggttcagcacctgcgacaaccagaagtcctgcatgagcaactgcagcatcacaagcatctgcgagaagccccaggaggtgtgtgtggccgtgtggaggaagaacgacgaaaacatcaccctcgagaccgtgtgccatgaccccaagctgccctaccacgacttcatcctggaagacgccgcctcccccaagtgcatcatgaaggagaagaagaagcccggcgagaccttcttcatgtgcagctgcagcagcgacgagtgcaatgacaacatcatctttagcgaggagtacaacaccagcaaccccgactgataa

SEQ ID NO: 6 - protein, 216 aa, Artificial Sequence
  Description of Artificial Sequence Synthetic polypeptide
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 7 - protein, 607 aa, Artificial Sequence
  Description of Artificial Sequence Synthetic polypeptide
EVQLLESGGGLVQPGGSLRLSCAASGFTFSMYMMMWVRQAPGKGLEWVSSIYPSGGITFYADSVKGRFTISRDNSKNTLYLQMNSLRAEDTAIYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 8 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 9 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 10 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 11 - protein, 21 aa, Artificial Sequence
  Description of Artificial Sequence Synthetic peptide
GGGGSGGGGSGGGGSGGGGSG

SEQ ID NO: 12 - protein, 118 aa, Artificial Sequence [sequence omitted]

SEQ ID NO: 13 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 14 - protein, 118 aa, Artificial Sequence
  Description of Artificial Sequence Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 15 - protein, 4 aa, Artificial Sequence
  Description of Artificial Sequence Synthetic peptide
QFNS

SEQ ID NO: 16 - protein, 4 aa, Artificial Sequence
  Description of Artificial Sequence Synthetic peptide
QAQS

SEQ ID NO: 17 - protein, 6 aa, Artificial Sequence
  Description of Artificial Sequence Synthetic peptide
PKSCDK

SEQ ID NO: 18 - protein, 6 aa, Artificial Sequence
  Description of Artificial Sequence Synthetic peptide
PKSSDK

SEQ ID NO: 19 - protein, 4 aa, Artificial Sequence
  Description of Artificial Sequence Synthetic peptide
LSLS

SEQ ID NO: 20 - protein, 4 aa, Artificial Sequence
  Description of Artificial Sequence Synthetic peptide
ATAT

SEQ ID NO: 21 - protein, 5 aa, Artificial Sequence
  Description of Artificial Sequence Synthetic peptide
  MOD_RES (1)..(1): Lys, Arg, Thr, Gln, Gly, Ala, Trp, Met, Ile or Ser
  MOD_RES (3)..(3): Val, Arg, Lys, Leu, Met or Ile
  MOD_RES (5)..(5): His, Thr, Asn, Gln, Ala, Val, Tyr, Trp, Phe or Met
XYXMX

SEQ ID NO: 22 - protein, 17 aa, Artificial Sequence
  Description of Artificial Sequence Synthetic peptide
  MOD_RES (8)..(8): Phe or Ile
  MOD_RES (14)..(14): Ser or Thr
SIYPSGGXTFYADXVKG

SEQ ID NO: 23 - protein, 11 aa, Artificial Sequence
  Description of Artificial Sequence Synthetic peptide
  MOD_RES (10)..(10): Glu or Asp
IKLGTVTTVXY

SEQ ID NO: 24 - protein, 30 aa, Artificial Sequence
  Description of Artificial Sequence Synthetic polypeptide
EVQLLESGGGLVQPGGSLRLSCAASGFTFS

SEQ ID NO: 25 - protein, 14 aa, Artificial Sequence
  Description of Artificial Sequence Synthetic peptide
WVRQAPGKGLEWVS

SEQ ID NO: 26 - protein, 32 aa, Artificial Sequence
  Description of Artificial Sequence Synthetic polypeptide
RFTISRDNSKNTLYLQMNSLRAEDTAVYYCAR

SEQ ID NO: 27 - protein, 11 aa, Artificial Sequence
  Description of Artificial Sequence Synthetic peptide
WGQGTLVTVSS

SEQ ID NO: 28 - protein, 14 aa, Artificial Sequence
  Description of Artificial Sequence Synthetic peptide
  MOD_RES (4)..(4): Asn or Ser
  MOD_RES (5)..(5): Thr, Arg or Ser
  MOD_RES (9)..(9): Ala or Gly
TGTXXDVGXYNYVS

SEQ ID NO: 29 - protein, 7 aa, Artificial Sequence
  Description of Artificial Sequence Synthetic peptide
  MOD_RES (1)..(1): Glu or Asp
  MOD_RES (3)..(3): Ile, Asn or Ser
  MOD_RES (4)..(4): Asp, His or Asn
XVXXRPS

SEQ ID NO: 30 - protein, 10 aa, Artificial Sequence
  Description of Artificial Sequence Synthetic peptide
  MOD_RES (3)..(3): Phe or Tyr
  MOD_RES (5)..(5): Asn or Ser
  MOD_RES (6)..(6): Arg, Thr or Ser
  MOD_RES (7)..(7): Gly or Ser
  MOD_RES (8)..(8): Ile or Thr
SSXTXXXXRV

SEQ ID NO: 31 - protein, 22 aa, Artificial Sequence
  Description of Artificial Sequence Synthetic peptide
QSALTQPASVSGSPGQSITISC

SEQ ID NO: 32 - protein, 15 aa, Artificial Sequence
  Description of Artificial Sequence Synthetic peptide
WYQQHPGKAPKLMIY

SEQ ID NO: 33 - protein, 32 aa, Artificial Sequence
  Description of Artificial Sequence Synthetic polypeptide
GVSNRFSGSKSGNTASLTISGLQAEDEADYYC

SEQ ID NO: 34 - protein, 10 aa, Artificial Sequence
  Description of Artificial Sequence Synthetic peptide
FGTGTKVTVL

SEQ ID NO: 35 - protein, 5 aa, Artificial Sequence
  Description of Artificial Sequence Synthetic peptide
SYIMM

SEQ ID NO: 36 - protein, 17 aa, Artificial Sequence
  Description of Artificial Sequence Synthetic peptide
SIYPSGGITFYADTVKG

SEQ ID NO: 37 - protein, 11 aa, Artificial Sequence
  Description of Artificial Sequence Synthetic peptide
IKLGTVTTVDY

SEQ ID NO: 38 - protein, 14 aa, Artificial Sequence
  Description of Artificial Sequence Synthetic peptide
TGTSSDVGGYNYVS

SEQ ID NO: 39 - protein, 7 aa, Artificial Sequence
  Description of Artificial Sequence Synthetic peptide
DVSNRPS

SEQ ID NO: 40 - protein, 10 aa, Artificial Sequence
  Description of Artificial Sequence Synthetic peptide
SSYTSSSTRV

SEQ ID NO: 41 - protein, 5 aa, Artificial Sequence
  Description of Artificial Sequence Synthetic peptide
MYMMM

SEQ ID NO: 42 - protein, 17 aa, Artificial Sequence
  Description of Artificial Sequence Synthetic peptide
SIYPSGGITFYADSVKG

SEQ ID NO: 43 - protein, 14 aa, Artificial Sequence
  Description of Artificial Sequence Synthetic peptide
TGTSSDVGAYNYVS

SEQ ID NO: 44 - protein, 119 aa, Artificial Sequence
  Description of Artificial Sequence Synthetic polypeptide
EVQLLESGGGLVQPGGSLRLSCAASGFTFSSYIMMVWRQAPGKGLEWVSSIYPSGGITFYADWKGRFTISRDNSKNTLYLQMNSLRAEDTAVYYCARIKLGTVTTVDYWGQGTLVTVSS

SEQ ID NO: 45 - protein, 110 aa, Artificial Sequence
  Description of Artificial Sequence Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 46 - protein, 120 aa, Artificial Sequence
  Description of Artificial Sequence Synthetic polypeptide
EVQLLESGGGLVQPGGSLRLSCAASGFTFSMYMMMWVRQAPGKGLEVWSSIYPSGGITFYADSVKGRFTISRDNSKNTLYLQMNSLRAEDTAIYYCARIKLGTVTTVDYWGQGTLVTVSS

SEQ ID NO: 47 - protein, 110 aa, Artificial Sequence
  Description of Artificial Sequence Synthetic polypeptide
QSALTQPASVSGSPGQSITISCTGTSSDVGAYNYVSWYQQHPGKAPKLMIYDVSNRPSGVSNRFSGSKSGNTASLTISGLQAEDEADYYCSSYTSSSTRVFGTGTKVTVL

SEQ ID NO: 48 - DNA, 1407 bp, Artificial Sequence
  Description of Artificial Sequence Synthetic polynucleotide from human Fab library
atggagttgcctgttaggctgttggtgctgatgttctggattcctgctagctccagcgaggtgcagctgctggaatccggcggaggactggtgcagcctggcggctccctgagactgtcttgcgccgcctccggcttcaccttctccagctacatcatgatgtgggtgcgacaggcccctggcaagggcctggaatgggtgtcctccatctacccctccggcggcatcaccttctacgccgacaccgtgaagggccggttcaccatctcccgggacaactccaagaacaccctgtacctgcagatgaactccctgcgggccgaggacaccgccgtgtactactgcgcccggatcaagctgggcaccgtgaccaccgtggactactggggccagggcaccctggtgacagtgtcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 49 - DNA, 705 bp, Artificial Sequence
  Description of Artificial Sequence Synthetic polynucleotide from human Fab library
atggagttgcctgttaggctgttggtgctgatgttctggattcctgcttccttaagccagtccgccctgacccagcctgcctccgtgtctggctcccctggccagtccatcaccatcagctgcaccggcacctccagcgacgtgggcggctacaactacgtgtcctggtatcagcagcaccccggcaaggcccccaagctgatgatctacgacgtgtccaaccggccctccggcgtgtccaacagattctccggctccaagtccggcaacaccgcctccctgaccatcagcggactgcaggcagaggacgaggccgactactactgctcctcctacacctcctccagcaccagagtgttcggcaccggcacaaaagtgaccgtgctgggccagcccaaggccaacccaaccgtgacactgttccccccatcctccgaggaactgcaggccaacaaggccaccctggtctgcctgatctcagatttctatccaggcgccgtgaccgtggcctggaaggctgatggctccccagtgaaggccggcgtggaaaccaccaagccctccaagcagtccaacaacaaatacgccgcctcctcctacctgtccctgacccccgagcagtggaagtcccaccggtcctacagctgccaggtcacacacgagggctccaccgtggaaaagaccgtcgcccccaccgagtgctca

(19) United States
(12) Patent Application Publication
(10) Pub. No.: US 2019/0330375 A1
(43) Pub. Date: Oct. 31, 2019

(54) DOSING REGIMENS AND DOSAGE FORMS FOR TARGETED TGF-B INHIBITION

(71) Applicant: Merck Patent GmbH, Darmstadt (DE)

(72) Inventors: Samer El Bawab, Frankfurt Am Main (DE); Isabelle Dussault, Needham, MA (US); Yulia Vugmeyster, Winchester, MA (US); Akash Khandelwal, Griesheim (DE)

(21) Appl. No.: 16/460,792

(22) Filed: Jul. 2, 2019

Related U.S. Application Data
(63) Continuation of application No. PCT/US2018/012604, filed on Jan. 5, 2018.
(60) Provisional application No. 62/581,978, filed on Nov. 6, 2017.
(60) Provisional application No. 62/443,698, filed on Jan. 7, 2017.

(51) Int. Cl. C07K 16/46; C07K 16/28; C07K 14/71; A61K 9/00; A61J 1/10; A61P 35/00

(52) U.S. Cl. CPC C07K 16/468; C07K 16/2827; C07K 14/71; A61K 2039/545; A61J 1/10; A61P 35/00; A61K 9/0019

(57) ABSTRACT

This disclosure relates generally to body weight independent (BW-independent) dosing regimens and dosage forms of a bifunctional protein targeting human protein Programmed Death Ligand 1 (PD-L1) and Transforming Growth Factor β (TGFβ).

### CROSS-REFERENCE TO RELATED APPLICATIONS

This application claims the benefit of and priority to U.S. application No. 62/443,698, filed Jan. 7, 2017, and U.S. application No. 62/581,978, filed Nov. 6, 2017, contents of each of which are hereby incorporated by reference in their entireties for all purposes.

### FIELD OF THE DISCLOSURE

The present disclosure relates generally to body weight independent (BW-independent) dosing regimens and dosage forms of a bifunctional protein targeting human protein Programmed Death Ligand 1 (PD-L1) and Transforming Growth Factor β (TGFβ).

### BACKGROUND

The programmed death 1 (PD-1)/PD-L1 axis is an important mechanism for tumor immune evasion. Effector T cells chronically sensing antigen take on an exhausted phenotype marked by PD-1 expression, a state under which tumor cells engage by upregulating PD-L1. Additionally, in the tumor microenvironment, myeloid cells, macrophages, parenchymal cells and T cells upregulate PD-L1. Blocking the axis restores the effector function in these T cells.

US patent application publication number US 20150225483 A1, incorporated herein by reference, describes a bi-functional fusion protein that combines an anti-programmed death ligand 1 (PD-L1) antibody with the soluble extracellular domain of tumor growth factor beta receptor type II (TGFβRII) as a TGFβ neutralizing “Trap,” into a single molecule. Specifically, the protein is a heterotetramer, consisting of the two immunoglobulin light chains of anti-PD-L1, and two heavy chains comprising the heavy chain of anti-PD-L1 genetically fused via a flexible glycine-serine linker to the extracellular domain of the human TGFβRII (see FIG. 1). This anti-PD-L1/TGFβ Trap molecule is designed to target two major mechanisms of immunosuppression in the tumor microenvironment. US patent application publication number US 20150225483 A1 describes administration of the Trap molecule at doses based on the patient's weight.

### SUMMARY OF THE DISCLOSURE

The present disclosure provides improved dosing regimens for administration of bifunctional proteins targeting PD-L1 and TGFβ. Specifically, body weight independent (BW-independent) dosing regimens and related dosage forms involving administration of at least 500 mg of the bifunctional protein administered at various dosing frequencies can be used as an anti-tumor and anti-cancer therapeutic. The BW-independent dosing regimen ensures that all patients, irrespective of their body weight, will have adequate drug exposure at the tumor site.

The bifunctional protein of the present disclosure (anti-PD-L1/TGFβ Trap molecule) includes a first and a second polypeptide. The first polypeptide includes: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ) (e.g., a soluble fragment). The second polypeptide includes at least a variable region of a light chain of an antibody that binds PD-L1, in which the heavy chain of the first polypeptide and the light chain of the second polypeptide, when combined, form an antigen binding site that binds PD-L1 (e.g., any of the antibodies or antibody fragments described herein). Because the bifunctional protein of the present disclosure binds to two targets, (1) PD-L1, which is largely membrane bound, and (2) TGFβ, which is soluble in blood and interstitium, the BW-independent dosing regimen requires a dose that is effective not only to inhibit PD-L1 at the tumor site but also sufficient to inhibit TGFβ.

In one aspect, the disclosure provides treatment of a cancer or inhibition of a tumor, e.g., non-small cell lung cancer, melanoma, pancreatic cancer, colorectal cancer (e.g., pretreated colorectal cancer (CRC)), ovarian cancer, glioblastoma, gastric cancer (e.g., pretreated recurrent or refractory unresectable Stage IV gastric cancer), biliary tract cancer, esophageal cancer (squamous cell carcinoma or adenocarcinoma), adenoma of the head or the neck, and squamous carcinoma of the head or the neck.

The disclosure also features a bifunctional protein described above for use in treating cancer or for use in inhibiting tumor growth. The cancer or tumor may be selected from colorectal (e.g., pretreated colorectal cancer (CRC)), breast, ovarian, pancreatic, gastric (e.g., pretreated recurrent or refractory unresectable Stage IV gastric cancer), prostate, renal, cervical, myeloma, lymphoma, leukemia, thyroid, endometrial, uterine, bladder, neuroendocrine, head and neck, liver, nasopharyngeal, testicular, small cell lung cancer, non-small cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodysplastic syndromes. The use may further include administration of radiation or administration of a chemotherapeutic, a biologic, or a vaccine.

The disclosure also features a method of promoting local depletion of TGFβ. The method includes administering a protein described above, where the protein binds TGFβ in solution, binds PD-L1 on a cell surface, and carries the bound TGFβ into the cell (e.g., a cancer cell).

The disclosure also features a method of inhibiting SMAD3 phosphorylation in a cell (e.g., a cancer cell or an immune cell), the method including exposing the cell in the tumor microenvironment to a protein described above.

The disclosure also features a method of inhibiting tumor growth or treating cancer. The method includes exposing the tumor to a protein described above. The method may further include exposing the tumor to radiation or to a chemotherapeutic, a biologic, or a vaccine. In certain embodiments, the tumor or cancer is selected from colorectal (e.g., pretreated colorectal cancer (CRC)), breast, ovarian, pancreatic, gastric (e.g., pretreated recurrent or refractory unresectable Stage IV gastric cancer), prostate, renal, cervical, myeloma, lymphoma, leukemia, thyroid, endometrial, uterine, bladder, neuroendocrine, head and neck, liver, nasopharyngeal, testicular, small cell lung cancer, non-small cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodysplastic syndromes.

By “TGFβRII” or “TGFβ Receptor II” is meant a polypeptide having the wild-type human TGFβ Receptor Type 2 Isoform A sequence (e.g., the amino acid sequence of NCBI Reference Sequence (RefSeq) Accession No. NP_001020018 (SEQ ID NO. 8)), or a polypeptide having the wild-type human TGFβ Receptor Type 2 Isoform B sequence (e.g., the amino acid sequence of NCBI RefSeq Accession No. NP_003233 (SEQ ID NO. 9)) or having a sequence substantially identical the amino acid sequence of SEQ ID NO. 8 or of SEQ ID NO. 9. The TGFβRII may retain at least 0.1%, 0.5%, 1%, 5%, 10%, 25%, 35%, 50%, 75%, 90%, 95%, or 99% of the TGFβ-binding activity of the wild-type sequence. The polypeptide of expressed TGFβRII lacks the signal sequence.

By a “fragment of TGFβRII capable of binding TGFβ” is meant any portion of NCBI RefSeq Accession No. NP_001020018 (SEQ ID NO. 8) or of NCBI RefSeq Accession No. NP_003233 (SEQ ID NO. 9), or a sequence substantially identical to SEQ ID NO. 8 or SEQ ID NO. 9 that is at least 20 (e.g., at least 30, 40, 50, 60, 70, 80, 90, 100, 110, 120, 130, 140, 150, 160, 175, or 200) amino acids in length that retains at least some of the TGFβ-binding activity (e.g., at least 0.1%, 0.5%, 1%, 5%, 10%, 25%, 35%, 50%, 75%, 90%, 95%, or 99%) of the wild-type receptor or of the corresponding wild-type fragment. Typically such fragment is a soluble fragment. An exemplary such fragment is a TGFβRII extra-cellular domain having the sequence of SEQ ID NO: 10.

By “substantially identical” is meant a polypeptide exhibiting at least 50%, desirably 60%, 70%, 75%, or 80%, more desirably 85%, 90%, or 95%, and most desirably 99% amino acid sequence identity to a reference amino acid sequence. The length of comparison sequences will generally be at least 10 amino acids, desirably at least 15 contiguous amino acids, more desirably at least 20, 25, 50, 75, 90, 100, 150, 200, 250, 300, or 350 contiguous amino acids, and most desirably the full-length amino acid sequence.

By “patient” is meant either a human or non-human animal (e.g., a mammal). “Patient,” “subject,” “patient in need thereof,” and “subject in need thereof” are used interchangeably in this disclosure, and refer to a living organism suffering from or prone to a disease or condition that can be treated by administration using the methods and compositions provided in this disclosure.

The terms “treat,” “treating,” or “treatment,” and other grammatical equivalents as used in this disclosure, include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

By “cancer” is meant a collection of cells multiplying in an abnormal manner. As used herein, the term “cancer” refers to all types of cancer, neoplasm, malignant or benign tumors found in mammals, including leukemia, carcinomas, and sarcomas. Exemplary cancers include breast cancer, ovarian cancer, colon cancer, liver cancer, kidney cancer, lung cancer, pancreatic cancer, glioblastoma. Additional examples include cancer of the brain, lung cancer, non-small cell lung cancer, melanoma, sarcomas, prostate cancer, cervix cancer, stomach cancer, head and neck cancers, uterus cancer, mesothelioma, metastatic bone cancer, medulloblastoma, Hodgkin's Disease, Non-Hodgkin's Lymphoma, multiple myeloma, neuroblastoma, rhabdomyosarcoma, primary thrombocytosis, primary macrobulinemia, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, and neoplasms of the endocrine and exocrine pancreas.

Throughout the description and claims of this specification the word “comprise” and other forms of the word, such as “comprising” and “comprises,” means including but not limited to, and is not intended to exclude, for example, other components.

By “co-administer” it is meant that a composition described herein is administered at the same time, just prior to, or just after the administration of additional therapies. The protein and the composition of the present disclosure can be administered alone or can be co-administered with a second, third, or fourth therapeutic agent(s) to a patient. Co-administration is meant to include simultaneous or sequential administration of the protein or composition individually or in combination (more than one therapeutic agent).

The term “a” is not meant to limit as a singular. In certain embodiments, the term “a” may refer to a plural form. As used throughout this disclosure, the singular forms “a,” “an,” and “the” include plural reference unless the context clearly dictates otherwise. Thus, for example, a reference to “a composition” includes a plurality of such compositions, as well as a single composition.

A “reconstituted” formulation is one which has been prepared by dissolving a lyophilized formulation in an aqueous carrier such that the bifunctional molecule is dissolved in the reconstituted formulation. The reconstituted formulation is suitable for intravenous administration (IV) to a patient in need thereof.

The term “about” refers to any minimal alteration in the concentration or amount of an agent that does not change the efficacy of the agent in preparation of a formulation and in treatment of a disease or disorder. In embodiments, the term “about” may include ±15% of a specified numerical value or data point.

Ranges can be expressed in this disclosure as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed in this disclosure, and that each value is also disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

An “isotonic” formulation is one which has essentially the same osmotic pressure as human blood. Isotonic formulations will generally have an osmotic pressure from about 250 to 350 mOsmol/KgH₂O. The term “hypertonic” is used to describe a formulation with an osmotic pressure above that of human blood. Isotonicity can be measured using a vapor pressure or ice-freezing type osmometer, for example.

The term “buffering agent” refers to one or more components that when added to an aqueous solution is able to protect the solution against variations in pH when adding acid or alkali, or upon dilution with a solvent. In addition to phosphate buffers, there can be used glycinate, carbonate, citrate buffers and the like, in which case, sodium, potassium or ammonium ions can serve as counterion.

An “acid” is a substance that yields hydrogen ions in aqueous solution. A “pharmaceutically acceptable acid” includes inorganic and organic acids which are nontoxic at the concentration and manner in which they are formulated.

A “base” is a substance that yields hydroxyl ions in aqueous solution. “Pharmaceutically acceptable bases” include inorganic and organic bases which are non-toxic at the concentration and manner in which they are formulated.

A “lyoprotectant” is a molecule which, when combined with a protein of interest, prevents or reduces chemical and/or physical instability of the protein upon lyophilization and subsequent storage.

A “preservative” is an agent that reduces bacterial action and may be optionally added to the formulations herein. The addition of a preservative may, for example, facilitate the production of a multi-use (multiple-dose) formulation. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3pentanol, and m-cresol.

A “surfactant” is a surface active molecule containing both a hydrophobic portion (e.g., alkyl chain) and a hydrophilic portion (e.g., carboxyl and carboxylate groups). Surfactant may be added to the formulations of the invention. Surfactants suitable for use in the formulations of the present invention include, but are not limited to, polysorbates (e.g. polysorbates 20 or 80); poloxamers (e.g. poloxamer 188); sorbitan esters and derivatives; Triton; sodium laurel sulfate; sodium octyl glycoside; lauryl-, myristyl-, linoleyl-, or stearyl-sulfobetadine; lauryl-, myristyl-, linoleyl- or stearyl-sarcosine; linoleyl-, myristyl-, or cetyl-betaine; lauramidopropyl-cocamidopropyl-, linoleamidopropyl-, myristamidopropyl-, palmidopropyl-, or isostearamidopropylbetaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethylene glycol, polypropyl glycol, and copolymers of ethylene and propylene glycol (e.g., Pluronics, PF68 etc.).

Other embodiments and details of the disclosure are presented herein below.

### BRIEF DESCRIPTION OF THE DRAWINGS

FIG. 1 is a schematic drawing of an anti-PD-L1/TGFβ Trap molecule including one anti-PD-L1 antibody fused to two extracellular domain (ECD) of TGFβ Receptor II via a (Gly₄Ser)₄Gly (SEQ ID NO: 11) linker.

FIG. 2 shows a graph of a two-step ELISA demonstrating that anti-PD-L1/TGFβ Trap simultaneously binds to both PD-L1 and TGFβ.

FIG. 3 is a graph showing anti-PD-L1/TGFβ Trap induces a dramatic increase in IL-2 levels.

FIG. 4A is a graph showing in vivo depletion of TGFβ1 in response to the anti-PD-L1/TGFβ Trap. Line graphs represent naïve, isotype control, and three different doses, as indicated in the legend. FIG. 4B is a graph showing in vivo depletion of TGFβ2 in response to the anti-PD-L1/TGFβ Trap. Line graphs represent naïve, isotype control, and three different doses, as indicated in the legend. FIG. 4C is a graph showing in vivo depletion of TGFβ3 in response to the anti-PD-L1/TGFβ Trap. Line graphs represent naïve, isotype control, and three different doses, as indicated in the legend. FIG. 4D is a graph showing that occupancy of PD-L1 by the anti-PD-L1/TGFβ Trap supports a receptor binding model in the EMT-6 tumor system.

FIG. 5 is a graph showing anti-tumor efficacy of anti-PD-L1/TGFβ Trap control (anti-PD-L1(mut)/TGFβ) in Detroit 562 xenograft model.

FIG. 6A is a scatter-plot showing relationship between clearance and body weight. The line represents the regression line demonstrating relationship between CL and BW. FIG. 6B is a scatter-plot showing relationship between volume of distribution (V) and body weight. The line represents the regression line demonstrating relationship between V and BW.

FIG. 7A is a box-plot of C_{avg} distribution for an entire population for a fixed (1200 mg) versus mg/kg based dosing (17.65 mg/kg) in a simulated population of 68 kg median body weight. FIG. 7B is a box-plot of exposure AUC distribution for an entire population for a fixed (1200 mg) versus mg/kg based dosing (17.65 mg/kg) in a simulated population of 68 kg median body weight. FIG. 7C is a box-plot of C_{trough} distribution for an entire population for a fixed (1200 mg) versus mg/kg based dosing (17.65 mg/kg) in a simulated population of 68 kg median body weight. FIG. 7D is a box-plot of Cₘₐₓ distribution for an entire population for a fixed (1200 mg) versus mg/kg based dosing (17.65 mg/kg) in a simulated population of 68 kg median body weight.

FIG. 7E is a box-plot of C_{avg} distribution for an entire population for a fixed (500 mg) versus mg/kg based dosing (7.35 mg/kg) in a simulated population of 68 kg median body weight. FIG. 7F is a box-plot of exposure AUC distribution for an entire population for a fixed (500 mg) versus mg/kg based dosing (7.35 mg/kg) in a simulated population of 68 kg median body weight. FIG. 7G is a box-plot of C_{trough} distribution for an entire population for a fixed (500 mg) versus mg/kg based dosing (7.35 mg/kg) in a simulated population of 68 kg median body weight. FIG. 7H is a box-plot of Cₘₐₓ distribution for an entire population for a fixed (500 mg) versus mg/kg based dosing (7.35 mg/kg) in a simulated population of 68 kg median body weight.

FIG. 8A is a box-plot of C_{avg} distribution across body weight quartiles for a fixed (1200 mg) versus mg/kg (17.65 mg/kg) based dosing in a simulated population of 68 kg median body weight. FIG. 8B is a box-plot of exposure (AUC) distribution across body weight quartiles for a fixed (1200 mg) versus mg/kg (17.65 mg/kg) based dosing in a simulated population of 68 kg median body weight. FIG. 8C is a box-plot of C_{trough} distribution across body weight quartiles for a fixed (1200 mg) versus mg/kg (17.65 mg/kg) based dosing in a simulated population of 68 kg median body weight. FIG. 8D is a box-plot of Cₘₐₓ distribution across body weight quartiles for a fixed (1200 mg) versus mg/kg (17.65 mg/kg) based dosing in a simulated population of 68 kg median body weight.

FIG. 8E is a box-plot of C_{avg} distribution across body weight quartiles for a fixed (500 mg) versus mg/kg (7.35 mg/kg) based dosing in a simulated population of 68 kg median body weight. FIG. 8F is a box-plot of exposure (AUC) distribution across body weight quartiles for a fixed (500 mg) versus mg/kg (7.35 mg/kg) based dosing in a simulated population of 68 kg median body weight. FIG. 8G is a box-plot of C_{trough} distribution across body weight quartiles for a fixed (500 mg) versus mg/kg (7.35 mg/kg) based dosing in a simulated population of 68 kg median body weight. FIG. 8H is a box-plot of Cₘₐₓ distribution across body weight quartiles for a fixed (500 mg) versus mg/kg (7.35 mg/kg) based dosing in a simulated population of 68 kg median body weight.

FIG. 9A is a goodness of fit scatter plot for the PK-Efficacy model showing the predicted tumor volume vs. the observed tumor volume. FIG. 9B is a goodness of fit scatter plot for the PK-Efficacy model showing the conditional weighted residuals (GWRES) vs. the time after dose.

FIGS. 10A-10C are graphs showing the predicted PK and PD-L1 receptor occupancy (“RO”) of anti-PD-L1/TGFβ Trap molecules at doses and schedules associated with tumor regression in mice. FIG. 10A is a graph showing the predicted plasma concentration vs. time. FIG. 10B is a graph showing the predicted PD-L1 RO vs. time in PBMC. FIG. 10C is a graph showing the predicted PD-L1 RO vs. time in tumor.

FIGS. 11A-11C are graphs showing the predicted PK and PD-L1 receptor occupancy (“RO”) of ant-PD-L1/TGFβ Trap molecules at doses and schedules associated with tumor stasis in mice. FIG. 11A is a graph showing the predicted plasma concentration vs. time. FIG. 11B is a graph showing the predicted PD-L1 RO vs. time in PBMC. FIG. 11C is a graph showing the predicted PD-L1 RO vs. time in tumor.

FIGS. 12A-12B are box-plots of simulated exposure distribution (FIG. 12A: C_{overage}, FIG. 12B: C_{trough}) for entire population for various dosing regimens in a simulated population of 68 kg median body weight.

FIG. 13 is a spider plot that demonstrates that patients with previously progressive disease (both with primary refractory and acquired resistant disease) achieved significant disease stabilization. Patients with disease response and disease stabilization were noted to have a range in prior treatments prior to initiating this study, and even had a range of treatments immediately prior to starting on trial, suggesting clinical activity of anti-PD-L1/TGFβ Trap in a heterogeneous population of patients with prior PDx exposure (filled triangle: subject off-treatment; filled diamond: first occurrence of new lesion).

FIG. 14 shows a histogram of efficacy of an anti-PD-L1/TGFβ Trap molecule in patients treated with anti-PD-1/PD-L1 treatment. Efficacy of the anti-PD-L1/TGFβ Trap molecule was observed in some patients identified as refractory (black bars) and resistant (white bars) to prior anti-PD-1/PD-L1 population (a value of around zero (0) or a negative value of the percentage change in sum of diameters indicates efficacy).

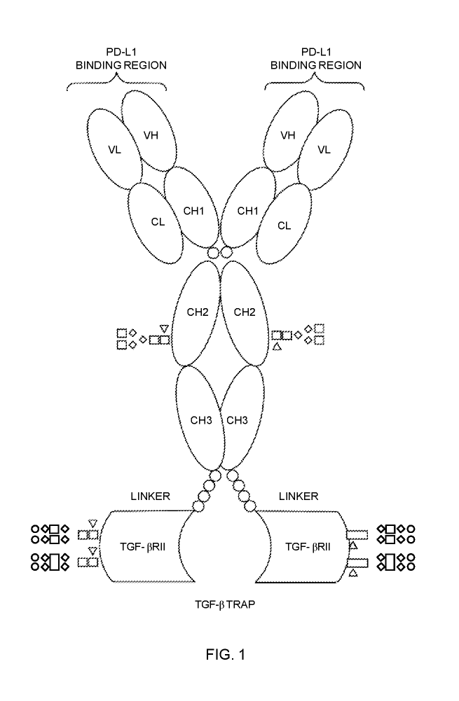

### DETAILED DESCRIPTION

### Body Weight-Independent Dosing Regimen

Body weight-independent dosing regimens involving the administration of at least 500 mg of the bifunctional anti-PD-L1/TGFβ Trap molecules described herein have been developed, informed by the results of a variety of pre-clinical and clinical assessments of the molecules. Two studies investigated the safety, tolerability, and pharmacokinetics of the molecules, and included assessments of PD-L1 target occupancy on peripheral blood mononuclear cells obtained from the blood of treated patients and measurements of the concentrations of TGFβ1, TGFβ2, and TGFβ3. These assessments were based on data from a total of 350 subjects (dose escalation cohorts of 1, 3, 10 and 20 mg/kg in solid tumors, and expansion cohorts of 3 mg/kg, 10 mg/kg, 500 mg, and 1200 mg in selected tumor types). As of the data cut-off date at the time of analysis, the median treatment duration was approximately 28 days.

### PK/Efficacy Model (Mouse Model)

Experiments were also conducted to determine the efficacy of the anti-PD-L1/TGFβ Trap molecule in a tumor model. Efficacy results from EMT-6 xenografts were used to establish the PK/Efficacy model. The established PK model in mice was used to simulate anti-PD-L1/TGFβ Trap plasma exposure for the efficacy experiment settings. The estimated parameters are reported in Table 1. The estimated KC50 value was 55.3 μg/mL. This value represents the average plasma concentrations for which 50% if the maximal anti-tumor activity of the anti-PD-L1/TGFβ Trap molecule could be achieved.

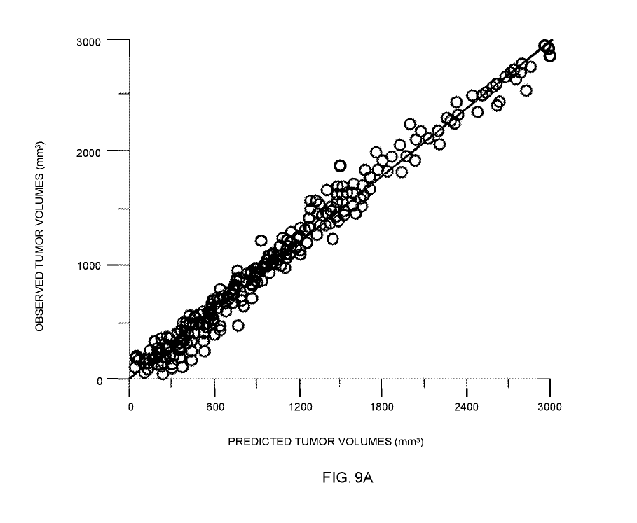

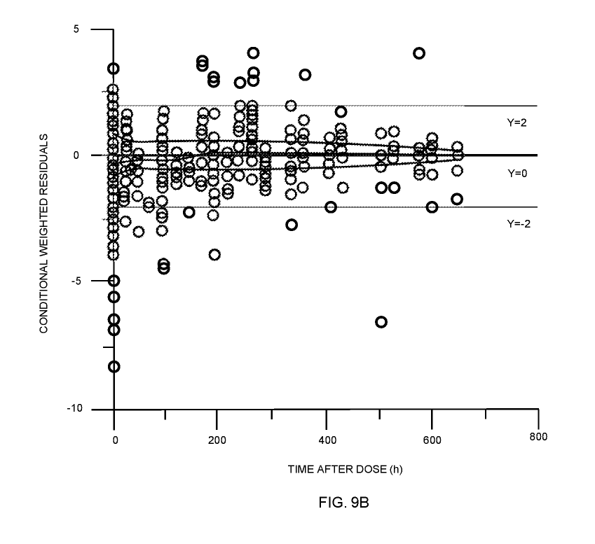

Basic diagnostics plots of the model revealed no model misspecification. The model predictions are able to capture the tumor volume distributions (FIG. 9A). Conditional weighted residuals are normally distributed with a 0 mean and 1 variance without a trend (FIG. 9B). The PK/Efficacy model was then used to simulate tumor growth inhibition (TGI) using the human predicted concentration-time profiles at different doses.

TABLE 1
Mouse PK/Efficacy model parameters for anti-PD-L1/TGFβ
Trap molecule in EMT-6 xenograft mice

| Parameters | Estimate | Std | CV % | % IIV |
|---|---|---|---|---|
| K_{g} (h⁻¹) | 0.068 | 0.0005 | 0.82 | 40 |
| Kₜᵣ (h⁻¹) | 0.055 | 0.0024 | 4.4 | 76 |
| KC₅₀ (ng/mL) | 55324.6 | 522.3 | 4.4 | 232 |
| Kₘₐₓ | 2 | 0.09 | 1 | 93 |
| Baseline (mm³) | 88.3 | 0.87 | 1 | 47 |

### Response Analysis Based on PD-L1 Occupancy (in a Mouse Model)

Using the efficacy experiments, responses in mice have been analyzed and sorted by either tumor regression or tumor stasis, and PK and PD-L1 receptor occupancy (RO) have been predicted based on the integrated PK/RO model. The approach demonstrated that an anti-PD-L1/TGFβ Trap molecule plasma concentration between 40 and 100 μg/mL associated with a PD-L1 RO above 95% in tumor is required to reach tumor regression (FIGS. 9A-9B). The plasma concentration of anti-PD-L1/TGFβ Trap molecule between 10 and 40 μg/mL associated with a PD-L1 RO above 95% in periphery is required to reach tumor stasis (FIGS. 10A-10C).

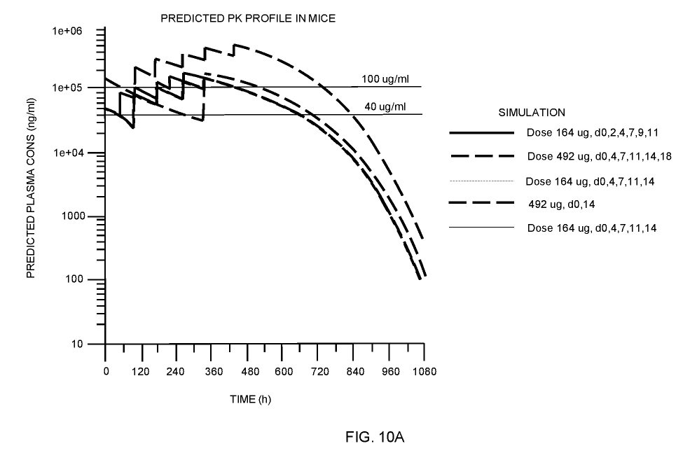

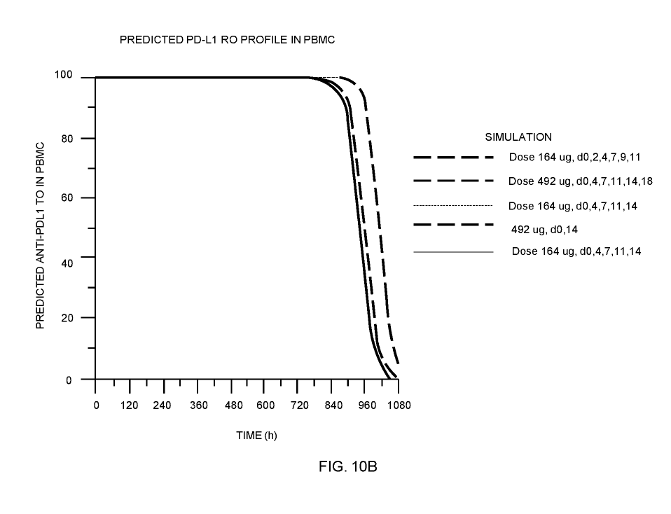

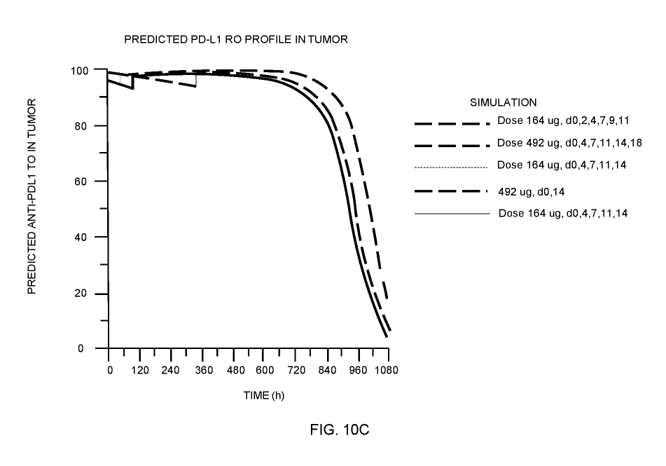

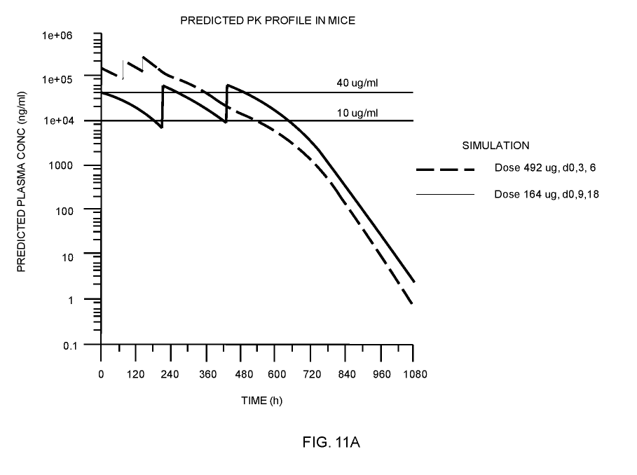

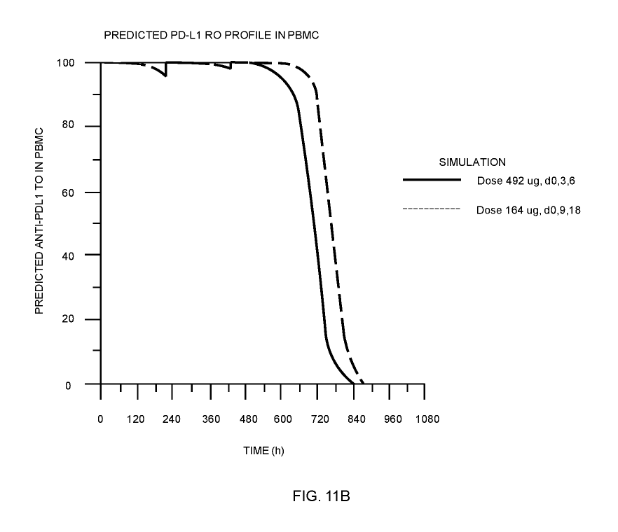

Response analysis and predicted PK/RO in mice lead to FIGS. 11A-11C, which summarize the PK/RO/Efficacy for the anti-PD-L1/TGFβ Trap molecule in mice. 95% of PD-L1 RO is achieved at a plasma concentration of 40 μg/mL with an expected/estimate TGI of only about 65%. Increasing the concentration above 40 μg/mL results in an additional increase in tumor growth inhibition. 95% of tumor growth inhibition is achieved at average plasma concentration of about 100 μg/mL.

Based on the population PK model described below, a flat dose of at least 500 mg administered once every two weeks is required to maintain an average concentration of about 100 μg/mL, while a flat dose of about 1200 mg administered once every two weeks is required to maintain a C_{trough} of about 100 μg/mL. In certain embodiments about 1200 mg to about 3000 mg (e.g., about 1200, about 1300, about 1400, about 1500, about 1600, about 1700, about 1800, about 1900, about 2000, about 2100, about 2200, about 2300, about 2400, etc.) of a protein product of the present disclosure (e.g., anti-PD-L1/TGFβ Trap) is administered to a subject. In certain embodiments, about 1200 mg of anti-PD-L1/TGFβ Trap molecule is administered to a subject once every two weeks. In certain embodiments, about 1800 mg of anti-PD-L1/TGFβ Trap molecule is administered to a subject once every three weeks.

In embodiments, about 1200 mg to about 3000 mg (e.g., about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, etc.) of the protein product with a first polypeptide that includes the amino acid sequence of SEQ ID NO: 3, and the second polypeptide that includes the amino acid sequence of SEQ ID NO: 1 is administered to the subject.

In certain embodiments, about 1200 mg of the protein product with a first polypeptide that includes the amino acid sequence of SEQ ID NO: 3, and the second polypeptide that includes the amino acid sequence of SEQ ID NO: 1 is administered to a subject once every two weeks. In certain embodiments, about 1800 mg of the protein product with a first polypeptide that includes the amino acid sequence of SEQ ID NO: 3, and the second polypeptide that includes the amino acid sequence of SEQ ID NO: 1 is administered to a subject once every three weeks.

### Pharmacokinetic (PK) Analysis Sampling in Humans

Serum samples for pharmacokinetic (PK) data analysis were collected before the start of the first dose and at the following time points after the first dose: on Day 1 immediately after the infusion and 4 hours after the start of the infusion; on Day 2 at least 24 hours after the Day 1 end of infusion; and on Days 8 and 15. At selected subsequent dosing occasions pre-dose, end-of-infusion and 2 to 8 hours after the end of infusion samples were collected on days 15, 29, 43. For later time points on days 57, 71 and 85, pre-dose samples were or were to be collected followed by once every 6 weeks PK sampling until 12 weeks, then once every 12 weeks PK sampling. In the expansion phase sparse PK sampling was conducted.

### Establishing Body Weight-Independent Dosing Regimen

Informed by the clinical and pre-clinical data, a new, body weight-independent dosing regimen for the administration of anti-PD-L1/TGFβ Trap molecules has been created to achieve less variability in exposure, reduce dosing errors, reduce the time necessary for dose preparation, and reduce drug wastage compared to the mg/kg dosing, thus facilitating favorable treatment outcomes. According to one embodiment, a flat dose of at least 500 mg can be administered, regardless of the patient's body weight. According to another embodiments, a flat dose of at least 1200 mg can be administered, regardless of the patient's body weight. Typically, such a dose would be administered repeatedly, such as once every two weeks or once every 3 weeks, for example.

The PK data from the “PK Analysis Sampling in Humans” described above were used to produce a population PK model and to perform simulations of possible dosing regimens. Modeling method, known as the full approach model, described in Gastonguay, M., Full Covariate Models as an Alternative to Methods Relying on Statistical Significance for Inferences about Covariate Effects: A Review of Methodology and 42 Case Studies, (2011) p. 20, Abstract 2229, was applied to the population model data obtained from the simulations to obtain parameters having the following features: 2-compartment PK model with linear elimination, IIV on CL, V1, and V2, combined additive and proportional residual error, full covariate model on CL and V1. The following baseline covariates were included in the final model: age, weight, sex, race, albumin, CRP, platelet count, eGFR, hepatic impairment, ECOG score, tumor size, tumor type, and previous treatment with biologics. The following estimates of typical parameter estimates of pharmacokinetics of the protein of the present disclosure (e.g., anti-PD-L1/TGFβ Trap) were obtained: clearance (CL) 0.0177 L/h (6.2%), central volume of distribution (V1) 3.64 L (8.81%), peripheral volume of distribution (V2) 0.513 L (25.1%), and intercompartmental clearance (Q) 0.00219 L/h (17.8%). The inter-patient variability was 22% for CL, 20% for V1, and 135% for V2. Body weight was a relevant covariate on both CL and V1. To support the flat dosing approach, the impact of the dosing strategy on the exposure variability of the protein of the present disclosure (e.g., anti-PD-L1/TGFβ Trap) was explored. Specifically, simulations were performed to compare the exposure distribution using a flat dosing approach of 1200 mg once every two weeks versus a BW-adjusted dosing approach of either 17.65 mg/kg once every two weeks (corresponding to 1200 mg once every two weeks for a 68 kg subject or 15 mg/kg once every two weeks (corresponding to 1200 mg for a 80 kg subject). Further simulations were performed to compare the exposure distribution using a flat dosing approach of 500 mg once every two weeks versus a BW-adjusted dosing approach of 7.35 mg/kg once every two weeks (corresponding to 500 mg once every two weeks for a 68 kg subject). In addition, simulations were performed to asses the following flat doses at once every three weeks: 1200 mg, 1400, mg, 1600 mg, 1800 mg, 2000 mg, 2200 mg, 2400 mg, 2600 mg, 2800 mg, 3000 mg.

The following methodology for simulations was used: N=200 sets of parameter estimates were drawn from multivariate normal distribution of parameter estimates, using the final PK model variance-covariance matrix. For each parameter estimate, 200 IIV estimates were drawn from OMEGA multivariate normal distribution, resulting in total 40000 (200×200) subjects. The original dataset (N=380) was resampled with replacement to generate 40000 sets of matched covariates and steady-state exposure metrics (AUC, C_{avg}, C_{trough} and Cₘₐₓ) were generated for each dosing regimen.

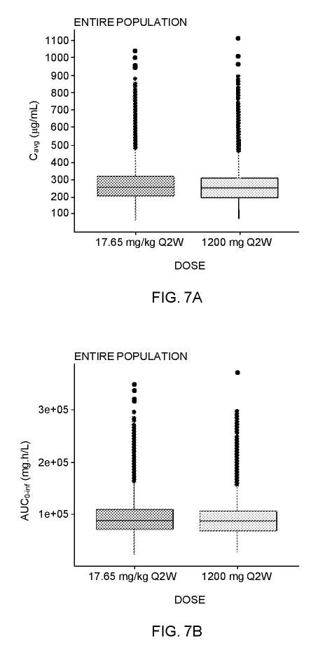

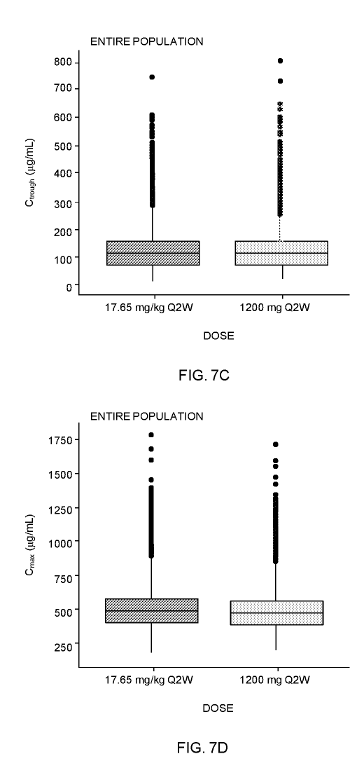

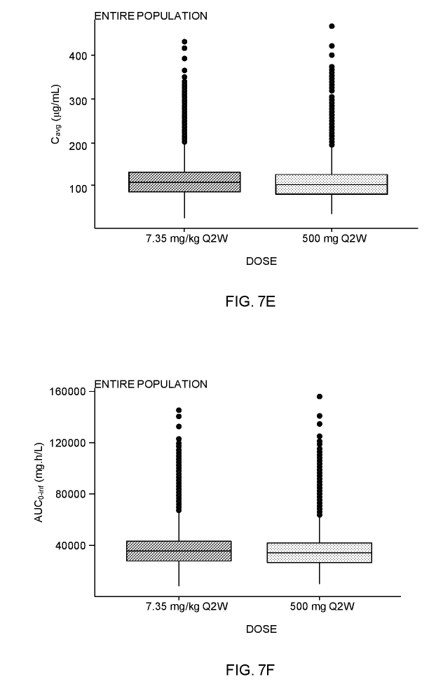

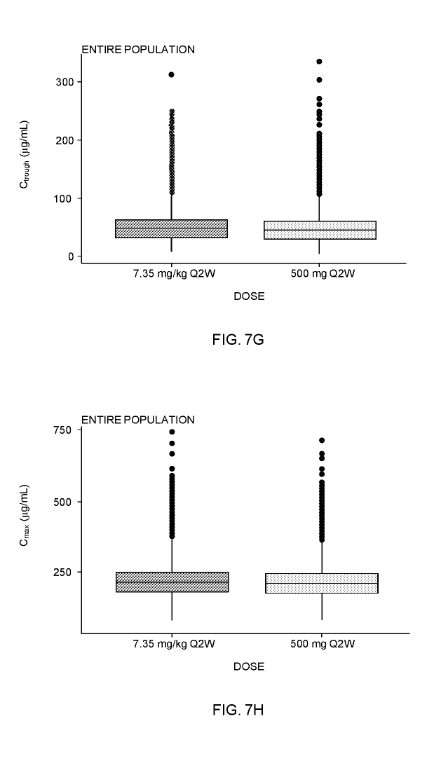

Simulations showed that across a wide BW spectrum, variability in exposure is slightly higher for BW-based dosing in comparison with fixed dosing. An example of exposure distribution at 17.65 mg/kg and 1200 mg flat dose, or 7.35 mg/kg and 500 mg flat dose for a median body weight of 68 kg is shown in FIGS. 7A and 7B, respectively. Simulations also showed the opposite trend in exposure distributions across weight quartiles across the patient population: low-weight patients have higher exposure with fixed dosing, whereas high-weight patients have higher exposure with BW-adjusted dosing.

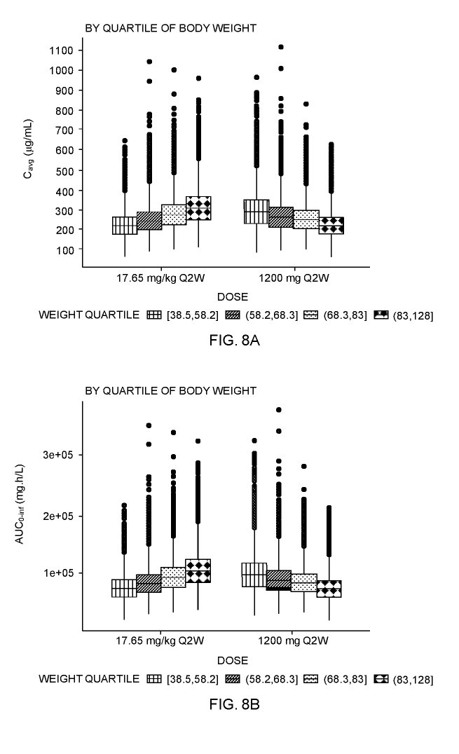

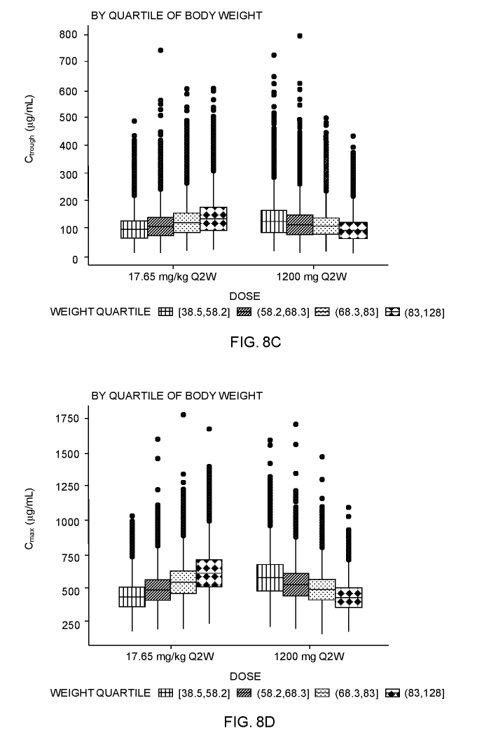

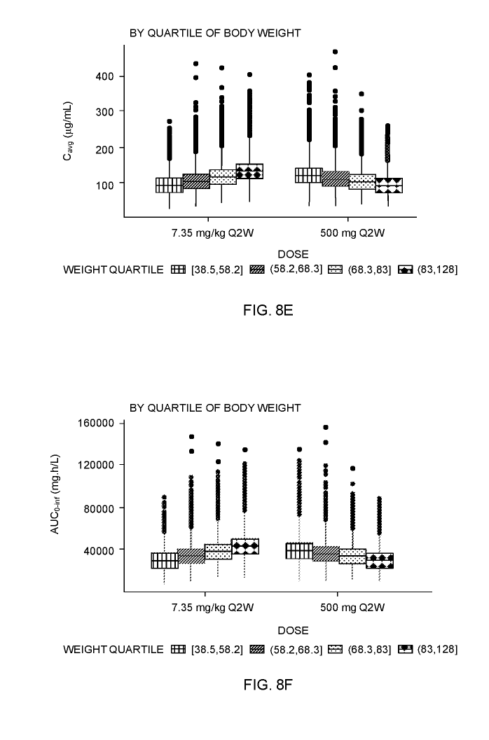

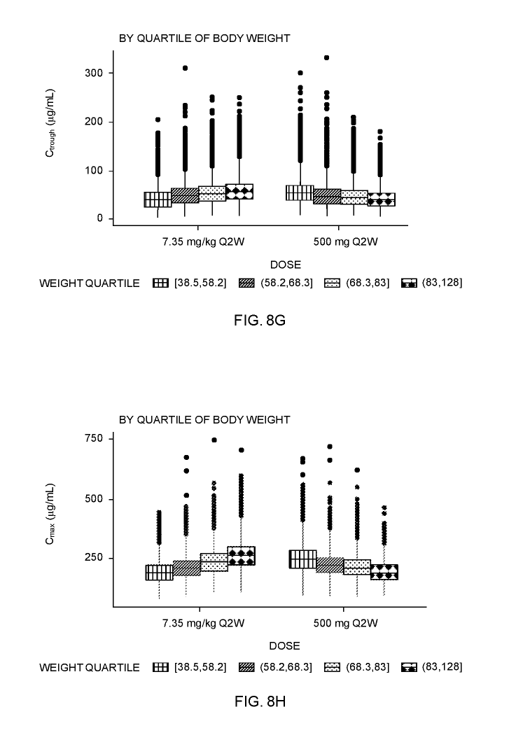

An example of exposure distribution across weight quartiles at 17.65 mg/kg and 1200 mg flat dose or 7.35 mg/kg and 500 mg flat dose for a median body weight of 68 kg is shown in FIGS. 8A and 8B, respectively.

Establishing Efficacious Dose/Dosing Regimen and Exposure in Humans: Preliminary Dose-Response and Exposure-Response in 2^{nd} Line Non Small Cell Lung Cancer (2L NSCLC) Following Once Every 2 Weeks (q2w) Dosing of Anti-PD-L1/TGFβ Trap

In one aspect, dose-response and exposure-response assessments are based on data from 80 subjects that were administered either 500 mg or 1200 mg of anti-PD-L1/TGFβ Trap once every 2 weeks (q2w) (n=40 per cohort) in the treatment of 2L NSCLC. Dose-response and exposure-response of the subjects were assessed. As of data cut-off at the time of analysis, a total of 17 subjects remained on treatment with a median follow-up of 35.2 (range, 1.3-47.3) weeks. The investigator-assessed unconfirmed overall response rate (ORR) was 25.0% (500 mg ORR, 22.5%; 1200 mg ORR, 27.5%), with 9 partial responses (PR) seen at 500 mg, and 1 complete response (CR) and 10 PRs at 1200 mg. Clinical activity was observed across PD-L1 expression levels with ORR of 71.4% at 1200 mg noted in patients with ≥80% PD-L1 tumor expression (7 patient). The most common treatment-related adverse events (TRAEs) were pruritus (18.8%), maculopapular rash (17.5%), decreased appetite (12.5%), and asthenia (11.3%). Grade ≥3 TRAEs occurred in 20 patients (25%). No treatment-related deaths occurred.

For the exposure-response assessment, the population PK model described above was used to predict first-cycle exposures based on dosing and covariate information from these 80 patients. Specifically, AUC and C_{trough} after a single dose were predicted for every subject using empirical Bayes estimates of population PK parameters (Table 2 and 3).

TABLE 2
Summary of predicted AUC_{0-336h,sd} by cohort

| Cohort | Median (mg * h/L) | 2.5-97.5 Percentile(mg * h/L) |
|---|---|---|
| 500 mg q2w | 27346 | 14561-59193 |
| 1200 mg q2w | 64981 | 47449-84799 |

AUC_{0-336h, sd} = AUC over 0-336 hr. period following a single dose, as predicted using a population PK model.

TABLE 3
Summary of predicted C_{trough,sd} by cohort

| Cohort | Median (μg/mL) | 2.5-97.5 Percentile (μg/mL) |
|---|---|---|
| 500 mg q2w | 31.82 | 9.803-100.6 |
| 1200 mg q2w | 78.76 | 51.25-136.2 |

C_{trough, sd} = C_{trough} following a single dose, as predicted using a population PK model.

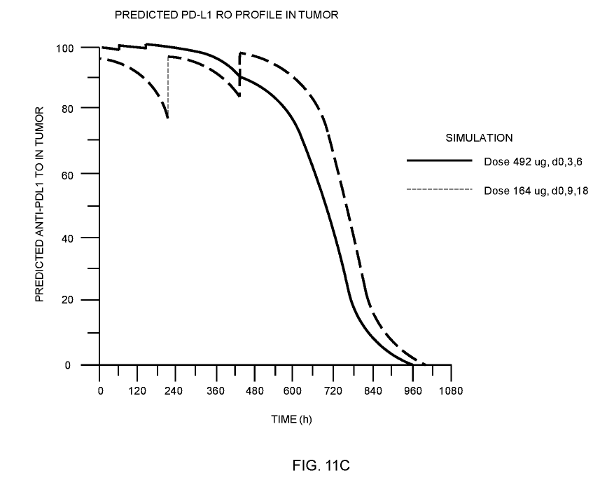

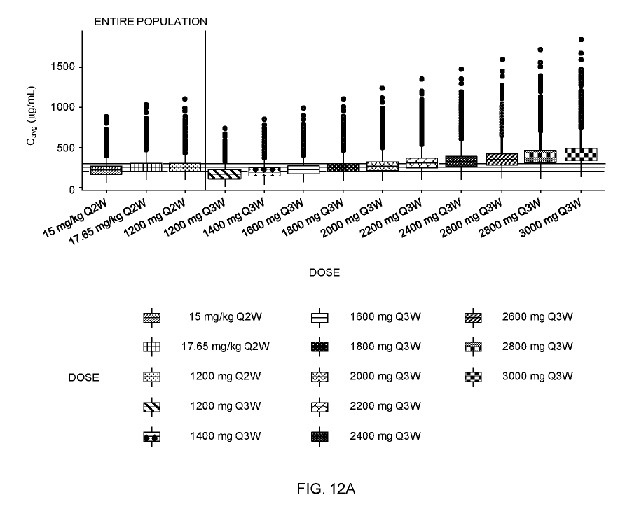

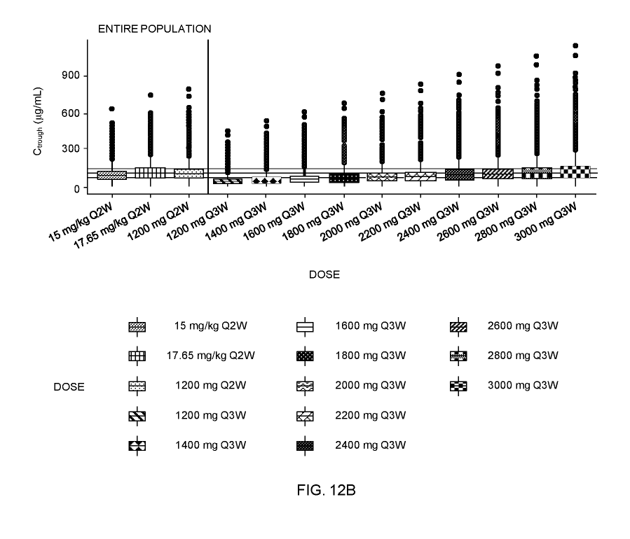

Predicted exposure data were combined for 500 mg q2w and 1200 mg q2w cohorts to calculate a response rate for each quartile of predicted exposure, as shown in Table 4 and Table 5 below. These preliminary data suggest that 1200 mg q2w may provide a more favorable efficacy profile compared to 500 mg q2w. Furthermore, these data suggest that the range of exposures achieved with 1200 mg q2w dosing regimen are associated with response (per RECIST v1.1) in 2L NSCLC and that this exposure range can be used to design alternative dosing regimens as shown in the example below (FIG. 12).

TABLE 4
Observed response rate by AUC_{0-336h,sd} in 2L NSCLC subjects
treated with either 500 mg or 1200 mg of anti-PD-L1/TGFβ Trap
once every 2 weeks

| | Number of | | | |
|---|---|---|---|---|
| | responders | Number of | Total Number of | Total |
| AUC_{0-336h, sd} | in 500 mg | responders in | responders per | Response |
| Quartile | cohort | 1200 mg cohort | quartile | rate |
| 0-25% | 2/20 | 0/0 | 2/20 | 10% |
| 25-50% | 5/18 | 0/2 | 5/20 | 25% |
| 50-75% | 1/1 | 4/19 | 5/20 | 25% |
| 75-100% | 0/1 | 6/19 | 6/20 | 30% |

AUC_{0-336h, sd} = AUC over 0-336 hr. period following a single dose, as predicted using a population PK model.

TABLE 5
Observed response rate by C_{trough,sd} in 2L NSCLC subjects
treated with either 500 mg or 1200 mg of anti-PD-L1/TGFβ Trap
once every 2 weeks (n = 40 per dose group).

| | Number of | Number of | | Total |
|---|---|---|---|---|
| C_{trough, sd} | responders in | responders in | Total Number | Response |
| Quartile | 500 mg cohort | 1200 mg cohort | of responders | rate |
| 0-25% | 1/20 | 0/0 | 1/20 | 5% |
| 25-50% | 5/15 | 0/5 | 5/20 | 25% |
| 50-75% | 1/3 | 3/17 | 4/20 | 20% |
| 75-100% | 1/2 | 7/18 | 8/20 | 40% |

C_{trough, sd} = C_{trough} following a single dose, as predicted using a population PK model.

Establishing Dosing Regimen with Various Dosing Frequencies

Data regimens with various dosing frequencies have been created to allow less frequent administration and/or to allow coordination of dosing schedules with concomicant medications. Specifically, the preliminary population PK modeling and simulation methodology described above has been used to simulate exposures for various dosing regimens and to compare regimens based on exposure.

Based on these simulations, a flat dose of at least 500 mg administered once every two weeks is required to maintain an average concentration of about 100 μg/mL for a typical subject, while a flat dose of about 1200 mg administered once every two weeks is required to maintain a C_{trough} of about 100 μg/mL.

Based on simulations for C_{avg}, 1200 mg once every two weeks is equivalent to 1800 mg once every three weeks (FIG. 12A), while for C_{trough}, 1200 mg once every two weeks is equivalent to 2800 mg once every three weeks (FIG. 12B). And for C_{avg}, 500 mg once every two weeks is equivalent to 750 mg once every three weeks; for C_{trough} 500 mg once every two weeks is equivalent to 1,167 mg once every three weeks.

### TGFβ as a Cancer Target

The current disclosure permits localized reduction in TGFβ in a tumor microenvironment by capturing the TGFβ using a soluble cytokine receptor (TGFβRII) tethered to an antibody moiety targeting a cellular immune checkpoint receptor found on the exterior surface of certain tumor cells or immune cells. An example of an antibody moiety of the disclosure to an immune checkpoint protein is anti-PD-L1. This bifunctional molecule, sometimes referred to in this document as an “antibody-cytokine Trap,” is effective precisely because the anti-receptor antibody and cytokine Trap are physically linked. The resulting advantage (over, for example, administration of the antibody and the receptor as separate molecules) is partly because cytokines function predominantly in the local environment through autocrine and paracrine functions. The antibody moiety directs the cytokine Trap to the tumor microenvironment where it can be most effective, by neutralizing the local immunosuppressive autocrine or paracrine effects. Furthermore, in cases where the target of the antibody is internalized upon antibody binding, an effective mechanism for clearance of the cytokine/cytokine receptor complex is provided. Antibody-mediated target internalization was shown for PD-L1, and anti-PD-L1/TGFβ Trap was shown to have a similar internalization rate as anti-PD-L1. This is a distinct advantage over using an anti-TGFβ antibody because first, an anti-TGFβ antibody might not be completely neutralizing; and second, the antibody can act as a carrier extending the half-life of the cytokine.

Indeed, as described below, treatment with the anti-PD-L1/TGFβ Trap elicits a synergistic anti-tumor effect due to the simultaneous blockade of the interaction between PD-L1 on tumor cells and PD-1 on immune cells, and the neutralization of TGFβ in the tumor microenvironment. Without being bound by theory, this presumably is due to a synergistic effect obtained from simultaneous blocking the two major immune escape mechanisms, and in addition, the depletion of the TGFβ in the tumor microenvironment by a single molecular entity. This depletion is achieved by (1) anti-PD-L1 targeting of tumor cells; (2) binding of the TGFβ autocrine/paracrine in the tumor microenvironment by the TGFβ Trap; and (3) destruction of the bound TGFβ through the PD-L1 receptor-mediated endocytosis. Furthermore, the TGFβRII fused to the C-terminus of Fc (fragment of crystallization of IgG) was several-fold more potent than the TGFβRII-Fc that places the TGFβRII at the N-terminus of Fc.

TGFβ had been a somewhat questionable target in cancer immunotherapy because of its paradoxical roles as the molecular Jekyll and Hyde of cancer (Bierie et al., Nat. Rev. Cancer, 2006; 6:506-20). Like some other cytokines, TGFβ activity is developmental stage and context dependent. Indeed TGFβ can act as either a tumor promoter or a tumor suppressor, affecting tumor initiation, progression and metastasis. The mechanisms underlying this dual role of TGFβ remain unclear (Yang et al., Trends Immunol. 2010; 31:220-227). Although it has been postulated that Smad-dependent signaling mediates the growth inhibition of TGFβ signaling, while the Smad independent pathways contribute to its tumor-promoting effect, there are also data showing that the Smad-dependent pathways are involved in tumor progression (Yang et al., Cancer Res. 2008; 68:9107-11).

Both the TGFβ ligand and the receptor have been studied intensively as therapeutic targets. There are three ligand isoforms, TGFβ1, 2 and 3, all of which exist as homodimers. There are also three TGFβ receptors (TGFβR), which are called TGFβR type I, II and III (López-Casillas et al., J Cell Biol. 1994; 124:557-68). TGFβRI is the signaling chain and cannot bind ligand. TGFβRII binds the ligand TGFβ1 and 3, but not TGFβ2, with high affinity. The TGFβRII/TGFβ complex recruits TGFβRI to form the signaling complex (Won et al., Cancer Res. 1999; 59:1273-7). TGFβRIII is a positive regulator of TGFβ binding to its signaling receptors and binds all 3 TGFβ isoforms with high affinity. On the cell surface, the TGFβ/TGFβRIII complex binds TGFβRII and then recruits TGFβRI, which displaces TGFβRIII to form the signaling complex.

Although the three different TGFβ isoforms all signal through the same receptor, they are known to have differential expression patterns and non-overlapping functions in vivo. The three different TGF-β isoform knockout mice have distinct phenotypes, indicating numerous non-compensated functions (Bujak et al., Cardiovasc Res. 2007; 74:184-95). While TGFβ1 null mice have hematopoiesis and vasculogenesis defects and TGFβ3 null mice display pulmonary development and defective palatogenesis, TGFβ2 null mice show various developmental abnormalities, the most prominent being multiple cardiac deformities (Bartram et al., Circulation. 2001; 103:2745-52; Yamagishi et al., Anat Rec. 2012; 295:257-67). Furthermore, TGFβ is implicated to play a major role in the repair of myocardial damage after ischemia and reperfusion injury. In an adult heart, cardiomyocytes secrete TGFβ, which acts as an autocrine to maintain the spontaneous beating rate. Importantly, 70-85% of the TGFβ secreted by cardiomyocytes is TGFβ2 (Roberts et al., J Clin Invest. 1992; 90:2056-62). Despite cardiotoxicity concerns raised by treatment with TGFβRI kinase inhibitors, present applicants have observed a lack of toxicity, including cardiotoxicity, for anti-PD-L1/TGFβ Trap in monkeys.

Therapeutic approaches to neutralize TGFβ include using the extracellular domains of TGFβ receptors as soluble receptor traps and neutralizing antibodies. Of the receptor Trap approach, soluble TGFβRIII may seem the obvious choice since it binds all the three TGFβ ligands. However, TGFβRIII, which occurs naturally as a 280-330 kD glucosaminoglycan (GAG)-glycoprotein, with extracellular domain of 762 amino acid residues, is a very complex protein for biotherapeutic development. The soluble TGFβRIII devoid of GAG could be produced in insect cells and shown to be a potent TGFβ neutralizing agent (Vilchis-Landeros et al, Biochem J 355:215, 2001). The two separate binding domains (the endoglin-related and the uromodulin-related) of TGFβRIII could be independently expressed, but they were shown to have affinities 20 to 100 times lower than that of the soluble TGFβRIII, and much diminished neutralizing activity (Mendoza et al., Biochemistry. 2009; 48:11755-65). On the other hand, the extracellular domain of TGFβRII is only 136 amino acid residues in length and can be produced as a glycosylated protein of 25-35 kD. The recombinant soluble TGFβRII was further shown to bind TGFβ1 with a K_{D} of 200 pM, which is fairly similar to the K_{D} of 50 pM for the full length TGFβRII on cells (Lin et al., J Biol Chem. 1995; 270:2747-54). Soluble TGFβRII-Fc was tested as an anti-cancer agent and was shown to inhibit established murine malignant mesothelioma growth in a tumor model (Suzuki et al., Clin. Cancer Res., 2004; 10:5907-18). Because TGFβRII does not bind TGFβ2, and TGFβRIII binds TGFβ1 and 3 with lower affinity than TGFβRII, a fusion protein of the endoglin domain of TGFβRIII and extracellular domain of TGFβRII was produced in bacteria and was shown to inhibit the signaling of TGFβ1 and 2 in cell based assays more effectively than either TGFβRII or RIII (Verona et al., Protein Eng Des Sel. 2008; 21:463-73).

Still another approach to neutralize all three isoforms of the TGFβ ligands is to screen for a pan-neutralizing anti-TGFβ antibody, or an anti-receptor antibody that blocks the receptor from binding to TGFβ1, 2 and 3. GC1008, a human antibody specific for all isoforms of TGFβ, was in a Phase I/II study in patients with advanced malignant melanoma or renal cell carcinoma (Morris et al., J Clin Oncol 2008; 26:9028 (Meeting abstract)). Although the treatment was found to be safe and well tolerated, only limited clinical efficacy was observed, and hence it was difficult to interpret the importance of anti-TGFβ therapy without further characterization of the immunological effects (Flavell et al., Nat Rev Immunol. 2010; 10:554-67). There were also TGFβ-isoform-specific antibodies tested in the clinic. Metelimumab, an antibody specific for TGFβ1 was tested in Phase 2 clinical trial as a treatment to prevent excessive post-operative scarring for glaucoma surgery; and Lerdelimumab, an antibody specific for TGFβ2, was found to be safe but ineffective at improving scarring after eye surgery in a Phase 3 study (Khaw et al., Ophthalmology 2007; 114:1822-1830). Anti-TGFβRII antibodies that block the receptor from binding to all the three TGFβ isoforms, such as the anti-human TGFβRII antibody TR1 and anti-mouse TGFβRII antibody MT1, have also shown some therapeutic efficacy against primary tumor growth and metastasis in mouse models (Zhong et al., Clin Cancer Res. 2010; 16:1191-205). However, in a recent Phase I study of antibody TR1 (LY3022859), dose escalation beyond 25 mg (flat dose) was considered unsafe due to uncontrolled cytokine release, despite prophylactic treatment (Tolcher et al., Cancer Chemother Pharmacol 2017; 79:673-680). To date, the vast majority of the studies on TGFβ targeted anticancer treatment, including small molecule inhibitors of TGFβ signaling that often are quite toxic, are mostly in the preclinical stage and the anti-tumor efficacy obtained has been limited (Calone et al., Exp Oncol. 2012; 34:9-16; Connolly et al., Int J Biol Sci. 2012; 8:964-78).

The antibody-TGFβ Trap of the disclosure is a bifunctional protein containing at least portion of a human TGFβ Receptor II (TGFβRII) that is capable of binding TGFβ. In certain embodiments, the TGFβ Trap polypeptide is a soluble portion of the human TGFβ Receptor Type 2 Isoform A (SEQ ID NO: 8) that is capable of binding TGFβ. In certain embodiments, TGFβ Trap polypeptide contains at least amino acids 73-184 of SEQ ID NO: 8. In certain embodiments, the TGFβ Trap polypeptide contains amino acids 24-184 of SEQ ID NO: 8. In certain embodiments, the TGFβ Trap polypeptide is a soluble portion of the human TGFβ Receptor Type 2 Isoform B (SEQ ID NO: 9) that is capable of binding TGFβ. In certain embodiments, TGFβ Trap polypeptide contains at least amino acids 48-159 of SEQ ID NO: 9. In certain embodiments, the TGFβ Trap polypeptide contains amino acids 24-159 of SEQ ID NO: 9. In certain embodiments, the TGFβ Trap polypeptide contains amino acids 24-105 of SEQ ID NO: 9.

### Mechanisms of Action

The approach of targeting T cell inhibition checkpoints for dis-inhibition with therapeutic antibodies is an area of intense investigation (for a review, see Pardoll, Nat Rev Cancer. 2012; 12:253-264). In one approach, the antibody moiety or antigen binding fragment thereof targets T cell inhibition checkpoint receptor proteins on the T cell, such as, for example: CTLA-4, PD-1, BTLA, LAG-3, TIM-3, or LAIR1. In another approach, the antibody moiety targets the counter-receptors on antigen presenting cells and tumor cells (which co-opt some of these counter-receptors for their own immune evasion), such as for example: PD-L1 (B7-H1), B7-DC, HVEM, TIM-4, B7-H3, or B7-H4.

The disclosure contemplates antibody TGFβ traps that target, through their antibody moiety or antigen binding fragment thereof, T cell inhibition checkpoints for dis-inhibition. To that end the applicants have tested the anti-tumor efficacy of combining a TGFβ Trap with antibodies targeting various T cell inhibition checkpoint receptor proteins, such as anti-PD-1, anti-PD-L1, anti-TIM-3 and anti-LAGS.

The programmed death 1 (PD-1)/PD-L1 axis is an important mechanism for tumor immune evasion. Effector T cells chronically sensing antigen take on an exhausted phenotype marked by PD-1 expression, a state under which tumor cells engage by upregulating PD-L1. Additionally, in the tumor microenvironment, myeloid cells, macrophages, parenchymal cells and T cells upregulate PD-L1. Blocking the axis restores the effector function in these T cells. Anti-PD-L1/TGFβ Trap also binds TGFβ (1, 2, and 3 isoforms), which is an inhibitory cytokine produced in the tumor microenvironment by cells including apoptotic neutrophils, myeloid-derived suppressor cells, T cells and tumor. Inhibition of TGFβ by soluble TGFβRII reduced malignant mesothelioma in a manner that was associated with increases in CD8+ T cell anti-tumor effects. The absence of TGFβ1 produced by activated CD4+ T cells and Treg cells has been shown to inhibit tumor growth, and protect mice from spontaneous cancer. Thus, TGFβ appears to be important for tumor immune evasion.

TGFβ has growth inhibitory effects on normal epithelial cells, functioning as a regulator of epithelial cell homeostasis, and it acts as a tumor suppressor during early carcinogenesis. As tumors progress toward malignancy, the growth inhibitory effects of TGFβ on the tumor are lost via mutation in one or more TGFβ pathway signaling components or through oncogenic reprogramming Upon loss of sensitivity to TGFβ inhibition, the tumor continues to produce high levels of TGFβ, which then serve to promote tumor growth. The TGFβ cytokine is overexpressed in various cancer types with correlation to tumor stage. Many types of cells in the tumor microenvironment produce TGFβ including the tumor cells themselves, immature myeloid cells, regulatory T cells, and stromal fibroblasts; these cells collectively generate a large reservoir of TGFβ in the extracellular matrix. TGFβ signaling contributes to tumor progression by promoting metastasis, stimulating angiogenesis, and suppressing innate and adaptive anti-tumor immunity. As a broadly immunosuppressive factor, TGFβ directly down-regulates the effector function of activated cytotoxic T cells and NK cells and potently induces the differentiation of naïve CD4+ T cells to the immunosuppressive regulatory T cells (Treg) phenotype. In addition, TGFβ polarizes macrophages and neutrophils to a wound-healing phenotype that is associated with production of immunosuppressive cytokines. As a therapeutic strategy, neutralization of TGFβ activity has the potential to control tumor growth by restoring effective anti-tumor immunity, blocking metastasis, and inhibiting angiogenesis.

Combining these pathways, PD-1 or PD-L1, and TGFβ, is attractive as an antitumor approach. Concomitant PD-1 and TGFβ blockade can restore pro-inflammatory cytokines. Anti-PD-L1/TGFβ Trap includes, for example, an extracellular domain of the human TGFβ receptor TGFβRII covalently joined via a glycine/serine linker to the C terminus of each heavy chain of the fully human IgG1 anti-PD-L1 antibody. Given the emerging picture for PD-1/PD-L1 class, in which responses are apparent but with room for increase in effect size, it is assumed that co-targeting a complementary immune modulation step will improve tumor response. A similar TGF-targeting agent, fresolimumab, which is a monoclonal antibody targeting TGFβ1, 2 and 3, showed initial evidence of tumor response in a Phase I trial in subjects with melanoma.

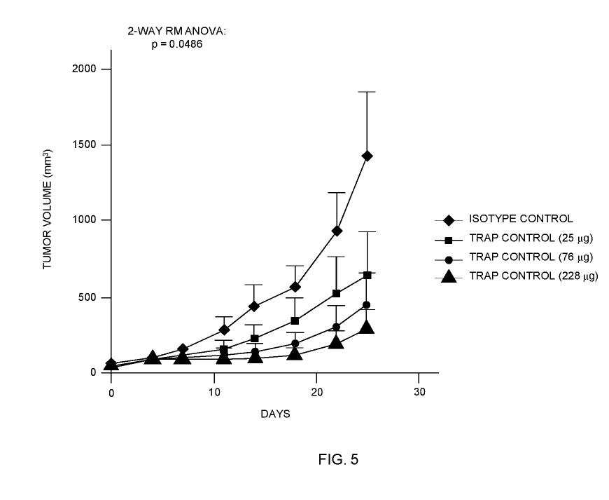

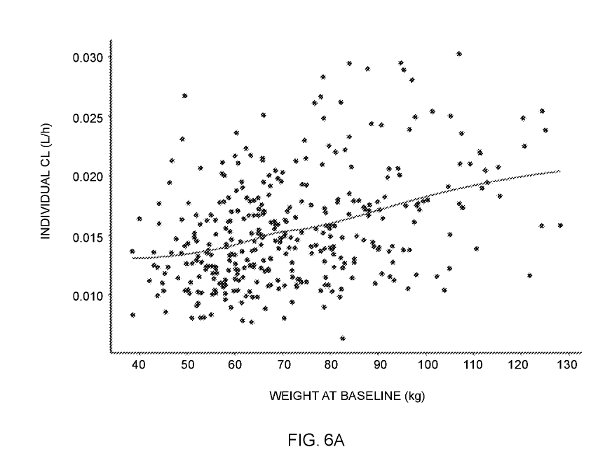

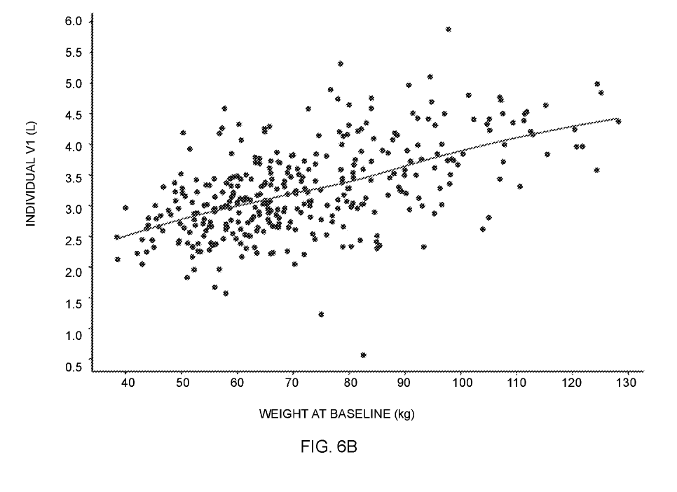

In certain embodiments, the present disclosure provides experiments, which demonstrated that the TGFβRII portion of anti-PD-L1/TGFβ Trap (the Trap control “anti-PDL-1(mut)/TGFβ Trap”) elicited antitumor activity. For example, following subcutaneous implantation in a Detroit 562 human pharyngeal carcinoma model, anti-PDL-1(mut)/TGFβ Trap elicited a dose-dependent reduction in tumor volume when administered at 2.5 μg, 76 μg, or 228 μg (FIG. 5).

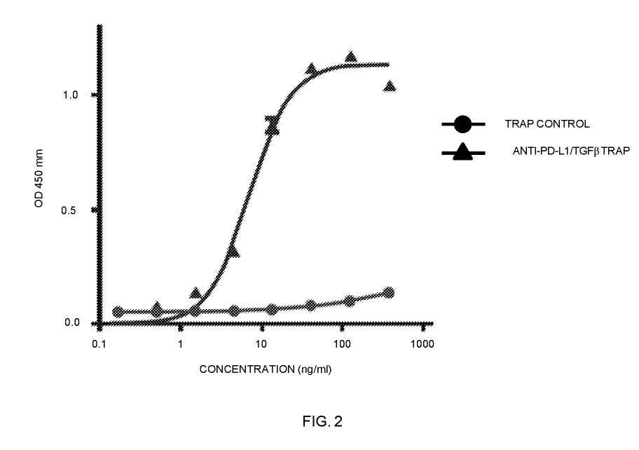

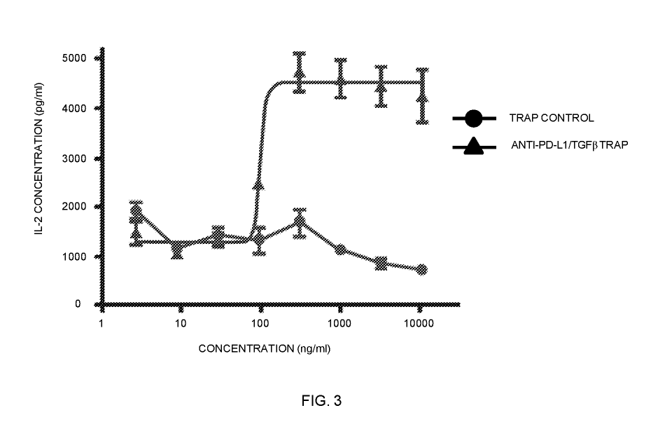

In certain embodiments, the present disclosure provides experiments, which demonstrated that the protein of the present disclosure simultaneously bound to both PD-L1 and TGFβ (FIG. 2).

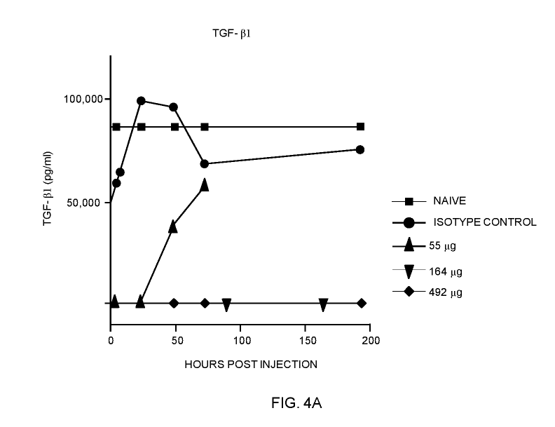

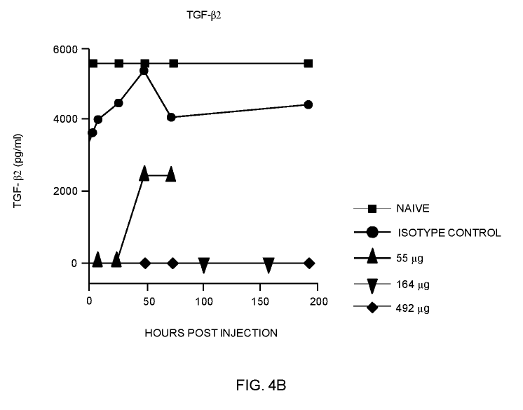

In certain embodiments, the present disclosure provides experiments, which demonstrated that the protein of the present disclosure (e.g. anti-PD-L1/TGFβ Trap) inhibited PD-L1 and TGFβ dependent signaling in vitro. In certain embodiments, the present disclosure provides experiments, which demonstrated that the protein of the present disclosure enhanced T cell effector function in vitro via blockade of PD-L1-mediated immune inhibition as measured by an IL-2 induction assay following superantigen stimulation (FIG. 3). At approximately 100 ng/ml, the protein of the present disclosure induced a dramatic increase in IL-2 levels in vitro (FIG. 3).

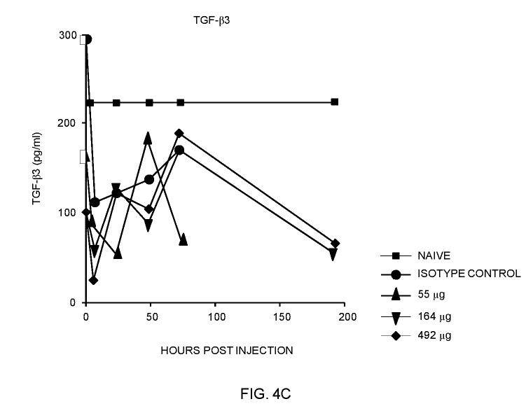

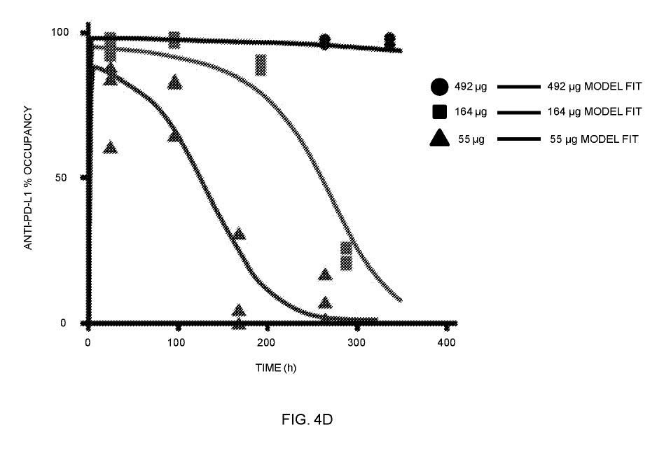

In certain embodiments, the present disclosure provides experiments, which demonstrated that the protein of the present disclosure (e.g. anti-PD-L1/TGFβ Trap) caused depletion of TGFβ from blood in vivo. Treatment of orthotopically implanted EMT-6 breast cancer cells in JH mice with 55 μg, or 164 μg, or 492 μg of the protein of the present disclosure resulted in efficient and specific depletion of TGFβ1 (FIG. 4A), TGFβ2 (FIG. 4B), and TGFβ3 (FIG. 4C). Furthermore, the present disclosure provides experiments, which demonstrated that the protein of the present disclosure occupied the PD-L1 target, supporting the notion that that the protein of the present disclosure fit to a receptor binding model in the EMT-6 tumor system (FIG. 4D).

In certain embodiments, the present disclosure provides experiments, which demonstrated that the protein of the present disclosure efficiently, specifically, and simultaneously bound to PD-L1 and TGFβ, possessed potent antitumor activity in a variety of mouse models, suppressed tumor growth and metastasis, as well as extended survival and conferred long-term protective antitumor immunity.

In a first-in-human phase I dose escalation study, in addition to monitoring the pharmacokinetics of the anti-PD-L1/TGFβ Trap molecule the mechanism of action, particularly against the TGFβ cytokines, was investigated.

Patients were treated with anti-PD-L1/TGFβ Trap molecule intravenously administered at 5 dose levels of about 0.3, about 1, about 3, about 10, or about 20 mg/kg once every two weeks, PK analyses were performed from samples for up to day 85. PD-L1 target occupancy was measured in CD3+ PBMCs by flow cytometry from patient blood collected at pre-dose, Day 2 (D2), D15, and D43. Further, the blood levels of TGFβ1-3 and pro-inflammatory cytokines were measured at these time points with an additional time point at D8 using analytically validated Luminex bead- and ECLIA-based multiplex immunoassays. In one aspect, patients can be treated with anti-PD-L1/TGFβ Trap molecule intravenously administered at 6 dose levels, including the ones described above, at a dose of about 30 mg/kg or about 40 mg/kg every two weeks. PK analyses of patients treated at 6 dose levels may be performed from samples for up to after the 6th dose. PD-L1 target occupancy may also be measured in CD3+ PBMCs by flow cytometry from patient blood collected at pre-dose, Day 2 (D2), D15, D43, and up to D85. Further, the blood levels of TGFβ1-3 and pro-inflammatory cytokines may be measured at these time points with an additional time point, e.g., at D8, using analytically validated Luminex bead- and ECLIA-based multiplex immunoassays.

Results indicated that the anti-PD-L1/TGFβ Trap molecule PK exposure during the first cycle increased in an approximately dose-proportional manner between 3 to 20 mg/kg, without significant accumulation within the first 85 days of treatment. There was about 80% PD-L1 target occupancy at 3 mg/kg-20 mg/kg which was maintained throughout the dosing interval. There was further a small (1.7 fold on D2) but significant induction of IFNγ at 0.3-20 mg/kg (p=0.001, n=19). Levels of TGFβ1, TGFβ2, and TGFβ3 in blood were reduced by a minimum of 99%, 92%, and 91%, respectively, at all-time points for dose levels 1-20 mg/kg. At the lower dose of 0.3 mg/kg, TGFβ1-3 levels were depleted at D2 and D8, but not at D15. Moreover, there was further a strong correlation between the drug PK levels and the TGFβ trapping. Thus, complete TGFβ1-3 trapping was achieved at a drug dose level of 1 mg/kg or above.

### Anti-PD-L1 Antibodies

The disclosure can include any anti-PD-L1 antibody, or antigen-binding fragment thereof, described in the art. Anti-PD-L1 antibodies are commercially available, for example, the 29E2A3 antibody (Biolegend, Cat. No. 329701). Antibodies can be monoclonal, chimeric, humanized, or human. Antibody fragments include Fab, F(ab′)2, scFv and Fv fragments, which are described in further detail below.

Exemplary antibodies are described in PCT Publication WO 2013/079174. These antibodies can include a heavy chain variable region polypeptide including an HVR-H1, HVR-H2, and HVR-H3 sequence, where:

(SEQ ID NO: 21)
(a) the HVR-H1 sequence is X₁YX₂MX₃;
(SEQ ID NO: 22)
(b) the HVR-H2 sequence is SIYPSGGX₄TFYADX₅VKG;
(SEQ ID NO: 23)
(c) the HVR-H3 sequence is IKLGTVTTVX₆Y;

further where: X₁ is K, R, T, Q, G, A, W, M, I, or S; X₂ is V, R, K, L, M, or I; X₃ is H, T, N, Q, A, V, Y, W, F, or M; X₄ is F or I; X₅ is S or T; X₆ is E or D.

In a one embodiment, X₁ is M, I, or S; X₂ is R, K, L, M, or I; X₃ is F or M; X₄ is F or I; X₅ is S or T; X₆ is E or D.

In another embodiment X₁ is M, I, or S; X₂ is L, M, or I; X₃ is F or M; X₄ is I; X₅ is S or T; X₆ is D.

In still another embodiment, X₁ is S; X₂ is I; X₃ is M; X₄ is I; X₅ is T; X₆ is D.

In another aspect, the polypeptide further includes variable region heavy chain framework sequences juxtaposed between the HVRs according to the formula: (HC-FR1)-(HVR-H1)-(HC-FR2)-(HVR-H2)-(HC-FR3)-(HVR-H3)-(HC-FR4).

In yet another aspect, the framework sequences are derived from human consensus framework sequences or human germline framework sequences.

In a still further aspect, at least one of the framework sequences is the following:

(SEQ ID NO: 24)
HC-FR1 is EVQLLESGGGLVQPGGSLRLSCAASGFTFS;
(SEQ ID NO: 25)
HC-FR2 is WVRQAPGKGLEWVS;
(SEQ ID NO: 26)
HC-FR3 is RFTISRDNSKNTLYLQMNSLRAEDTAVYYCAR;
(SEQ ID NO: 27)
HC-FR4 is WGQGTLVTVSS.

In a still further aspect, the heavy chain polypeptide is further combined with a variable region light chain including an HVR-L1, HVR-L2, and HVR-L3, where:

(SEQ ID NO: 28)
(a) the HVR-L1 sequence is TGTX₇X₈DVGX₉YNYVS;
(SEQ ID NO: 29)
(b) the HVR-L2 sequence is X₁₀VX₁₁X₁₂RPS;
(SEQ ID NO: 30)
(c) the HVR-L3 sequence is SSX₁₃TX₁₄X₁₅X₁₆X₁₇RV;

further where: X₇ is N or S; X₈ is T, R, or S; X₉ is A or G; X₁₀ is E or D; X₁₁ is I, N or S; X₁₂ is D, H or N; X₁₃ is F or Y; X₁₄ is N or S; X₁₅ is R, T or S; X₁₆ is G or S; X₁₇ is I or T.

In another embodiment, X₇ is N or S; X₈ is T, R, or S; X₉ is A or G; X₁₀ is E or D; X₁₁ is N or S; X₁₂ is N; X₁₃ is F or Y; X₁₄ is 5; X₁₅ is 5; X₁₆ is G or S; X₁₇ is T.

In still another embodiment, X₇ is S; X₈ is S; X₉ is G; X₁₀ is D; X₁₁ is S; X₁₂ is N; X₁₃ is Y; X₁₄ is S; X₁₅ is S; X₁₆ is S; X₁₇ is T.

In a still further aspect, the light chain further includes variable region light chain framework sequences juxtaposed between the HVRs according to the formula: (LC-FR1MHVR-L1)-(LC-FR2)-(HVR-L2)-(LC-FR3)-(HVR-L3)-(LC-FR4).

In a still further aspect, the light chain framework sequences are derived from human consensus framework sequences or human germline framework sequences.

In a still further aspect, the light chain framework sequences are lambda light chain sequences.

In a still further aspect, at least one of the framework sequence is the following:

(SEQ ID NO: 31)
LC-FR1 is QSALTQPASVSGSPGQSITISC;
(SEQ ID NO: 32)
LC-FR2 is WYQQHPGKAPKLMIY;
(SEQ ID NO: 33)
LC-FR3 is GVSNRFSGSKSGNTASLTISGLQAEDEADYYC;
(SEQ ID NO: 34)
LC-FR4 is FGTGTKVTVL.

In another embodiment, the disclosure provides an anti-PD-L1 antibody or antigen binding fragment including a heavy chain and a light chain variable region sequence, where:

(a) the heavy chain includes an HVR-H1, HVR-H2, and HVR-H3, wherein further: (i) the HVR-H1 sequence is X₁YX₂MX₃ (SEQ ID NO: 21); (ii) the HVR-H2 sequence is SIYPSGGX₄TFYADX₅VKG (SEQ ID NO: 22); (iii) the HVR-H3 sequence is IKLGTVTTVX₆Y (SEQ ID NO: 23), and;

(b) the light chain includes an HVR-L1, HVR-L2, and HVR-L3, wherein further: (iv) the HVR-L1 sequence is TGTX₇X₈DVGX₉YNYVS (SEQ ID NO: 28); (v) the HVR-L2 sequence is X₁₀VX₁₁X₁₂RPS (SEQ ID NO: 29); (vi) the HVR-L3 sequence is SSX₁₃TX₁₄X₁₅X₁₆X₁₇RV (SEQ ID NO: 30); wherein: X₁ is K, R, T, Q, G, A, W, M, I, or S; X₂ is V, R, K, L, M, or I; X₃ is H, T, N, Q, A, V, Y, W, F, or M; X₄ is F or I; X₅ is S or T; X₆ is E or D; X₇ is N or S; X₈ is T, R, or S; X₉ is A or G; X₁₀ is E or D; is I, N, or S; X₁₂ is D, H, or N; X₁₃ is F or Y; X₁₄ is N or S; X₁₅ is R, T, or S; X₁₆ is G or S; X₁₇ is I or T.

In one embodiment, X₁ is M, I, or S; X₂ is R, K, L, M, or I; X₃ is F or M; X₄ is F or I; X₅ is S or T; X₆ is E or D; X₇ is N or S; X₈ is T, R, or S; X₉ is A or G; X₁₀ is E or D; X₁₁ is N or S; X₁₂ is N; X₁₃ is F or Y; X₁₄ is S; X₁₅ is S; X₁₆ is G or S; X₁₇ is T.

In another embodiment, X₁ is M, I, or S; X₂ is L, M, or I; X₃ is F or M; X₄ is I; X₅ is S or T; X₆ is D; X₇ is N or S; X₈ is T, R, or S; X₉ is A or G; X₁₀ is E or D; is N or S; X₁₂ is N; X₁₃ is F or Y; X₁₄ is S; X₁₅ is S; X₁₆ is G or S; X₁₇ is T.

In still another embodiment, X₁ is S; X₂ is I; X₃ is M; X₄ is I; X₅ is T; X₆ is D; X₇ is S; X₈ is S; X₉ is G; X₁₀ is D; X₁₁ is S; X₁₂ is N; X₁₃ is Y; X₁₄ is S; X₁₅ is S; X₁₆ is S; X₁₇ is T.

In a further aspect, the heavy chain variable region includes one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions include one or more framework sequences juxtaposed between the HVRs as: (LC-FR1 MHVR-L1)-(LC-FR2)-(HVR-L2)-(LC-FR3)-(HVR-L3)-(LC-FR4).

In a still further aspect, the framework sequences are derived from human consensus framework sequences or human germline sequences.

In a still further aspect, one or more of the heavy chain framework sequences is the following:

(SEQ ID NO: 24)
HC-FR1 is EVQLLESGGGLVQPGGSLRLSCAASGFTFS;
(SEQ ID NO: 25)
HC-FR2 is WVRQAPGKGLEWVS;
(SEQ ID NO: 26)
HC-FR3 is RFTISRDNSKNTLYLQMNSLRAEDTAVYYCAR;
(SEQ ID NO: 27)
HC-FR4 is WGQGTLVTVSS.

In a still further aspect, the light chain framework sequences are lambda light chain sequences.

In a still further aspect, one or more of the light chain framework sequences is the following:

(SEQ ID NO: 31)
LC-FR1 is QSALTQPASVSGSPGQSITISC;
(SEQ ID NO: 32)
LC-FR2 is WYQQHPGKAPKLMIY;
(SEQ ID NO: 33)
LC-FR3 is GVSNRFSGSKSGNTASLTISGLQAEDEADYYC;
(SEQ ID NO: 34)
LC-FR4 is FGTGTKVTVL.

In a still further aspect, the heavy chain variable region polypeptide, antibody, or antibody fragment further includes at least a C_{H}1 domain.

In a more specific aspect, the heavy chain variable region polypeptide, antibody, or antibody fragment further includes a C_{H}1, a C_{H}2, and a C_{H}3 domain.

In a still further aspect, the variable region light chain, antibody, or antibody fragment further includes a C_{L} domain.

In a still further aspect, the antibody further includes a C_{H}1, a C_{H}2, a C_{H}3, and a C_{L} domain.

In a still further specific aspect, the antibody further includes a human or murine constant region.

In a still further aspect, the human constant region is selected from the group consisting of IgG1, IgG2, IgG2, IgG3, IgG4.

In a still further specific aspect, the human or murine constant region is IgG1.

In yet another embodiment, the disclosure features an anti-PD-L1 antibody including a heavy chain and a light chain variable region sequence, where:

(a) the heavy chain includes an HVR-H1, an HVR-H2, and an HVR-H3, having at least 80% overall sequence identity to SYIMM (SEQ ID NO: 35), SIYPSGGITFYADTVKG (SEQ ID NO: 36), and IKLGTVTTVDY (SEQ ID NO: 37), respectively, and

(b) the light chain includes an HVR-L1, an HVR-L2, and an HVR-L3, having at least 80% overall sequence identity to TGTSSDVGGYNYVS (SEQ ID NO: 38), DVSNRPS (SEQ ID NO: 39), and SSYTSSSTRV (SEQ ID NO: 40), respectively.

In a specific aspect, the sequence identity is 81%, 82%, 83%, 84%, 85%, 86%, 87%, 88%, 89%, 90%, 91%, 92%, 93%, 94%, 95%, 96%, 97%, 98%, 99%, or 100%.

In yet another embodiment, the disclosure features an anti-PD-L1 antibody including a heavy chain and a light chain variable region sequence, where:

(a) the heavy chain includes an HVR-H1, an HVR-H2, and an HVR-H3, having at least 80% overall sequence identity to MYMMM (SEQ ID NO: 41), SIYPSGGITFYADSVKG (SEQ ID NO: 42), and IKLGTVTTVDY (SEQ ID NO: 37), respectively, and

(b) the light chain includes an HVR-L1, an HVR-L2, and an HVR-L3, having at least 80% overall sequence identity to TGTSSDVGAYNYVS (SEQ ID NO: 43), DVSNRPS (SEQ ID NO: 39), and SSYTSSSTRV (SEQ ID NO: 40), respectively.

In a specific aspect, the sequence identity is 81%, 82%, 83%, 84%, 85%, 86%, 87%, 88%, 89%, 90%, 91%, 92%, 93%, 94%, 95%, 96%, 97%, 98%, 99%, or 100%.

In a still further aspect, in the antibody or antibody fragment according to the disclosure, as compared to the sequences of HVR-H1, HVR-H2, and HVR-H3, at least those amino acids remain unchanged that are highlighted by underlining as follows:

(a) in HVR-H1
(SEQ ID NO: 35)
SYIMM,
(b) in HVR-H2
(SEQ ID NO: 36)
SIYPSGGITFYADTVKG,
(c) in HVR-H3
(SEQ ID NO: 37)
IKLGTVTTVDY;

and further where, as compared to the sequences of HVR-L1, HVR-L2, and HVR-L3 at least those amino acids remain unchanged that are highlighted by underlining as follows:

(a) HVR-L1
(SEQ ID NO: 38)
TGTSSDVGGYNYVS
(b) HVR-L2
(SEQ ID NO: 39)
DVSNRPS
(c) HVR-L3
(SEQ ID NO: 40)
SSYTSSSTRV.

In another aspect, the heavy chain variable region includes one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions include one or more framework sequences juxtaposed between the HVRs as: (LC-FR1)-(HVR-L1)-(LC-FR2)-(HVR-L2)-(LC-FR3)-(HVR-L3)-(LC-FR4).

In yet another aspect, the framework sequences are derived from human germline sequences.

In a still further aspect, one or more of the heavy chain framework sequences is the following:

HC-FR1 is
(SEQ ID NO: 24)
EVQLLESGGGLVQPGGSLRLSCAASGFTFS;
HC-FR2 is
(SEQ ID NO: 25)
WVRQAPGKGLEWVS;
HC-FR3 is
(SEQ ID NO: 26)
RFTISRDNSKNTLYLQMNSLRAEDTAVYYCAR;
HC-FR4 is
(SEQ ID NO: 27)
WGQGTLVTVSS.

In a still further aspect, the light chain framework sequences are derived from a lambda light chain sequence.

In a still further aspect, one or more of the light chain framework sequences is the following:

LC-FR1 is
(SEQ ID NO: 31)
QSALTQPASVSGSPGQSITISC;
LC-FR2 is
(SEQ ID NO: 32)
WYQQHPGKAPKLMIY;
LC-FR3 is
(SEQ ID NO: 33)
GVSNRFSGSKSGNTASLTISGLQAEDEADYYC;
LC-FR4 is
(SEQ ID NO: 34)
FGTGTKVTVL.

In a still further specific aspect, the antibody further includes a human or murine constant region.

In a still further aspect, the human constant region is selected from the group consisting of IgG1, IgG2, IgG2, IgG3, IgG4.

In certain embodiments, the disclosure features an anti-PD-L1 antibody including a heavy chain and a light chain variable region sequence, where:

(a) the heavy chain sequence has at least 85% sequence identity to the heavy chain sequence:

(SEQ ID NO: 44)
EVQLLESGGGLVQPGGSLRLSCAASGFTFSSYIMMVWRQAPGKGLEWVSS
IYPSGGITFYADWKGRFTISRDNSKNTLYLQMNSLRAEDTAVYYCARIKL
GTVTTVDYWGQGTLVTVSS,

and

(b) the light chain sequence has at least 85% sequence identity to the light chain sequence:

(SEQ ID NO: 45)
QSALTQPASVSGSPGQSITISCTGTSSDVGGYNYVSWYQQHPGKAPKLMI
YDVSNRPSGVSNRFSGSKSGNTASLTISGLQAEDEADYYCSSYTSSSTRV
FGTGTKVTVL.

In a specific aspect, the sequence identity is 86%, 87%, 88%, 89%, 90%, 91%, 92%, 93%, 94%, 95%, 96%, 97%, 98%, 99%, or 100%.

In certain embodiments, the disclosure provides for an anti-PD-L1 antibody including a heavy chain and a light chain variable region sequence, where:

(a) the heavy chain sequence has at least 85% sequence identity to the heavy chain sequence:

(SEQ ID NO: 46)
EVQLLESGGGLVQPGGSLRLSCAASGFTFSMYMMMWVRQAPGKGLEVWSS
IYPSGGITFYADSVKGRFTISRDNSKNTLYLQMNSLRAEDTAIYYCARIK
LGTVTTVDYWGQGTLVTVSS,

and

(b) the light chain sequence has at least 85% sequence identity to the light chain sequence:

(SEQ ID NO: 47)
QSALTQPASVSGSPGQSITISCTGTSSDVGAYNYVSWYQQHPGKAPKLMI
YDVSNRPSGVSNRFSGSKSGNTASLTISGLQAEDEADYYCSSYTSSSTRV
FGTGTKVTVL.

In a specific aspect, the sequence identity is 86%, 87%, 88%, 89%, 90%, 91%, 92%, 93%, 94%, 95%, 96%, 97%, 98%, 99% or 100%.

In another embodiment the antibody binds to human, mouse, or cynomolgus monkey PD-L1. In a specific aspect the antibody is capable of blocking the interaction between human, mice, or cynomolgus monkey PD-L1 and the respective human, mouse, or cynomolgus monkey PD-1 receptors.

In another embodiment, the antibody binds to human PD-L1 with a KD of 5×10⁻⁹ M or less, preferably with a KD of 2×10⁻⁹ M or less, and even more preferred with a KD of 1×10⁻⁹ M or less.

In yet another embodiment, the disclosure relates to an anti-PD-L1 antibody or antigen binding fragment thereof which binds to a functional epitope including residues Y56 and D61 of human PD-L1.

In a specific aspect, the functional epitope further includes E58, E60, Q66, R113, and M115 of human PD-L1.

In a more specific aspect, the antibody binds to a conformational epitope, including residues 54-66 and 112-122 of human PD-L1.

In certain embodiments, the disclosure is related to an anti-PD-L1 antibody, or antigen binding fragment thereof, which cross-competes for binding to PD-L1 with an antibody according to the disclosure as described herein.

In certain embodiments, the disclosure features proteins and polypeptides including any of the above described anti-PD-L1 antibodies in combination with at least one pharmaceutically acceptable carrier.

In certain embodiments, the disclosure features an isolated nucleic acid encoding a polypeptide, or light chain or a heavy chain variable region sequence of an anti-PD-L1 antibody, or antigen binding fragment thereof, as described herein. In certain embodiments, the disclosure provides for an isolated nucleic acid encoding a light chain or a heavy chain variable region sequence of an anti-PD-L1 antibody, wherein:

(a) the heavy chain includes an HVR-H1, an HVR-H2, and an HVR-H3 sequence having at least 80% sequence identity to SYIMM (SEQ ID NO: 35), SIYPSGGITFYADTVKG (SEQ ID NO: 36), and IKLGTVTTVDY (SEQ ID NO: 37), respectively, or

(b) the light chain includes an HVR-L1, an HVR-L2, and an HVR-L3 sequence having at least 80% sequence identity to TGTSSDVGGYNYVS (SEQ ID NO: 38), DVSNRPS (SEQ ID NO: 39), and SSYTSSSTRV (SEQ ID NO: 40), respectively.

In a specific aspect, the sequence identity is 81%, 82%, 83%, 84%, 85%, 86%, 87%, 88%, 89%, 90%, 91%, 92%, 93%, 94%, 95%, 96%, 97%, 98%, 99%, or 100%.

In a further aspect, the nucleic acid sequence for the heavy chain is:

| (SEQ ID NO: 48) | |
|---|---|
| atggagttgc ctgttaggct gttggtgctg atgttctgga ttcctgctag ctccagcgag | 60 |
| gtgcagctgc tggaatccgg cggaggactg gtgcagcctg gcggctccct gagactgtct | 120 |
| tgcgccgcct ccggcttcac cttctccagc tacatcatga tgtgggtgcg acaggcccct | 180 |
| ggcaagggcc tggaatgggt gtcctccatc tacccctccg gcggcatcac cttctacgcc | 240 |
| gacaccgtga agggccggtt caccatctcc cgggacaact ccaagaacac cctgtacctg | 300 |
| cagatgaact ccctgcgggc cgaggacacc gccgtgtact actgcgcccg gatcaagctg | 360 |
| ggcaccgtga ccaccgtgga ctactggggc cagggcaccc tggtgacagt gtcctccgcc | 420 |
| tccaccaagg gcccatcggt cttccccctg gcaccctcct ccaagagcac ctctgggggc | 480 |
| acagcggccc tgggctgcct ggtcaaggac tacttccccg aaccggtgac ggtgtcgtgg | 540 |
| aactcaggcg ccctgaccag cggcgtgcac accttcccgg ctgtcctaca gtcctcagga | 600 |
| ctctactccc tcagcagcgt ggtgaccgtg ccctccagca gcttgggcac ccagacctac | 660 |
| atctgcaacg tgaatcacaa gcccagcaac accaaggtgg acaagaaagt tgagcccaaa | 720 |
| tcttgtgaca aaactcacac atgcccaccg tgcccagcac ctgaactcct ggggggaccg | 780 |
| tcagtcttcc tcttcccccc aaaacccaag gacaccctca tgatctcccg gacccctgag | 840 |
| gtcacatgcg tggtggtgga cgtgagccac gaagaccctg aggtcaagtt caactggtac | 900 |
| gtggacggcg tggaggtgca taatgccaag acaaagccgc gggaggagca gtacaacagc | 960 |
| acgtaccgtg tggtcagcgt cctcaccgtc ctgcaccagg actggctgaa tggcaaggag | 1020 |
| tacaagtgca aggtctccaa caaagccctc ccagccccca tcgagaaaac catctccaaa | 1080 |
| gccaaagggc agccccgaga accacaggtg tacaccctgc ccccatcacg ggatgagctg | 1140 |
| accaagaacc aggtcagcct gacctgcctg gtcaaaggct tctatcccag cgacatcgcc | 1200 |
| gtggagtggg agagcaatgg gcagccggag aacaactaca agaccacgcc tcccgtgctg | 1260 |
| gactccgacg gctccttctt cctctatagc aagctcaccg tggacaagag caggtggcag | 1320 |
| caggggaacg tcttctcatg ctccgtgatg catgaggctc tgcacaacca ctacacgcag | 1380 |
| aagagcctct ccctgtcccc gggtaaa | 1407 |

and the nucleic acid sequence for the light chain is:

| (SEQ ID NO: 49) | |
|---|---|
| atggagttgc ctgttaggct gttggtgctg atgttctgga ttcctgcttc cttaagccag | 60 |
| tccgccctga cccagcctgc ctccgtgtct ggctcccctg gccagtccat caccatcagc | 120 |
| tgcaccggca cctccagcga cgtgggcggc tacaactacg tgtcctggta tcagcagcac | 180 |
| cccggcaagg cccccaagct gatgatctac gacgtgtcca accggccctc cggcgtgtcc | 240 |
| aacagattct ccggctccaa gtccggcaac accgcctccc tgaccatcag cggactgcag | 300 |
| gcagaggacg aggccgacta ctactgctcc tcctacacct cctccagcac cagagtgttc | 360 |
| ggcaccggca caaaagtgac cgtgctgggc cagcccaagg ccaacccaac cgtgacactg | 420 |
| ttccccccat cctccgagga actgcaggcc aacaaggcca ccctggtctg cctgatctca | 480 |
| aatttctatc caggcgccgt gaccgtggcc tggaaggctg atggctcccc agtgaaggcc | 540 |
| ggcgtggaaa ccaccaagcc ctccaagcag tccaacaaca aatacgccgc ctcctcctac | 600 |
| ctgtccctga cccccgagca gtggaagtcc caccggtcct acagctgcca ggtcacacac | 660 |
| gagggctcca ccgtggaaaa gaccgtcgcc cccaccgagt gctca. | 705 |

Further exemplary anti-PD-L1 antibodies that can be used in an anti-PD-L1/TGFβ Trap are described in US patent application publication US 2010/0203056. In one embodiment of the disclosure, the antibody moiety is YW243.55570. In another embodiment of the disclosure, the antibody moiety is MPDL3289A.

In certain embodiments, the disclosure features an anti-PD-L1 antibody moiety including a heavy chain and a light chain variable region sequence, where:

(a) the heavy chain sequence has at least 85% sequence identity to the heavy chain sequence:

(SEQ ID NO: 12)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS,

and

(b) the light chain sequence has at least 85% sequence identity to the light chain sequence:

(SEQ ID NO: 13)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR

In certain embodiments, the disclosure features an anti-PD-L1 antibody moiety including a heavy chain and a light chain variable region sequence, where:

(a) the heavy chain sequence has at least 85% sequence identity to the heavy chain sequence:

(SEQ ID NO: 14)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSA,

and

(b) the light chain sequence has at least 85% sequence identity to the light chain sequence:

(SEQ ID NO: 13)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR

Yet further exemplary anti-PD-L1 antibodies that can be used in an anti-PD-L1/TGFβ Trap are described in US patent publication U.S. Pat. No. 7,943,743.

In one embodiment of the disclosure, the anti-PD-L1 antibody is MDX-1105.

In certain embodiments, the anti-PD-L1 antibody is MEDI-4736.

### Constant Region

The proteins and peptides of the disclosure can include a constant region of an immunoglobulin or a fragment, analog, variant, mutant, or derivative of the constant region. In certain embodiments, the constant region is derived from a human immunoglobulin heavy chain, for example, IgG1, IgG2, IgG3, IgG4, or other classes. In certain embodiments, the constant region includes a CH2 domain. In certain embodiments, the constant region includes CH2 and CH3 domains or includes hinge-CH2-CH3. Alternatively, the constant region can include all or a portion of the hinge region, the CH2 domain and/or the CH3 domain.

In one embodiment, the constant region contains a mutation that reduces affinity for an Fc receptor or reduces Fc effector function. For example, the constant region can contain a mutation that eliminates the glycosylation site within the constant region of an IgG heavy chain. In some embodiments, the constant region contains mutations, deletions, or insertions at an amino acid position corresponding to Leu234, Leu235, Gly236, Gly237, Asn297, or Pro331 of IgG1 (amino acids are numbered according to EU nomenclature). In a particular embodiment, the constant region contains a mutation at an amino acid position corresponding to Asn297 of IgG1. In alternative embodiments, the constant region contains mutations, deletions, or insertions at an amino acid position corresponding to Leu281, Leu282, Gly283, Gly284, Asn344, or Pro378 of IgG1.

In some embodiments, the constant region contains a CH2 domain derived from a human IgG2 or IgG4 heavy chain. Preferably, the CH2 domain contains a mutation that eliminates the glycosylation site within the CH2 domain. In one embodiment, the mutation alters the asparagine within the Gln-Phe-Asn-Ser (SEQ ID NO: 15) amino acid sequence within the CH2 domain of the IgG2 or IgG4 heavy chain. Preferably, the mutation changes the asparagine to a glutamine. Alternatively, the mutation alters both the phenylalanine and the asparagine within the Gln-Phe-Asn-Ser (SEQ ID NO: 15) amino acid sequence. In one embodiment, the Gln-Phe-Asn-Ser (SEQ ID NO: 15) amino acid sequence is replaced with a Gln-Ala-Gln-Ser (SEQ ID NO: 16) amino acid sequence. The asparagine within the Gln-Phe-Asn-Ser (SEQ ID NO: 15) amino acid sequence corresponds to Asn297 of IgG1.

In another embodiment, the constant region includes a CH2 domain and at least a portion of a hinge region. The hinge region can be derived from an immunoglobulin heavy chain, e.g., IgG1, IgG2, IgG3, IgG4, or other classes. Preferably, the hinge region is derived from human IgG1, IgG2, IgG3, IgG4, or other suitable classes. More preferably the hinge region is derived from a human IgG1 heavy chain. In one embodiment the cysteine in the Pro-Lys-Ser-Cys-Asp-Lys (SEQ ID NO: 17) amino acid sequence of the IgG1 hinge region is altered. In certain embodiments, the Pro-Lys-Ser-Cys-Asp-Lys (SEQ ID NO: 17) amino acid sequence is replaced with a Pro-Lys-Ser-Ser-Asp-Lys (SEQ ID NO: 18) amino acid sequence. In certain embodiments, the constant region includes a CH2 domain derived from a first antibody isotype and a hinge region derived from a second antibody isotype. In certain embodiments, the CH2 domain is derived from a human IgG2 or IgG4 heavy chain, while the hinge region is derived from an altered human IgG1 heavy chain.

The alteration of amino acids near the junction of the Fc portion and the non-Fc portion can dramatically increase the serum half-life of the Fc fusion protein (PCT publication WO 0158957, the disclosure of which is hereby incorporated by reference). Accordingly, the junction region of a protein or polypeptide of the present disclosure can contain alterations that, relative to the naturally-occurring sequences of an immunoglobulin heavy chain and erythropoietin, preferably lie within about 10 amino acids of the junction point. These amino acid changes can cause an increase in hydrophobicity. In one embodiment, the constant region is derived from an IgG sequence in which the C-terminal lysine residue is replaced. Preferably, the C-terminal lysine of an IgG sequence is replaced with a non-lysine amino acid, such as alanine or leucine, to further increase serum half-life. In another embodiment, the constant region is derived from an IgG sequence in which the Leu-Ser-Leu-Ser (SEQ ID NO: 19) amino acid sequence near the C-terminus of the constant region is altered to eliminate potential junctional T-cell epitopes. For example, in one embodiment, the Leu-Ser-Leu-Ser (SEQ ID NO: 19) amino acid sequence is replaced with an Ala-Thr-Ala-Thr (SEQ ID NO: 20) amino acid sequence. In other embodiments, the amino acids within the Leu-Ser-Leu-Ser (SEQ ID NO: 19) segment are replaced with other amino acids such as glycine or proline. Detailed methods of generating amino acid substitutions of the Leu-Ser-Leu-Ser (SEQ ID NO: 19) segment near the C-terminus of an IgG1, IgG2, IgG3, IgG4, or other immunoglobulin class molecule have been described in U.S. Patent Publication No. 20030166877, the disclosure of which is hereby incorporated by reference.

Suitable hinge regions for the present disclosure can be derived from IgG1, IgG2, IgG3, IgG4, and other immunoglobulin classes. The IgG1 hinge region has three cysteines, two of which are involved in disulfide bonds between the two heavy chains of the immunoglobulin. These same cysteines permit efficient and consistent disulfide bonding formation between Fc portions. Therefore, a hinge region of the present disclosure is derived from IgG1, e.g., human IgG1. In some embodiments, the first cysteine within the human IgG1 hinge region is mutated to another amino acid, preferably serine. The IgG2 isotype hinge region has four disulfide bonds that tend to promote oligomerization and possibly incorrect disulfide bonding during secretion in recombinant systems. A suitable hinge region can be derived from an IgG2 hinge; the first two cysteines are each preferably mutated to another amino acid. The hinge region of IgG4 is known to form interchain disulfide bonds inefficiently. However, a suitable hinge region for the present disclosure can be derived from the IgG4 hinge region, preferably containing a mutation that enhances correct formation of disulfide bonds between heavy chain-derived moieties (Angal S, et al. (1993) Mol. Immunol., 30:105-8).

In accordance with the present disclosure, the constant region can contain CH2 and/or CH3 domains and a hinge region that are derived from different antibody isotypes, i.e., a hybrid constant region. For example, in one embodiment, the constant region contains CH2 and/or CH3 domains derived from IgG2 or IgG4 and a mutant hinge region derived from IgG1. Alternatively, a mutant hinge region from another IgG subclass is used in a hybrid constant region. For example, a mutant form of the IgG4 hinge that allows efficient disulfide bonding between the two heavy chains can be used. A mutant hinge can also be derived from an IgG2 hinge in which the first two cysteines are each mutated to another amino acid. Assembly of such hybrid constant regions has been described in U.S. Patent Publication No. 20030044423, the disclosure of which is hereby incorporated by reference.

In accordance with the present disclosure, the constant region can contain one or more mutations described herein. The combinations of mutations in the Fc portion can have additive or synergistic effects on the prolonged serum half-life and increased in vivo potency of the bifunctional molecule. Thus, in one exemplary embodiment, the constant region can contain (i) a region derived from an IgG sequence in which the Leu-Ser-Leu-Ser (SEQ ID NO: 19) amino acid sequence is replaced with an Ala-Thr-Ala-Thr (SEQ ID NO: 20) amino acid sequence; (ii) a C-terminal alanine residue instead of lysine; (iii) a CH2 domain and a hinge region that are derived from different antibody isotypes, for example, an IgG2 CH2 domain and an altered IgG1 hinge region; and (iv) a mutation that eliminates the glycosylation site within the IgG2-derived CH2 domain, for example, a Gln-Ala-Gln-Ser (SEQ ID NO: 16) amino acid sequence instead of the Gln-Phe-Asn-Ser (SEQ ID NO: 15) amino acid sequence within the IgG2-derived CH2 domain.

### Antibody Fragments

The proteins and polypeptides of the disclosure can also include antigen-binding fragments of antibodies. Exemplary antibody fragments include scFv, Fv, Fab, F(ab′)₂, and single domain VHH fragments such as those of camelid origin.

Single-chain antibody fragments, also known as single-chain antibodies (scFvs), are recombinant polypeptides which typically bind antigens or receptors; these fragments contain at least one fragment of an antibody variable heavy-chain amino acid sequence (V_{H}) tethered to at least one fragment of an antibody variable light-chain sequence (V_{L}) with or without one or more interconnecting linkers. Such a linker may be a short, flexible peptide selected to assure that the proper three-dimensional folding of the V_{L} and V_{H} domains occurs once they are linked so as to maintain the target molecule binding-specificity of the whole antibody from which the single-chain antibody fragment is derived. Generally, the carboxyl terminus of the V_{L} or V_{H} sequence is covalently linked by such a peptide linker to the amino acid terminus of a complementary V_{L} and V_{H} sequence. Single-chain antibody fragments can be generated by molecular cloning, antibody phage display library or similar techniques. These proteins can be produced either in eukaryotic cells or prokaryotic cells, including bacteria.

Single-chain antibody fragments contain amino acid sequences having at least one of the variable regions or CDRs of the whole antibodies described in this specification, but are lacking some or all of the constant domains of those antibodies. These constant domains are not necessary for antigen binding, but constitute a major portion of the structure of whole antibodies. Single-chain antibody fragments may therefore overcome some of the problems associated with the use of antibodies containing part or all of a constant domain. For example, single-chain antibody fragments tend to be free of undesired interactions between biological molecules and the heavy-chain constant region, or other unwanted biological activity. Additionally, single-chain antibody fragments are considerably smaller than whole antibodies and may therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely than whole antibodies to provoke an immune response in a recipient.

Fragments of antibodies that have the same or comparable binding characteristics to those of the whole antibody may also be present. Such fragments may contain one or both Fab fragments or the F(ab′)₂ fragment. The antibody fragments may contain all six CDRs of the whole antibody, although fragments containing fewer than all of such regions, such as three, four or five CDRs, are also functional.

### Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In one aspect, the present disclosure provides an intravenous drug delivery formulation that includes 500 mg-2000 mg of a protein including a first polypeptide and a second polypeptide, the first polypeptide includes: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ), the second polypeptide includes at least a variable region of a light chain of an antibody that binds PD-L1, and the heavy chain of the first polypeptide and the light chain of the second polypeptide, when combined, form an antigen binding site that binds PD-L1.

In certain embodiments, a protein product of the present disclosure includes a first polypeptide that includes the amino acid sequence of SEQ ID NO: 3, and a second polypeptide that includes the amino acid sequence of SEQ ID NO: 1.

In certain embodiments of the present disclosure, the intravenous drug delivery formulation may include about 500 mg to about 2400 mg dose (e.g., about 500 mg to about 2300 mg, about 500 mg to about 2200 mg, about 500 mg to about 2100 mg, about 500 mg to about 2000 mg, about 500 mg to about 1900 mg, about 500 mg to about 1800 mg, about 500 mg to about 1700 mg, about 500 mg to about 1600 mg, about 500 mg to about 1500 mg, about 500 mg to about 1400 mg, about 500 mg to about 1300 mg, about 500 mg to about 1200 mg, about 500 mg to about 1100 mg, about 500 mg to about 1000 mg, about 500 mg to about 900 mg, about 500 mg to about 800 mg, about 500 mg to about 700 mg, about 500 mg to about 600 mg, about 600 mg to 2400 mg, about 700 mg to 2400 mg, about 800 mg to 2400 mg, about 900 mg to 2400 mg, about 1000 mg to 2400 mg, about 1100 mg to 2400 mg, about 1200 mg to 2400 mg, about 1300 mg to 2400 mg, about 1400 mg to 2400 mg, about 1500 mg to 2400 mg, about 1600 mg to 2400 mg, about 1700 mg to 2400 mg, about 1800 mg to 2400 mg, about 1900 mg to 2400 mg, about 2000 mg to 2400 mg, about 2100 mg to 2400 mg, about 2200 mg to 2400 mg, or about 2300 mg to 2400 mg) of a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)). In certain embodiments, the intravenous drug delivery formulation may include about 500 to about 2000 mg dose of a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)). In certain embodiments, the intravenous drug delivery formulation may include about 500 mg dose of a protein product of the present disclosure with a first polypeptide that includes the amino acid sequence of SEQ ID NO: 3, and the second polypeptide that includes the amino acid sequence of SEQ ID NO: 1. In certain embodiments, the intravenous drug delivery formulation may include 500 mg dose of a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)). In certain embodiments, the intravenous drug delivery formulation may include about 1200 mg dose of a protein product of the present disclosure with a first polypeptide that includes the amino acid sequence of SEQ ID NO: 3, and the second polypeptide that includes the amino acid sequence of SEQ ID NO: 1. In certain embodiments, the intravenous drug delivery formulation may include 1200 mg dose of a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)). In certain embodiments, the intravenous drug delivery formulation may include about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg) of a protein product of the present disclosure (e.g., anti-PD-L1/TGFβ Trap). In certain embodiments, the intravenous drug delivery formulation may include about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg) of a protein product with a first polypeptide that includes the amino acid sequence of SEQ ID NO: 3, and the second polypeptide that includes the amino acid sequence of SEQ ID NO: 1.

In certain embodiments, the intravenous drug delivery formulation may include about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2100 mg, about 2200 mg, about 2300 mg, or about 2400 mg of the protein of the present disclosure (e.g., anti-PD-L1/TGFβ Trap).

The intravenous drug delivery formulation of the present disclosure may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may freeze-dried (lyophilized) and contained in about 12-60 vials. In certain embodiments, the formulation may be freeze-dried and about 45 mg of the freeze-dried formulation may be contained in one vial. In certain embodiments, the about 40 mg-about 100 mg of freeze-dried formulation may be contained in one vial. In certain embodiments, freeze dried formulation from 12, 27, or 45 vials are combined to obtained a therapeutic dose of the protein in the intravenous drug formulation. In certain embodiments, the formulation may be a liquid formulation of a protein product with a first polypeptide that includes the amino acid sequence of SEQ ID NO: 3, and the second polypeptide that includes the amino acid sequence of SEQ ID NO: 1, and stored as about 250 mg/vial to about 2000 mg/vial (e.g., about 250 mg/vial to about 2000 mg/vial, about 250 mg/vial to about 1900 mg/vial, about 250 mg/vial to about 1800 mg/vial, about 250 mg/vial to about 1700 mg/vial, about 250 mg/vial to about 1600 mg/vial, about 250 mg/vial to about 1500 mg/vial, about 250 mg/vial to about 1400 mg/vial, about 250 mg/vial to about 1300 mg/vial, about 250 mg/vial to about 1200 mg/vial, about 250 mg/vial to about 1100 mg/vial, about 250 mg/vial to about 1000 mg/vial, about 250 mg/vial to about 900 mg/vial, about 250 mg/vial to about 800 mg/vial, about 250 mg/vial to about 700 mg/vial, about 250 mg/vial to about 600 mg/vial, about 250 mg/vial to about 500 mg/vial, about 250 mg/vial to about 400 mg/vial, about 250 mg/vial to about 300 mg/vial, about 300 mg/vial to about 2000 mg/vial, about 400 mg/vial to about 2000 mg/vial, about 500 mg/vial to about 2000 mg/vial, about 600 mg/vial to about 2000 mg/vial, about 700 mg/vial to about 2000 mg/vial, about 800 mg/vial to about 2000 mg/vial, about 900 mg/vial to about 2000 mg/vial, about 1000 mg/vial to about 2000 mg/vial, about 1100 mg/vial to about 2000 mg/vial, about 1200 mg/vial to about 2000 mg/vial, about 1300 mg/vial to about 2000 mg/vial, about 1400 mg/vial to about 2000 mg/vial, about 1500 mg/vial to about 2000 mg/vial, about 1600 mg/vial to about 2000 mg/vial, about 1700 mg/vial to about 2000 mg/vial, about 1800 mg/vial to about 2000 mg/vial, or about 1900 mg/vial to about 2000 mg/vial). In certain embodiments, the formulation may be a liquid formulation and stored as about 600 mg/vial. In certain embodiments, the formulation may be a liquid formulation and stored as about 1200 mg/vial. In certain embodiments, the formulation may be a liquid formulation and stored as about 1800 mg/vial. In certain embodiments, the formulation may be a liquid formulation and stored as about 250 mg/vial.

This disclosure provides a liquid aqueous pharmaceutical formulation including a therapeutically effective amount of the protein of the present disclosure (e.g., anti-PD-L1/TGFβ Trap) in a buffered solution forming a formulation.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)), in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including a protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)) in a pH-buffered solution. The buffer of this invention may have a pH ranging from about 4 to about 8, e.g., from about 4 to about 8, from about 4.5 to about 8, from about 5 to about 8, from about 5.5 to about 8, from about 6 to about 8, from about 6.5 to about 8, from about 7 to about 8, from about 7.5 to about 8, from about 4 to about 7.5, from about 4.5 to about 7.5, from about 5 to about about 7.5, from about 5.5 to about 7.5, from about 6 to about 7.5, from about 6.5 to about 7.5, from about 4 to about 7, from about 4.5 to about 7, from about 5 to about 7, from about 5.5 to about 7, from about 6 to about 7, from about 4 to about 6.5, from about 4.5 to about 6.5, from about 5 to about 6.5, from about 5.5 to about 6.5, from about 4 to about 6.0, from about 4.5 to about 6.0, from about 5 to about 6, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g. sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/ml of citric acid (e.g., 1.305 mg/ml), about 0.3 mg/ml of sodium citrate (e.g., 0.305 mg/ml), about 1.5 mg/ml of disodium phosphate dihydrate (e.g., 1.53 mg/ml), about 0.9 mg/ml of sodium dihydrogen phosphate dihydrate (e.g., 0.86), and about 6.2 mg/ml of sodium chloride (e.g., 6.165 mg/ml). In certain embodiments, the buffer system includes about 1-1.5 mg/ml of citric acid, about 0.25 to about 0.5 mg/ml of sodium citrate, about 1.25 to about 1.75 mg/ml of disodium phosphate dihydrate, about 0.7 to about 1.1 mg/ml of sodium dihydrogen phosphate dihydrate, and 6.0 to 6.4 mg/ml of sodium chloride. In certain embodiments, the pH of the formulation is adjusted with sodium hydroxide.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also alter with respect to the molecular weight of the polyol. For example, a lower amount of a monosaccharide (e.g. mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/ml. In certain embodiments, the concentration of mannitol may be about 7.5 to about 15 mg/ml. In certain embodiments, the concentration of mannitol may be about 10-about 14 mg/ml. In certain embodiments, the concentration of mannitol may be about 12 mg/ml. In certain embodiments, the polyol sorbitol may be included in the formulation.

A detergent or surfactant may also be added to the formulation. Exemplary detergents include nonionic detergents such as polysorbates (e.g. polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hilfsstoffe, Editio Cantor Verlag Aulendorf, 4th edi., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

### Lyophilized Formulation

The lyophilized formulation of the present disclosure includes the anti-PD-L1/TGFβ Trap molecule and a lyoprotectant. The lyoprotectant may be sugar, e.g., disaccharides. In certain embodiments, the lycoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing the protein of the present disclosure may be adjusted between about 6 to about 8. In certain embodiments, the pH range for the lyophilized drug product may be from about 7 to about 8.

In certain embodiments, a salt or buffer components may be added in an amount of about 10 mM-about 200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

A preservative may be optionally added to the formulations herein to reduce bacterial action. The addition of a preservative may, for example, facilitate the production of a multi-use (multiple-dose) formulation.

In certain embodiments, the lyophilized drug product may be constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g. phosphate-buffered saline), sterile saline solution, Ringer's solution or dextrose solution.

In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution.

In certain embodiments, the lyophilized protein product of the instant disclosure is constituted to about 4.5 mL water for injection and diluted with 0.9% saline solution (sodium chloride solution).

### Liquid Formulation

In embodiments, the protein product of the present disclosure is formulated as a liquid formulation. The liquid formulation may be presented at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments vials may be filled with about 61.2 mL of the protein product solution in order to allow an extractable volume of 60 mL. In certain embodiments, the liquid formulation may be diluted with 0.9% saline solution. In certain embodiments vials may contain about 61.2 mL of the protein product (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)) solution of about 20 mg/mL to about 50 mg/mL (e.g., about 20 mg/mL, about 25 mg/mL, about 30 mg/mL, about 35 mg/mL, about 40 mg/mL, about 45 mg/mL or about 50 mg/mL) in order to allow an extractable volume of 60 mL for delivering about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg) of the protein product (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)) to a subject.

In certain embodiments vials may contain about 61.2 mL of the protein product solution (protein product with a first polypeptide that includes the amino acid sequence of SEQ ID NO: 3, and the second polypeptide that includes the amino acid sequence of SEQ ID NO: 1) of about 20 mg/mL to about 50 mg/mL (e.g., about 20 mg/mL, about 25 mg/mL, about 30 mg/mL, about 35 mg/mL, about 40 mg/mL, about 45 mg/mL or about 50 mg/mL) in order to allow an extractable volume of 60 mL for delivering about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg) of the protein product to a subject.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In addition to aggregation, deamidation is a common product variant of peptides and proteins that may occur during fermentation, harvest/cell clarification, purification, drug substance/drug product storage and during sample analysis. Deamidation is the loss of NH₃ from a protein forming a succinimide intermediate that can undergo hydrolysis. The succinimide intermediate results in a 17 u mass decrease of the parent peptide. The subsequent hydrolysis results in an 18 u mass increase. Isolation of the succinimide intermediate is difficult due to instability under aqueous conditions. As such, deamidation is typically detectable as 1 u mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation.

In certain embodiments, the liquid formulation of the present disclosure may be preserved under conditions of pH and humidity to prevent deamination of the protein product.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g. phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

Intravenous (IV) formulations may be the preferred administration route in particular instances, such as when a patient is in the hospital after transplantation receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

A preservative may be optionally added to the formulations herein to reduce bacterial action. The addition of a preservative may, for example, facilitate the production of a multi-use (multiple-dose) formulation.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g. phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

### Method of Treating Cancer or Inhibiting Tumor Growth

In one aspect the present disclosure provides a method of treating cancer or inhibiting tumor growth in a subject in need thereof, the method including administering to the subject a dose of at least 500 mg of a protein including a first polypeptide and a second polypeptide. The first polypeptide includes: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ). The second polypeptide includes at least a variable region of a light chain of an antibody that binds PD-L1, and the heavy chain of the first polypeptide and the light chain of the second polypeptide, when combined, form an antigen binding site that binds PD-L1.

In certain embodiments, the method of treating cancer or inhibiting tumor growth of the present disclosure involves administering to a subject a protein including two peptides in which the first polypeptide includes the amino acid sequence of SEQ ID NO: 3, and the second polypeptide includes the amino acid sequence of SEQ ID NO: 1. In certain embodiments, the protein is an anti-PD-L1/TGFβ Trap molecule.

In certain embodiments, the method of treating cancer or inhibiting tumor growth of the present disclosure involves administering to a subject a protein (e.g., an anti-PD-L1/TGFβ Trap molecule (e.g., including a first polypeptide that includes the amino acid sequence of SEQ ID NO: 3, and a second polypeptide that includes the amino acid sequence of SEQ ID NO: 1)) at a dose of about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200, about 1300, about 1400, about 1500, about 1600, about 1700, about 1800, about 1900, about 2000, about 2100, about 2200, about 2300, about 2400, about 2500 mg, about 2600 mg, about 2700 mg, about 2800 mg, about 2900 mg, or about 3000 mg). In certain embodiments, about 1200 mg of anti-PD-L1/TGFβ Trap molecule is administered to a subject once every two weeks. In certain embodiments, about 1800 mg of anti-PD-L1/TGFβ Trap molecule is administered to a subject once every three weeks. In certain embodiments, about 1200 mg of a protein product with a first polypeptide that includes the amino acid sequence of SEQ ID NO: 3 and the second polypeptide that includes the amino acid sequence of SEQ ID NO: 1 is administered to a subject once every two weeks. In certain embodiments, about 1800 mg of a protein product with a first polypeptide that includes the amino acid sequence of SEQ ID NO: 3 and the second polypeptide that includes the amino acid sequence of SEQ ID NO: 1 is administered to a subject once every three weeks.

In certain embodiments, the dose may be about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, 1875 mg, about 1900 mg, about 1925 mg, about 1950 mg, about 1975 mg, about 2000 mg, 2100 mg, about 2200 mg, about 2300 mg, or about 2400 mg.

In certain embodiments, the dose may be administered once every two weeks. In certain embodiments, the protein may be administered by intravenous administration, e.g., with a prefilled bag, a prefilled pen, or a prefilled syringes. In certain embodiments, the protein is administered intravenously from a 250 ml saline bag, and the intravenous infusion may be for about one hour (e.g., 50 to 80 minutes). In certain embodiments, the bag is connected to a channel comprising a tube and/or a needle.

In certain embodiments, the method treats a cancer or inhibits tumor growth, for example, among the following: non-small cell lung cancer, melanoma, pancreatic cancer, colorectal cancer, ovarian cancer, breast cancer, prostate cancer, glioblastoma, gastric cancer, biliary tract cancer, esophageal cancer (squamous cell carcinoma or adenocarcinoma), adenoma of the head or the neck, squamous carcinoma of the head or the neck, prostate cancer, renal cancer, cervical cancer, myeloma, lymphoma, leukemia, thyroid cancer, endometrial cancer, uterine cancer, bladder cancer, neuroendocrine cancer, liver cancer, nasopharyngeal cancer, testicular cancer, small cell lung cancer, basal cell skin cancer, squamous cell skin cancer, dermatofibrosarcoma protuberans, Merkel cell carcinoma, glioma, sarcoma, mesothelioma, and myelodysplastic syndromes. In certain embodiments, the method treats a cancer of pretreated patients, for example pretreated non-small cell lung cancer, pretreated melanoma, pretreated pancreatic cancer, pretreated colorectal cancer, pretreated ovarian cancer, pretreated breast cancer, pretreated glioblastoma, pretreated recurrent or refractory unresectable Stage IV gastric cancer, pretreated biliary tract cancer, pretreated esophageal cancer (squamous cell carcinoma or adenocarcinoma), pretreated adenoma of the head or the neck, pretreated squamous carcinoma of the head or the neck, pretreated prostate cancer, pretreated renal cancer, pretreated cervical cancer, pretreated myeloma, pretreated lymphoma, pretreated leukemia, pretreated thyroid cancer, pretreated endometrial cancer, pretreated uterine cancer, pretreated bladder cancer, pretreated neuroendocrine cancer, pretreated liver cancer, pretreated nasopharyngeal cancer, pretreated testicular cancer, pretreated small cell lung cancer, pretreated basal cell skin cancer, pretreated squamous cell skin cancer, pretreated dermatofibrosarcoma protuberans, pretreated Merkel cell carcinoma, pretreated glioma, pretreated sarcoma, pretreated mesothelioma, and pretreated myelodysplastic syndromes.

In certain embodiments, the tumor is an advanced solid tumor. In certain embodiments, the tumor is refractory to prior treatment. In certain embodiments, patients who had advanced NSCLC and were previously treated with anti-PD-1 or anti-PD-L1 agent (“PDx therapy”) and subsequently had documented disease progression are treated by intravenously administering about 1200 mg of anti-PD-L1/TGFβ Trap. Patient best overall response (BOR) to prior PDx therapy was documented. In certain embodiments, patients with progressive disease (PD) following prior PDx therapy, thus considered as primary refractory, (i.e., among these patients disease progression was observed following PDx therapy initiation without any observed benefit from the treatment) are treated by intravenously administering about 1200 mg-about 2400 mg (e.g., about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 2400 mg, about 1400 mg to about 2400 mg, about 1500 mg to about 2400 mg, about 1600 mg to about 2400 mg, about 1700 mg to about 2400 mg, about 1800 mg to about 2400 mg, about 1900 mg to about 2400 mg, about 2000 mg to about 2400 mg, about 2100 mg to about 2400 mg, about 2200 mg to about 2400 mg, or about 2300 mg to about 2400 mg) of anti-PD-L1/TGFβ Trap. In certain embodiments, patients characterized as acquired resistant, i.e., the patients' disease initially responded to prior PDx therapy but the patients ultimately reverted to disease progression stage, are treated by intravenously administering about 1200 mg to about 2400 mg of anti-PD-L1/TGFβ Trap, which includes a first polypeptide that includes the amino acid sequence of SEQ ID NO: 3, and a second polypeptide that includes the amino acid sequence of SEQ ID NO: 1. These patients' BOR to prior PDx therapy was stable disease (SD), partial response (PR), or complete response (CR), who then experienced subsequent disease progression. The rationale for using anti-PD-L1/TGFβ Trap in these NSCLC PDx-fail sub-cohorts is to also neutralize TGF-β, a molecule known to inhibit tumor immune activation, for stimulating clinical response in patients who failed to respond to prior PDx therapy alone.

In certain embodiments, patients who had advanced NSCLC with refractory, relapsed or progressive disease on or after a single line of platinum-based chemtherapy are treated by intravenously administering about 1200 mg-about 2400 mg (e.g., about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 2400 mg, about 1400 mg to about 2400 mg, about 1500 mg to about 2400 mg, about 1600 mg to about 2400 mg, about 1700 mg to about 2400 mg, about 1800 mg to about 2400 mg, about 1900 mg to about 2400 mg, about 2000 mg to about 2400 mg, about 2100 mg to about 2400 mg, about 2200 mg to about 2400 mg, or about 2300 mg to about 2400 mg) of anti-PD-L1/TGFβ Trap, which includes a first polypeptide that includes the amino acid sequence of SEQ ID NO: 3, and a second polypeptide that includes the amino acid sequence of SEQ ID NO: 1. In certain embodiments, patients who had advanced NSCLC with refractory, relapsed or progressive disease on or after a single line of platinum-based chemtherapy are treated by intravenously administering anti-PD-L1/TGFβ Trap at a dose of about 1200 mg once every 2 weeks. In certain embodiments, patients who had advanced NSCLC with refractory, relapsed or progressive disease on or after a single line of platinum-based chemtherapy are treated by intravenously administering anti-PD-L1/TGFβ Trap at a dose of about 500 mg-about 1200 mg (e.g., about 500 mg to about 1000 mg, about 500 mg to about 1000 mg, about 500 mg to about 900 mg, about 500 mg to about 800 mg, about 500 mg to about 700 mg, about 500 mg to about 600 mg) which includes a first polypeptide that includes the amino acid sequence of SEQ ID NO: 3, and a second polypeptide that includes the amino acid sequence of SEQ ID NO: 1. In certain embodiments, patients who had advanced NSCLC with refractory, relapsed or progressive disease on or after a single line of platinum-based chemtherapy are treated by intravenously administering anti-PD-L1/TGFβ Trap at a dose of about 500 mg once every 2 weeks.

In certain embodiments, patients with heavily pretreated recurrent or refractory unresectable Stage IV gastric cancer are treated by intravenously administering about 1200 mg-about 2400 mg (e.g., about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 2400 mg, about 1400 mg to about 2400 mg, about 1500 mg to about 2400 mg, about 1600 mg to about 2400 mg, about 1700 mg to about 2400 mg, about 1800 mg to about 2400 mg, about 1900 mg to about 2400 mg, about 2000 mg to about 2400 mg, about 2100 mg to about 2400 mg, about 2200 mg to about 2400 mg, or about 2300 mg to about 2400 mg) of anti-PD-L1/TGFβ Trap, which includes a first polypeptide that includes the amino acid sequence of SEQ ID NO: 3, and a second polypeptide that includes the amino acid sequence of SEQ ID NO: 1. In certain embodiments, patients with heavily pretreated recurrent or refractory unresectable Stage IV gastric cancer are treated by intravenously administering anti-PD-L1/TGFβ Trap at a dose of about 1200 mg once every 2 weeks for 2-30 weeks. In certain embodiments, the treated patients received at least 3 prior anticancer therapies. In certain embodiments, the treated patients received at least 4 prior anticancer therapies.

In certain embodiments, patients with pretreated colorectal cancer (CRC) are treated by intravenously administering about 1200 mg-about 2400 mg (e.g., about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 2400 mg, about 1400 mg to about 2400 mg, about 1500 mg to about 2400 mg, about 1600 mg to about 2400 mg, about 1700 mg to about 2400 mg, about 1800 mg to about 2400 mg, about 1900 mg to about 2400 mg, about 2000 mg to about 2400 mg, about 2100 mg to about 2400 mg, about 2200 mg to about 2400 mg, or about 2300 mg to about 2400 mg) of anti-PD-L1/TGFβ Trap, which includes a first polypeptide that includes the amino acid sequence of SEQ ID NO: 3, and a second polypeptide that includes the amino acid sequence of SEQ ID NO: 1. In certain embodiments, patients with pretreated colorectal cancer (CRC) are treated with anti-PD-L1/TGFβ Trap at a dose of about 1200 mg once every 2 weeks for 2-38 weeks. In certain embodiments, the treated patients received at least 3 prior anticancer therapies.

### Delivery Device

In one aspect, the present disclosure provides a drug delivery device including a formulation comprising about 500 mg-about 3000 mg of a protein including a first polypeptide and a second polypeptide, the first polypeptide includes: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ), the second polypeptide includes at least a variable region of a light chain of an antibody that binds PD-L1, and the heavy chain of the first polypeptide and the light chain of the second polypeptide, when combined, form an antigen binding site that binds PD-L1.

In certain embodiments, the device may be a bag, a pen, or a syringe. In certain embodiments, the bag may be connected to a channel comprising a tube and/or a needle.

In certain embodiments of the present disclosure, the drug delivery device may include about 500 mg to about 3000 mg (e.g., about 500 mg to about 3000 mg, about 500 mg to about 2900 mg, about 500 mg to about 2800 mg, about 500 mg to about 2700 mg, about 500 mg to about 2600 mg, about 500 mg to about 2500 mg, about 500 mg to about 2400 mg, about 500 mg to about 2300 mg, about 500 mg to about 2200 mg, about 500 mg to about 2100 mg, about 500 mg to about 2000 mg, about 500 mg to about 1900 mg, about 500 mg to about 1800 mg, about 500 mg to about 1700 mg, about 500 mg to about 1600 mg, about 500 mg to about 1500 mg, about 500 mg to about 1400 mg, about 500 mg to about 1300 mg, about 500 mg to about 1200 mg, about 500 mg to about 1100 mg, about 500 mg to about 1000 mg, about 500 mg to about 900 mg, about 500 mg to about 800 mg, about 500 mg to about 700 mg, about 500 mg to about 600 mg, about 600 mg to about 3000 mg, about 700 mg to about 3000 mg, about 800 mg to about 3000 mg, about 900 mg to about 3000 mg, about 1000 mg to about 3000 mg, about 1100 mg to about 3000 mg, about 1200 mg to about 3000 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, or about 2900 mg to about 3000 mg) of a protein of the present disclosure (e.g., anti-PD-L1/TGFβ Trap, which includes a first polypeptide that includes the amino acid sequence of SEQ ID NO: 3, and a second polypeptide that includes the amino acid sequence of SEQ ID NO: 1). In certain embodiments, the drug delivery device may include about 500 to about 1200 mg dose of a protein of the present disclosure (e.g., anti-PD-L1/TGFβ Trap, which includes a first polypeptide that includes the amino acid sequence of SEQ ID NO: 3, and a second polypeptide that includes the amino acid sequence of SEQ ID NO: 1). In certain embodiments, the drug delivery device may include about 500 mg dose of the protein of the present disclosure (e.g., anti-PD-L1/TGFβ Trap, which includes a first polypeptide that includes the amino acid sequence of SEQ ID NO: 3, and a second polypeptide that includes the amino acid sequence of SEQ ID NO: 1). In certain embodiments, the drug delivery device includes about 1200 mg dose of a protein of the present disclosure (e.g., anti-PD-L1/TGFβ Trap, which includes a first polypeptide that includes the amino acid sequence of SEQ ID NO: 3, and a second polypeptide that includes the amino acid sequence of SEQ ID NO: 1). In certain embodiments, the drug delivery device includes about 1200 mg or about 1800 mg dose of the protein product with a first polypeptide that includes the amino acid sequence of SEQ ID NO: 3, and the second polypeptide that includes the amino acid sequence of SEQ ID NO: 1.

In certain embodiments, the drug delivery device includes about 1200 mg dose of the protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)). In certain embodiments, the drug delivery device may include about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2100 mg, about 2200 mg, about 2300 mg, or about 2400 mg of the protein of the present disclosure (e.g., anti-PD-L1/TGFβ Trap).

### Kit

In one aspect, the present disclosure provides a kit including one or more vessels collectively including a formulation of about 500 mg to about 2400 mg (e.g., about 500 mg to about 2400 mg, about 500 mg to about 2300 mg, about 500 mg to about 2200 mg, about 500 mg to about 2100 mg, about 500 mg to about 2000 mg, about 500 mg to about 1900 mg, about 500 mg to about 1800 mg, about 500 mg to about 1700 mg, about 500 mg to about 1600 mg, about 500 mg to about 1500 mg, about 500 mg to about 1400 mg, about 500 mg to about 1300 mg, about 500 mg to about 1200 mg, about 500 mg to about 1100 mg, about 500 mg to about 1000 mg, about 500 mg to about 900 mg, about 500 mg to about 800 mg, about 500 mg to about 700 mg, about 500 mg to about 600 mg, about 600 mg to about 2400 mg, about 700 mg to about 2400 mg, about 800 mg to about 2400 mg, about 900 mg to about 2400 mg, about 1000 mg to about 2400 mg, about 1100 mg to about 2400 mg, about 1200 mg to about 2400 mg, about 1300 mg to about 2400 mg, about 1400 mg to about 2400 mg, about 1500 mg to about 2400 mg, about 1600 mg to about 2400 mg, about 1700 mg to about 2400 mg, about 1800 mg to about 2400 mg, about 1900 mg to about 2400 mg, about 2000 mg to about 2400 mg, about 2100 mg to about 2400 mg, about 2200 mg to about 2400 mg, or about 2300 mg to about 2400 mg) of a protein including a first polypeptide and a second polypeptide, the first polypeptide includes: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ), the second polypeptide includes at least a variable region of a light chain of an antibody that binds PD-L1, and the heavy chain of the first polypeptide and the light chain of the second polypeptide, when combined, form an antigen binding site that binds PD-L1.

In certain embodiments of the present disclosure, the vessels collectively may include a dose of about 500 mg to about 2400 mg (e.g., about 500 mg to about 2400 mg, about 500 mg to about 2300 mg, about 500 mg to about 2200 mg, about 500 mg to about 2100 mg, about 500 mg to about 2000 mg, about 500 mg to about 1900 mg, about 500 mg to about 1800 mg, about 500 mg to about 1700 mg, about 500 mg to about 1600 mg, about 500 mg to about 1500 mg, about 500 mg to about 1400 mg, about 500 mg to about 1300 mg, about 500 mg to about 1200 mg, about 500 mg to about 1100 mg, about 500 mg to about 1000 mg, about 500 mg to about 900 mg, about 500 mg to about 800 mg, about 500 mg to about 700 mg, about 500 mg to about 600 mg, about 600 mg to about 2400 mg, about 700 mg to about 2400 mg, about 800 mg to about 2400 mg, about 900 mg to about 2400 mg, about 1000 mg to about 2400 mg, about 1100 mg to about 2400 mg, about 1200 mg to about 2400 mg, about 1300 mg to about 2400 mg, about 1400 mg to about 2400 mg, about 1500 mg to about 2400 mg, about 1600 mg to about 2400 mg, about 1700 mg to about 2400 mg, about 1800 mg to about 2400 mg, about 1900 mg to about 2400 mg, about 2000 mg to about 2400 mg, about 2100 mg to about 2400 mg, about 2200 mg to about 2400 mg, or about 2300 mg to about 2400 mg) of the protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)). In certain embodiments, the vessels collectively may include 500 to 1800 mg dose of the protein of the present disclosure (e.g., anti-PD-L1/TGFβ Trap). In certain embodiments, the vessels collectively may include a 500 mg dose of the protein of the present disclosure (e.g., anti-PD-L1/TGFβ Trap). In certain embodiments, the vessels collectively may include a 1200 mg dose of the protein of the present disclosure (e.g., anti-PD-L1/TGFβ Trap). In certain embodiments, the vessels collectively may include an 1800 mg dose of the protein of the present disclosure (e.g., anti-PD-L1/TGFβ Trap). In certain embodiments, the formulation is prepared and packaged as a liquid formulation and stored as about 250 mg/vial to about 1000 mg/vial (e.g., about 250 mg/vial to about 1000 mg/vial, about 250 mg/vial to about 900 mg/vial, about 250 mg/vial to about 800 mg/vial, about 250 mg/vial to about 700 mg/vial, about 250 mg/vial to about 600 mg/vial, about 250 mg/vial to about 500 mg/vial, about 250 mg/vial to about 400 mg/vial, about 250 mg/vial to about 300 mg/vial, about 300 mg/vial to about 1000 mg/vial, about 400 mg/vial to about 1000 mg/vial, about 500 mg/vial to about 1000 mg/vial, about 600 mg/vial to about 1000 mg/vial, about 700 mg/vial to about 1000 mg/vial, about 800 mg/vial to about 1000 mg/vial, or about 900 mg/vial to about 1000 mg/vial). For example, in certain embodiments, the formulation is a liquid formulation and stored as about 600 mg/vial, or stored as about 250 mg/vial.

In certain embodiments, the vessels collectively may include a dose of about 1200 mg or about 1800 mg of the protein product with a first polypeptide that includes the amino acid sequence of SEQ ID NO: 3, and the second polypeptide that includes the amino acid sequence of SEQ ID NO: 1. In certain embodiments, the formulation is prepared and packaged as a liquid formulation and stored as about 250 mg/vial to about 1200 mg/vial (e.g., about 250 mg/vial to about 1200 mg/vial, about 250 mg/vial to about 1100 mg/vial, about 250 mg/vial to about 1000 mg/vial, about 250 mg/vial to about 900 mg/vial, about 250 mg/vial to about 800 mg/vial, about 250 mg/vial to about 700 mg/vial, about 250 mg/vial to about 600 mg/vial, about 250 mg/vial to about 500 mg/vial, about 250 mg/vial to about 400 mg/vial, about 250 mg/vial to about 300 mg/vial, about 300 mg/vial to about 1200 mg/vial, about 400 mg/vial to about 1200 mg/vial, about 500 mg/vial to about 1200 mg/vial, about 600 mg/vial to about 1200 mg/vial, about 700 mg/vial to about 1200 mg/vial, about 800 mg/vial to about 1200 mg/vial, about 900 mg/vial to about 1200 mg/vial, about 1000 mg/vial to about 1200 mg/vial, or about 1100 mg/vial to about 1200 mg/vial) of the protein product with a first polypeptide that includes the amino acid sequence of SEQ ID NO: 3, and the second polypeptide that includes the amino acid sequence of SEQ ID NO: 1. For example, in certain embodiments, the formulation is a liquid formulation and stored as about as about 1200 mg/vial, or stored as about 600 mg/vial, or stored as about 250 mg/vial.

In certain embodiments, the vessels collectively may include about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2100 mg, about 2200 mg, about 2300 mg, or about 2400 mg of the protein of the present disclosure (e.g., anti-PD-L1/TGFβ Trap (e.g., including a first polypeptide that includes the amino acid sequence of SEQ ID NO: 3, and a second polypeptide that includes the amino acid sequence of SEQ ID NO: 1)).

In certain embodiments, the formulation in the vessels may be a lyophilized formulation or a liquid formulation.

In certain embodiments, the formulation may be packed in kits containing a suitable number of vials. The information on the medication may be included, which are in accordance with approved submission documents. The kit may be shipped in transport cool containers (2° C. to 8° C.) that are monitored with temperature control devices.

The formulation may be stored at 2° C. to 8° C. until use. In certain embodiments, the freeze-dried drug product may be reconstituted with 4.5 mL of water for Injection and diluted with about 0.9% saline solution (sodium chloride injection) while the liquid formulation may be diluted with about 0.9% saline solution. The vials of the formulations may be sterile and nonpyrogenic, and may not contain bacteriostatic preservatives.

In certain embodiments, the delivery device is an injector pen. An injector pen is a device designed to allow a user to self-administer a pre-measured dose of a medicament composition subcutaneously or intramuscularly. An injector pen may have a housing, inside of which is a cartridge. The cartridge may have one or several chambers containing medicament compositions or components thereof and is adapted to be attached to a needle assembly. The cartridge can hold either a pre-mixed liquid medicament or a solid medicament and a liquid that are mixed prior to injection. The housing may carry an actuation assembly with a stored energy source, for example, a compressed spring. Activation of the actuation assembly causes a sequence of movements, whereby the needle extends from the injector pen into the user so that the medicament compound is then forced through the needle and into the user. After delivery of the dose of medicament into the injection site, the needle may remain in an extended position. If the injector pen is of the type designed to carry plural components of the medicament composition in separate, sealed compartments, structure may be included that forces the components to mix when the actuation assembly is activated.

### Protein Production

The antibody-cytokine Trap proteins are generally produced recombinantly, using mammalian cells containing a nucleic acid engineered to express the protein. Although one example of a suitable cell line and protein production method is described in Examples 1 and 2 of US 20150225483 A1, a wide variety of suitable vectors, cell lines and protein production methods have been used to produce antibody-based biopharmaceuticals and could be used in the synthesis of these antibody-cytokine Trap proteins.

### Therapeutic Indications

The anti-PD-L1/TGFβ Trap proteins described in the application (e.g., including a first polypeptide that includes the amino acid sequence of SEQ ID NO: 3, and a second polypeptide that includes the amino acid sequence of SEQ ID NO: 1) can be used to treat cancer or reduce tumor growth in a patient. Exemplary cancers include non-small cell lung cancer, melanoma, pancreatic cancer, colorectal cancer (e.g., pretreated colorectal cancer (CRC)), ovarian cancer, glioblastoma, gastric cancer (e.g., pretreated recurrent or refractory unresectable Stage IV gastric cancer), biliary tract cancer, esophageal cancer (squamous cell carcinoma or adenocarcinoma), adenoma of the head or the neck, and squamous carcinoma of the head or the neck.

The cancer or tumor to be treated with an anti-PD-L1/TGFβ Trap may be selected based on the expression or elevated expression of PD-L1 and TGFβ in the tumor, the correlation of their expression levels with prognosis or disease progression, and preclinical and clinical experience on the sensitivity of the tumor to treatments targeting PD-L1 and TGFβ. Such cancers or tumors include but are not limited to colorectal, breast, ovarian, pancreatic, gastric, prostate, renal, cervical, bladder, head and neck, liver, non-small cell lung cancer, advanced non-small cell lung cancer, melanoma, Merkel cell carcinoma, and mesothelioma.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: Packaging of Intravenous Drug Formulation

The formulation of anti-PD-L1/TGFβ Trap is prepared as a lyophilized formulation or a liquid formulation. For preparing the lyophilized formulation, 45 mg of freeze-dried anti-PD-L1/TGFβ Trap is sterilized and stored in one container. Several such containers are then packaged in a kit for delivering a specific body weight independent dose to a subject diagnosed with a cancer or a tumor. Depending on the dose requirement, the kit contains 12-60 vials. Alternatively, the formulation is prepared and packaged as a liquid formulation and stored as 250 mg/vial to 1000 mg/vial. For example, the formulation is a liquid formulation and stored as 600 mg/vial, or stored as 250 mg/vial.

The formulation is used for treating cancer or tumor, for example, non-small cell lung cancer, melanoma, pancreatic cancer, colorectal cancer (e.g., pretreated colorectal cancer (CRC)), ovarian cancer, glioblastoma, gastric cancer (e.g., pretreated recurrent or refractory unresectable Stage IV gastric cancer), biliary tract cancer, esophageal cancer (squamous cell carcinoma or adenocarcinoma), adenoma of the head or the neck, and squamous carcinoma of the head or the neck.

A subject diagnosed with such a cancer or tumor is intravenously administered a formulation containing 500 mg to 2000 mg of anti-PD-L1/TGFβ Trap. For example, the subject is intravenously administered 500 mg of anti-PD-L1/TGFβ Trap or 1200 mg of anti-PD-L1/TGFβ Trap. The intravenous administration is from a saline bag, and administration is once in two weeks. The amount of the anti-PD-L1/TGFβ Trap administered to a subject is independent of the subject's body weight.

### Example 2: BW-Independent Dosing Regimen

In one exemplary embodiment, the BW-independent dose of 500 mg or 1200 mg was administered to subjects with non-small cell lung cancer (NSCLC) once every two weeks. The administration was performed intravenously for about an hour (−10 minutes/+20 minutes, i.e., 50 minutes to 80 minutes). In order to mitigate potential infusion-related reactions, premedication with an antihistamine and with paracetamol (acetaminophen) (for example, 25-50 mg diphenhydramine and 500-650 mg paracetamol [acetaminophen] IV or oral equivalent) approximately 30 to 60 minutes prior to each dose of was administered for the first 2 infusions. If Grade ≥2 infusion reactions were seen during the first two infusions premedication was not stopped. Steroids as premedication were not permitted.

For subjects who achieved a PR or CR on anti-PD-L1/TGFβ Trap therapy and then subsequently developed disease progression after stopping therapy, 1 re-initiation course of treatment at the same dose and schedule and treatment duration up to 12 months was allowed. In this example, subjects were ≥18 years and had histologically or cytologically proven metastatic or locally advanced solid tumors, for which no effective standard therapy exists or standard therapy had failed. Preferred subjects had adequate hematological function defined by white blood cell (WBC) count ≥3×109/L with absolute neutrophil count (ANC)≥1.5×109/L, lymphocyte count ≥0.5×109/L, platelet count ≥120×109/L, and Hgb≥9 g/dL (in absence of blood transfusion); adequate hepatic function defined by a total bilirubin level ≤1.5×ULN, an AST level ≤2.5×ULN, and an ALT level ≤2.5×ULN; and adequate renal function defined by an estimated creatinine clearance >50 mL/min according to the Cockcroft-Gault formula or by measure of creatinine clearance from 24 hour urine collection. For subjects with liver involvement in their tumor, AST≤5.0×ULN, ALT≤5.0×ULN, and bilirubin ≤3.0 was acceptable.

In other embodiments, certain subjects had:
Histologically or cytologically confirmed Stage Mb or IV NSCLC with relapsed, refractory or progressive disease on or after a single line of platinum-based chemotherapy, and no previous treatment with combination immunotherapy.
Histologically confirmed hepatocellular carcinoma that was unresectable or advanced disease not amenable to curative resection, with cancer that progressed following 1 line of prior sorafenib therapy (at least 14 days of sorafenib at least 400 mg per day) or that was previously considered to be sorafenib intolerant.
Histologically confirmed Stage IV, or recurrent NSCLC with no prior systemic anticancer therapy.
Histologically confirmed Stage IV, or recurrent NSCLC in patients who received and failed platinum-based chemotherapy as monotherapy and failed with disease progression.
Histologically confirmed Stage IV, or recurrent NSCLC in patients who received and failed platinum-based chemotherapy as monotherapy and failed with disease progression and have received anti-PD-1 or anti-PD-L1 as monotherapy and failed with disease progression.
Unresectable Stage III or metastatic (Stage IV) melanoma.
Histologically confirmed pancreatic adenocarcinoma that was unresectable, advanced, and/or metastatic, without previous radiotherapy.
Histologically confirmed adenocarcinoma of the colon or rectum that progressed during or after a second-line of systemic treatment including a fluoropyrimidine, oxaliplatin, irinotecan and/or bevacizumab.
Triple-negative breast cancer that progressed during or after first-line of chemotherapy.
Histologically confirmed epithelial ovarian, fallopian tube, or peritoneal cancer with unresectable metastatic disease that is platinum resistant/refractory in patients previously treated with at least 2 chemotherapy regimens including platinum and taxane agents.
Histologically confirmed recurrent or metastatic esophageal adenocarcinoma, with unresectable (Stage III or IV) disease in patients who have received at least one previous platinum-containing chemotherapy regimen.
Histologically confirmed Grade IV malignant glioma previously treated with radiotherapy and temozolomide.
Histologically confirmed recurrent or metastatic Squamous Cell Carcinoma Head and Neck (SCCHN) (oral cavity, pharynx, larynx), Stage III/IV, with tumor progression or recurrence within 6 months of last dose of platinum therapy in the adjuvant (i.e. with radiation after surgery), primary (i.e., with radiation), recurrent, or metastatic setting.
Histologically confirmed recurrent or persistent squamous cell carcinoma, adenosquamous carcinoma, or adenocarcinoma of the cervix following standard of care treatment with systemic therapy for advanced disease.
Histologically or cytologically confirmed recurrent or refractory unresectable Stage IV gastric or gastro-esophageal junctional adenocarcinoma for which no standard therapy exists or standard therapy has failed.
Histologically or cytologically confirmed esophageal squamous cell cancer for which no standard therapy exists or standard therapy has failed.
Histologically or cytologically confirmed biliary tract cancer who have failed or are intolerant to one line of systemic treatment.

Selected subjects did not have active tuberculosis or an autoimmune disease that might deteriorate when receiving an immunostimulatory agent.

### Example 3: Efficacy Assessments

Tumor response assessment is performed by CT scan or MRI. Scans performed at baseline are repeated at subsequent visits. In general, lesions detected at baseline are followed using the same imaging methodology and preferably the same imaging equipment at subsequent tumor evaluation visits. Skin metastasis can be used as target lesions according to RECIST 1.1 using measurements by caliper, if they fulfill RECIST 1.1 for target lesions.

### Example 4: Treatment of Advanced NSCLC Patients Refractory or Resistant to Prior Treatment with Anti-PD-1 or Anti-PD-L1 Agent

Objective: Histologically confirmed Stage IV, or recurrent NSCLC in patients who received and failed platinum-based chemotherapy as monotherapy and failed with disease progression and have received anti-PD-1 or anti-PD-L1 as monotherapy and failed with disease progression, were selected for treatment with 1200 mg of anti-PD-L1/TGFβ Trap therapy. The rationale for using anti-PD-L1/TGFβ Trap in these NSCLC PDx-fail sub-cohorts was that simultaneous neutralization of TGF-β, a molecule known to inhibit tumor immune activation, might stimulate clinical response in patients who failed to respond to PDx therapy alone.

Summary: Patients who had advanced NSCLC and were previously treated with anti-PD-1 or anti-PD-L1 agent (“PDx therapy”) and subsequently had documented disease progression, were selected for intravenous administration of 1200 mg of anti-PD-L1/TGFβ Trap. Patient best overall response (BOR) to prior PDx therapy was documented. A sub-cohort of patients with progressive disease (PD) following prior PDx therapy was considered as “primary refractory”, i.e., among these patients disease progression was observed following PDx therapy initiation without any observed benefit from the treatment. Another sub-cohort of the patients was characterized as “acquired resistant”, i.e., the patients' disease initially responded to prior PDx therapy, but the patients ultimately reverted to disease progression stage. The acquired resistant patients were characterized with BOR of stable disease (SD), partial response (PR) or complete response (CR) to prior PDx therapy before the subsequent disease progression.

Study Design and Results: A total of 83 patients were treated with anti-PD-L1/TGFβ Trap at a dose of 1200 mg every 2 weeks. The median follow-up was 27.3 weeks. Primary endpoint is BOR per RECIST v1.1, secondary endpoints being safety/tolerability. Baseline characteristics of patients are listed in the table below. In summary, this was a heavily pre-treated patient population with 74.7% of patients receiving greater than 3 prior treatment regimens, and included a range of ages and genders. Response to prior PDx therapy was approximately balanced (primary refractory, 43.4%; acquired resistant, 53%). Additionally, all patients had a biopsy within 28 days of treatment start and a majority (65.1%) were noted to have ≥1% tumor cell PD-L1 expression based upon the Dako 73-10 PD-L1 assay.

TABLE 6
Patient characteristics

| Characteristics, n (%) | N = 83 | |
|---|---|---|
| Sex | | |
| Male | 56 | (67.5) |
| Female | 27 | (32.5) |
| Age | | |
| <65 | 46 | (55.4) |
| ≥65 | 36 | (43.4) |
| ECOG performance status | | |
| 0 | 27 | (32.5) |
| 1 | 54 | (65.1) |
| 2 | 1 | (1.2) |
| ≥3 | 0 | (0.0) |
| Missing | 1 | (1.2) |
| Tumor PD-L1 expression | | |
| ≥1% | 54 | (65.1) |
| <1% | 21 | (25.3) |
| Unknown | 8 | (9.6) |
| Number of prior anticancer drug therapies | | |
| 0 | 0 | (0.0) |
| 1 | 0 | (0.0) |
| 2 | 21 | (25.3) |
| 3 | 26 | (31.3) |
| ≥4 | 36 | (43.4) |
| Response to prior anti-PD-1/PD-L1 therapy | | |
| Primary Refractory | 36 | (43.4) |
| Acquired Resistance | 44 | (53) |
| Missing | 3 | (3.6) |

As of data cut-off date at the time of analysis, a total of 2 patients (2.4%) had a confirmed response by investigator per RECIST v1.1. Disease control was reported in 20 patients (24.1%). By independent radiologic review, 3 patients (3.6%) had a confirmed partial response. Moreover, an additional 1 confirmed PR and 1 unconfirmed PR were reported by investigators, therefore increasing the unconfirmed ORR to 4.8%; 6 patients were continued to be treated. Responses occurred in patients with primary refractory disease and those with acquired resistance to prior PDx therapy.

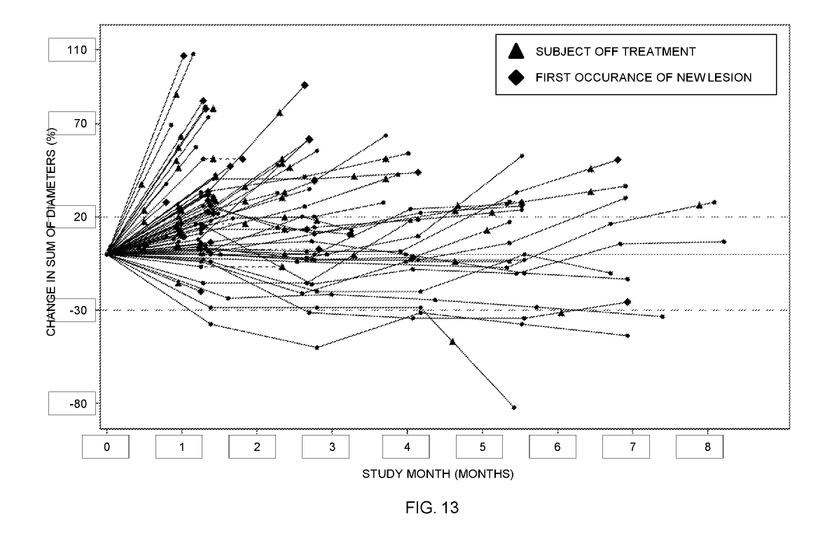

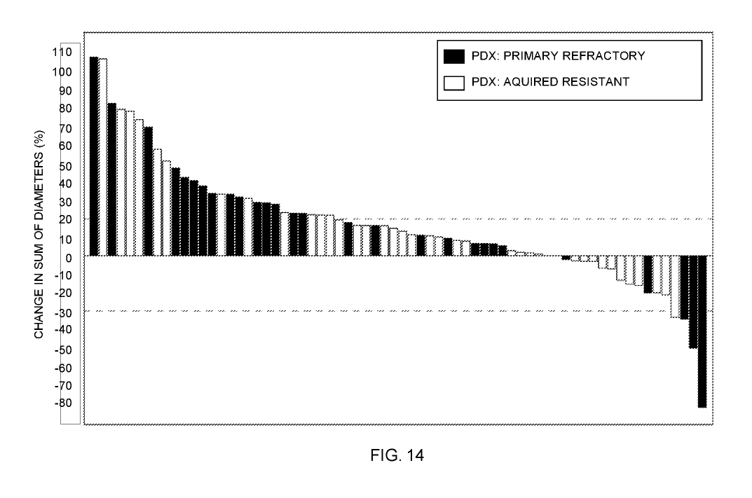

As shown in FIG. 13, patients with previously progressive disease (both with primary refractory and acquired resistant disease) achieved significant disease stabilization. Patients with disease response and disease stabilization were noted to have a range in prior treatments prior to initiating this study, and even had a range of treatments immediately prior to starting on trial, suggesting clinical activity of anti-PD-L1/TGFβ Trap in a heterogeneous population of patients with prior PDx exposure. Responses and disease control were noted in both high and low PD-L1 expressing patients irrespective of PD-L1 status at trial start and also in patients with high or low circulating TGF-β1 plasma levels.

In NSCLC PDx-fail patients, anti-PD-L1/TGFβ Trap was overall well tolerated by patients with treatment-related adverse event (TRAE) rates similar to that seen with other anti-PD-1 or anti-PD-L1 monotherapies. Most patients (n=60, 72.3%) experienced any TRAE, with a smaller proportion (n=19, 22.9%) experiencing a grade 3 or higher event. The table representing TRAEs in ≥5% of patients is reported below. Fatigue/asthenia was most common (36.1%, G3+ 6.0%), followed by pruritis (21.7%, G3+ 2.4%) and decreased appetite (16.9%, G3+1.2%). One patient discontinued the study due to a treatment-related AE (G2, plaque eczema). One patient died from pneumonia assessed by the investigator as treatment related. Of note, cutaneous lesions occurred in 5 patients (6.0%), including keratoacanthoma and squamous cell carcinoma (similar to other TGF-β-inhibiting agents) and were well managed by surgical excision.

TABLE 7
Treatment-related adverse events (TRAEs)

| N = 85 | Any Grade | Grade ≥3 |
|---|---|---|
| Any TRAE, n (%) | 60 (72.3) | 19 (22.9) |
| Anemia | 5 (6.0) | 1 (1.2) |
| Arthralgia | 6 (7.2) | 1 (1.2) |
| Decreased appetite | 14 (16.9) | 1 (1.2) |
| Diarrhea | 6 (7.2) | 0 (0.0) |
| Dry skin | 5 (6.0) | 0 (0.0) |
| Epistaxis | 8 (9.6) | 0 (0.0) |
| Fatigue/Asthenia | 30 (36.1) | 5 (6.0) |
| Pruritus | 18 (21.7) | 2 (2.4) |
| Rash maculopapular | 6 (7.2) | 1 (1.2) |

In summary, anti-PD-L1/TGFβ Trap was found to be an innovative first-in-class bifunctional fusion protein designed to simultaneously target 2 immune suppressive pathways: PD-L1 and TGF-0. Inhibition of the TGF-β pathway, thereforeaids in overcoming treatment failure to anti-PD-1/PD-L1 agents. Treatment with anti-PD-L1/TGFβ Trap resulted in initial clinical activity in patients with heavily pre-treated NSCLC with disease primary refractory or acquired resistant to prior treatment with anti-PD-1 or anti-PD-L1 therapy.

### Example 5: Treatment of Pretreated Recurrent or Refractory Stage IV Gastric Cancer Patients

Objective: Patients with heavily pretreated recurrent or refractory unresectable Stage IV gastric cancer were selected for treatment with 1200 mg of anti-PD-L1/TGFβ Trap therapy and safety and efficacy was assessed.

Study Design and Results: A total of 31 patients were treated with anti-PD-L1/TGFβ Trap at a dose of 1200 mg every 2 weeks until confirmed progressive disease, unacceptable toxicity, or trial withdrawal. The cohort consisted of a heavily pretreated Asian patient population with 67.7% receiving at least 3 prior anticancer therapies and 29.3% at least 4 prior anticancer therapies.

Baseline characteristics of patients are listed in the table 8 below.

TABLE 8
Patient characteristics

| Characteristic | N = 31 |
|---|---|
| Sex, n (%) | |
| Male | 26 (83.9) |
| Female | 5 (16.1) |
| Age, years | |
| Median | 64 |
| Range | 45-82 |
| Number of prior anticancer therapies, n (%) | |
| 1 | 4 (12.9) |
| 2 | 6 (19.4) |
| 3 | 12 (38.7) |
| ≥4 | 9 (29.3) |
| ECOG performance status, n (%) | |
| 0 | 8 (25.8) |
| 1 | 23 (74.2) |

As of data cut-off date at the time of analysis, the patients had received anti-PD-L1/TGFβ Trap for a median duration of 6.1 (range: 2-30) weeks. Among the 31 evaluable patients, 5 patients had a confirmed partial response and 5 patients had stable disease (SD) as their BOR per RECIST v1.1 as assessed by the investigator. The overall response rate (ORR) was 16.1% and the disease control rate (DCR) was 32.3%.

TABLE 9
Patient characteristics

| | N = 31 |
|---|---|
| BOR, n (%) | |
| CR | 0 |
| PR | 5 (16.1) |
| SD | 5 (16.1) |
| PD | 16 (51.6) |
| NE | 5 (16.1) |
| ORR, n (%) | |
| Response rate (CR + PR) | 5 (16.1) |
| 95% CI | 5.5-33.7 |
| DCR, n (%) | |
| Response rate (CR + PR + SD) | 10 (32.3) |
| 95% CI | 16.7-51.4 |

Anti-PD-L1/TGFβ Trap was overall well tolerated by patients with treatment-related adverse event (TRAE) rates similar to that seen with other anti-PD-1/PD-L1 monotherapies. 14 patients (45.2%) experienced treatment-related adverse events. 4 patients (12.9%) experienced grade 3 TRAEs. No treatment-related grade 4 AEs occurred. 1 grade 5 event (total 5 doses received) was considered possibly related to treatment, but suspected rupture of preexisting thoracic aortic aneurysm was cited as other probable cause by the investigator.

TABLE 10
Treatment-related adverse events (TRAEs)

| N = 31 | Any grade | Grade ≥3 |
|---|---|---|
| Any TRAE, n (%) | 14 (45.2) | 5 (16.1) |
| Rash maculopapular | 6 (19.4) | 1 (3.2) |
| Anemia | 3 (9.7) | 2 (6.5) |
| Rash | 3 (9.7) | 1 (3.2) |
| Diarrhea | 2 (6.5) | 1 (3.2) |
| Fatigue | 2 (6.5) | 0 |
| Infusion-related reaction | 2 (6.5) | 0 |
| Pruritus | 2 (6.5) | 0 |
| (sudden death) | 1 (3.2) | 1 (3.2) |

In summary, anti-PD-L1/TGFβ Trap was found to be an innovative first-in-class bifunctional fusion protein designed to simultaneously target 2 immune suppressive pathways: PD-L1 and TGF-β. Inhibition of the TGF-β pathway may aid in overcoming treatment failure to anti-PD-1/PD-L1 agents. Treatment with anti-PD-L1/TGFβ Trap resulted in initial clinical activity in Asian patients with heavily pretreated gastric cancer.

### Example 6: Treatment of Patients with Heavily Pretreated Colorectal Cancer (CRC)

Background and Objective: CRC is respectively the second and third most common cancer in women and men worldwide. Recently, 4 consensus molecular subgroups (CMS) of CRC have been described—including the poor-prognosis, mesenchymal CMS4 group which is characterized by angiogenic, inflammatory, and immunosuppressive qualities. It is hypothesized that TGF-β may play a role in mediating this immuno-suppressive phenotype providing a rationale for using anti-PD-L1/TGFβ Trap in these patients. Anti-PD-L1 therapy has shown substantial activity for patients with defective mismatch repair (e.g. microsatellite instability-high (MSI-H)) CRC, however only about 4% of patients with metastatic CRC have MSI-H tumors, and these treatments have had minimal activity in patients with proficient mismatch repair.

Sudy Design and Results: A total of 32 patients were treated with anti-PD-L1/TGFβ Trap at a dose of 1200 mg every 2 weeks. The median duration of treatment was 7.1 weeks (range: 2-38), and as of the data cut-off date at the time of analysis, 2 patients remained on active treatment. Primary endpoint is BOR per RECIST v1.1, secondary endpoints being safety/tolerability. Baseline characteristics of patients are listed in the table below. In summary, this was a heavily pre-treated patient population with 87.5% of patients receiving greater than 3 prior treatment regimens, overall good clinical status (PS 0-1) and included a range of ages and genders. Approximate 34% of tumors were KRAS-mutated, and a majority of patients (81.3%) had <1% PD-L1 expression on tumor cells based upon the Dako 73-10 PD-L1 assay. Tumor-sidedness was not prospectively collected in the database, but was determined based upon the patient's prior cancer surgery history. Using this clinical assessment, 40.6% of tumors were noted to be left sided, 28.1% were right-sided, and 31.3% were unable to be determined based upon available data.

TABLE 11
Patient characteristics

| Characteristic | N = 32 |
|---|---|
| Sex, n (%) | |
| Male | 16 (50.0) |
| Female | 16 (50.0) |
| Age, years | |
| Median | 60 |
| Range | 26-81 |
| Number of prior anticancer therapies, n (%) | |
| 0 | 0 |
| 1 | 0 |
| 2 | 4 (12.5) |
| 3 | 9 (28.1) |
| ≥4 | 19 (59.4) |
| ECOG performance status, n (%) | |
| 0 | 11 (34.4) |
| 1 | 21 (65.6) |
| KRAS mutational status, n (%) | |
| Wild type | 11 (34.4) |
| Mutant | 21 (65.6) |
| PD-L1 expression in tumor cells, n (%) | |
| ≥1% | 3 (9.4) |
| <1% | 26 (81.3) |
| Non evaluable | 3 (9.4) |
| Primary tumor type, n (%) | |
| Adenocarcinoma of the colon | 23 (71.9) |
| Adenocarcinoma of the rectum | 9 (28.1) |
| Tumor sidedness, n (%) | |
| Left | 13 (40.6) |
| Right | 9 (28.1) |
| Non evaluable | 10 (31.3) |

As of data cut-off date at the time of analysis, 1 patient (3.1%) had a confirmed partial response (PR). One patient additional had stable disease, and 27 patients had progressive disease as best overall response. Response criteria was adjudicated by independent review committee, and was defined per RECISTv1.1. The patient with a PR had CRC that was proficient in mismatch repair (i.e. microsatellite stable), CMS4, KRAS mutant, and PD-L1+ (PD-L1>1% in tumor cells by IHC). This patient had the highest tumor cell PD-L1 expression in our cohort (20%). As of Nov. 1, 2017, the patient with a PR remained on-study (12.5 months) with ongoing partial response. An additional patient remains on treatment at 13 months with unassessable disease due to receipt of palliative RT to a target lesion during month 5 of treatment. No new lesions have occurred since that time, and non-target lesions remain stable. This patient's CRC was KRAS-mutated; CMS and microsatellite status are pending, PD-L1 status is unknown.

In CRC patients, anti-PD-L1/TGFβ Trap was overall well tolerated by patients with treatment-related adverse event (TRAE) rates similar to that seen with other anti-PD-1/PD-L1 monotherapies. Most patients (n=22, 68.8%) experienced any TRAE, with a smaller proportion (n=4, 12.5%) experiencing a grade 3 event. There were no grade 4/5 TRAEs. The table representing TRAEs in ≥5% of patients is reported below. Anemia, diarrhea, infusion-related reaction and nausea were most common (all n=5, 15.6%). There was a single grade 3 related anemia event (3.1%). Only one TRAE led to treatment discontinuation (grade 3 enteritis). The event occurred concurrently with disease progression.

TABLE 12
Treatment-related adverse events (TRAEs)

| N = 32 | Any Grade | Grade 3 |
|---|---|---|
| Any TRAE, n (%) | 22 (68.8) | 4 (12.5) |
| Anemia | 5 (15.6) | 1 (3.1) |
| Diarrhea | 5 (15.6) | 0 |
| Infusion-related reaction | 5 (15.6) | 0 |
| Nausea | 5 (15.6) | 0 |
| Decreased appetite | 4 (12.5) | 0 |
| Fatigue | 3 (9.4) | 1 (3.1) |
| Myalgia | 3 (9.4) | 0 |
| Pyrexia | 3 (9.4) | 0 |
| Vomiting | 3 (9.4) | 0 |
| Abdominal pain | 2 (6.3) | 0 |
| Dermatitis acneiform | 2 (6.3) | 0 |
| Malaise | 2 (6.3) | 0 |
| Rash | 2 (6.3) | 0 |
| Rash maculopapular | 2 (6.3) | 0 |
| Stomatitis | 2 (6.3) | 0 |
| Adrenal insufficiency | 1 (3.1) | 1 (3.1) |
| Blood bilirubin increased | 1 (3.1) | 1 (3.1) |
| Enteritis | 1 (3.1) | 1 (3.1) |

In summary, anti-PD-L1/TGFβ Trap was found to be an innovative first-in-class bifunctional fusion protein designed to simultaneously target 2 immune suppressive pathways, TGF-β and PD-L1. Treatment with anti-PD-L1/TGFβ Trap resulted in initial clinical activity in heavily pretreated patients with advanced CRC; 1 patient had a durable PR; 1 patient had SD; and 27 patients had PD as BOR. The patient with a PR ongoing for 8.3 months had CRC that was MSI, CMS4, KRAS-mutant, and PD-L1+. A second patient remains well without recurrence at 13 months after initial progressive disease.

### Example 7: Establishing Efficacious Dose/Dosing Regimen and Exposure in Humans: Preliminary Dose-Response and Exposure-Response in 2^{nd} Line Non Small Cell Lung Cancer (2L NSCLC) Following Once Every 2 Weeks (q2w) Dosing of Anti-PD-L1/TGFβ Trap

In this study, 80 subjects with advanced/recurrent NSCLC, post-platinum treatment and unselected for PD-L1, were administered either 500 mg or 1200 mg of anti-PD-L1/TGFβ Trap once every 2 weeks (q2w) (n=40 per cohort) until disease progression, unacceptable toxicity, or trial withdrawal. Dose-response and exposure-response of the subjects were assessed. As of data cut-off at the time of analysis, a total of 17 subjects remained on treatment with a median follow-up of 35.2 (range, 1.3-47.3) weeks. The investigator-assessed unconfirmed overall response rate (ORR) was 25.0% (500 mg ORR, 22.5%; 1200 mg ORR, 27.5%), with 9 partial responses (PR) seen at 500 mg, and 1 complete response (CR) and 10 PRs at 1200 mg. Clinical activity was observed across PD-L1 expression levels with ORR of 71.4% at 1200 mg noted in patients with ≥80% PD-L1 tumor expression (7 patient). The most common treatment-related adverse events (TRAEs) were pruritus (18.8%), maculopapular rash (17.5%), decreased appetite (12.5%), and asthenia (11.3%). Grade TRAEs occurred in 20 patients (25%). No treatment-related deaths occurred.

For the exposure-response assessment, the population PK model was used to predict first-cycle exposures based on dosing and covariate information from these 80 patients. Specifically, AUC and C_{trough} after a single dose were predicted for every subject using empirical Bayes estimates of population PK parameters (see Table 2 and 3). Predicted exposure data were combined for 500 mg q2w and 1200 mg q2w cohorts to calculate a response rate for each quartile of predicted exposure, as shown in Table 4 and Table 5.

SEQUENCES

SEQ ID NO: 1
Peptide sequence of the secreted anti-PD-L1 lambda light chain
QSALTQPASVSGSPGQSITISCTGTSSDVGGYNYVSWYQQHPGKAPKLMIYDVSNRPS
GVSNRFSGSKSGNTASLTISGLQAEDEADYYCSSYTSSSTRVFGTGTKVTVLGQPKANP
TVTLFPPSSEELQANKATLVCLISDFYPGAVTVAWKADGSPVKAGVETTKPSKQSNNK
YAASSYLSLTPEQWKSHRSYSCQVTHEGSTVEKTVAPTECS
SEQ ID NO: 2
Peptide sequence of the secreted H chain of anti-PDL1
EVQLLESGGGLVQPGGSLRLSCAASGFTFSSYIMMWVRQAPGKGLEWVSSIYPSGGITF
YADTVKGRFTISRDNSKNTLYLQMNSLRAEDTAVYYCARIKLGTVTTVDYWGQGTLV
TVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCPPCPA
PELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 3
Peptide sequence of the secreted H chain of anti-PDL1/TGFβ Trap
EVQLLESGGGLVQPGGSLRLSCAASGFTFSSYIMMWVRQAPGKGLEWVSSIYPSGGITF
YADTVKGRFTISRDNSKNTLYLQMNSLRAEDTAVYYCARIKLGTVTTVDYWGQGTLV
TVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCPPCPA
PELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGAGGGGSGGGGSGGGGS
GGGGSGIPPHVQKSVNNDMIVTDNNGAVKFPQLCKFCDVRFSTCDNQKSCMSNCSITS
ICEKPQEVCVAVWRKNDENITLETVCHDPKLPYHDFILEDAASPKCIMKEKKKPGETFF
MCSCSSDECNDNIIFSEEYNTSNPD
SEQ ID NO: 4
DNA sequence from the translation initiation codon to the translation stop codon of the
anti-PD-L1 lambda light chain (the leader sequence preceding the VL is the signal peptide
from urokinase plasminogen activator)
atgagggccctgctggctagactgctgctgtgcgtgctggtcgtgtccgacagcaagggcCAGTCCGCCCTGACCCAG
CCTGCCTCCGTGTCTGGCTCCCCTGGCCAGTCCATCACCATCAGCTGCACCGGCAC
CTCCAGCGACGTGGGCGGCTACAACTACGTGTCCTGGTATCAGCAGCACCCCGGCA
AGGCCCCCAAGCTGATGATCTACGACGTGTCCAACCGGCCCTCCGGCGTGTCCAAC
AGATTCTCCGGCTCCAAGTCCGGCAACACCGCCTCCCTGACCATCAGCGGACTGCA
GGCAGAGGACGAGGCCGACTACTACTGCTCCTCCTACACCTCCTCCAGCACCAGAG
TGTTCGGCACCGGCACAAAAGTGACCGTGCTGggccagcccaaggccaacccaaccgtgacactgttcc
ccccatcctccgaggaactgcaggccaacaaggccaccctggtctgcctgatctcagatnctatccaggcgccgtgaccgtggcctgg
aaggctgatggctccccagtgaaggccggcgtggaaaccaccaagccctccaagcagtccaacaacaaatacgccgcctcctcctacc
tgtccctgacccccgagcagtggaagtcccaccggtcctacagctgccaggtcacacacgagggctccaccgtggaaaagaccgtcg
cccccaccgagtgctcaTGA
SEQ ID NO: 5
DNA sequence from the translation initiation codon to the translation stop codon (mVK SP
leader: small underlined; VH: capitals; IgG1m3 with K to A mutation: small letters;
(G4S)x4-G (SEQ ID NO: 11) linker: bold capital letters; TGFβRII: bold underlined small
letters; two stop codons: bold underlined capital letters)
atggaaacagacaccctgctgctgtgggtgctgctgctgtgggtgcccggctccacaggcGAGGTGCAGCTGCTGGAA
TCCGGCGGAGGACTGGTGCAGCCTGGCGGCTCCCTGAGACTGTCTTGCGCCGCCTC
CGGCTTCACCTTCTCCAGCTACATCATGATGTGGGTGCGACAGGCCCCTGGCAAGG
GCCTGGAATGGGTGTCCTCCATCTACCCCTCCGGCGGCATCACCTTCTACGCCGAC
ACCGTGAAGGGCCGGTTCACCATCTCCCGGGACAACTCCAAGAACACCCTGTACCT
GCAGATGAACTCCCTGCGGGCCGAGGACACCGCCGTGTACTACTGCGCCCGGATC
AAGCTGGGCACCGTGACCACCGTGGACTACTGGGGCCAGGGCACCCTGGTGACAG
TGTCCTCCgctagcaccaagggcccatcggtcttccccctggcaccctcctccaagagcacctctgggggcacagcggccctgg
gctgcctggtcaaggactacttccccgaaccggtgacggtgtcgtggaactcaggcgccctgaccagcggcgtgcacaccttcccggct
gtcctacagtcctcaggactctactccctcagcagcgtggtgaccgtgccctccagcagcttgggcacccagacctacatctgcaacgtg
aatcacaagcccagcaacaccaaggtggacaagagagttgagcccaaatcttgtgacaaaactcacacatgcccaccgtgcccagcac
ctgaactcctggggggaccgtcagtcttcctcttccccccaaaacccaaggacaccctcatgatctcccggacccctgaggtcacatgcg
tggtggtggacgtgagccacgaagaccctgaggtcaagttcaactggtacgtggacggcgtggaggtgcataatgccaagacaaagcc
gcgggaggagcagtacaacagcacgtaccgtgtggtcagcgtcctcaccgtcctgcaccaggactggctgaatggcaaggagtacaa
gtgcaaggtctccaacaaagccctcccagcccccatcgagaaaaccatctccaaagccaaagggcagccccgagaaccacaggtgta
caccctgcccccatcccgggaggagatgaccaagaaccaggtcagcctgacctgcctggtcaaaggcttctatcccagcgacatcgcc
gtggagtgggagagcaatgggcagccggagaacaactacaagaccacgcctcccgtgctggactccgacggctccucttcctctatag
caagctcaccgtggacaagagcaggtggcagcaggggaacgtcttctcatgctccgtgatgcatgaggctctgcacaaccactacacg
cagaagagcctctccctgtccccgggtgctGGCGGCGGAGGAAGCGGAGGAGGTGGCAGCGGTG
GCGGTGGCTCCGGCGGAGGTGGCTCCGGAatccctccccacgtgcagaagtccgtgaacaacgac
atgatcgtgaccgacaacaacggcgccgtgaagttccctcagctgtgcaagttctgcgacgtgaggttcagcacctgcgacaacc
agaagtectgcatgagcaactgcagcatcacaagcatctgcgagaagccccaggaggtgtgtgtggccgtgtggaggaagaac
gacgaaaacatcaccdcgagaccgtgtgccatgaccccaagctgccctaccacgacttcatcctggaagacgccgcctccccc
aagtgcatcatgaaggagaagaagaagcccggcgagaccttetcatgtgcagctgcagcagcgacgagtgcaatgacaacat
catctttagcgaggagtacaacaccagcaaccccgacTGATAA
SEQ ID NO: 6
Polypeptide sequence of the secreted lambda light chain of anti-PD-L1(mut)/TGFβ Trap,
with mutations A31G, D52E, R99Y
QSALTQPASVSGSPGQSITISCTGTSSDVGGYNYVSWYQQHPGKAPKLMIYEVSNRPSG
VSNRFSGSKSGNTASLTISGLQAEDEADYYCSSYTSSSTYVFGTGTKVTVLGQPKANPT
VTLFPPSSEELQANKATLVCLISDFYPGAVTVAWKADGSPVKAGVETTKPSKQSNNKY
AASSYLSLTPEQWKSHRSYSCQVTHEGSTVEKTVAPTECS
SEQ ID NO: 7
Polypeptide sequence of the secreted heavy chain of anti-PD-L1(mut)/TGFβ Trap
EVQLLESGGGLVQPGGSLRLSCAASGFTFSMYMMMWVRQAPGKGLEWVSSIYPSGGI
TFYADSVKGRFTISRDNSKNTLYLQMNSLRAEDTAIYYCARIKLGTVTTVDYWGQGTL
VTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCDKTHTCPPCP
APELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGAGGGGSGGGGSGGG
GSGGGGSGIPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIMKEKKKPGET
FFMCSCSSDECNDNIIFSEEYNTSNPD
SEQ ID NO: 8
Human TGFβRII Isoform A Precursor Polypeptide (NCBI RefSeq Accession No: NP_001020018)
MGRGLLRGLWPLHIVLWTRIASTIPPHVQKSDVEMEAQKDEIICPSCNRTAHPLRHINN
DMIVTDNNGAVKFPQLCKFCDVRFSTCDNQKSCMSNCSITSICEKPQEVCVAVWRKN
DENITLETVCHDPKLPYHDFILEDAASPKCIMKEKKKPGETFFMCSCSSDECNDNIIFSE
EYNTSNPDLLLVIFQVTGISLLPPLGVAISVIIIFYCYRVNRQQKLSSTWETGKTRKLMEF
SEHCAIILEDDRSDISSTCANNINHNTELLPIELDTLVGKGRFAEVYKAKLKQNTSEQFE
TVAVKIFPYEEYASWKTEKDIFSDINLKHENILQFLTAEERKTELGKQYWLITAFHAKG
NLQEYLTRHVISWEDLRKLGSSLARGIAHLHSDHTPCGRPKMPIVHRDLKSSNILVKND
LTCCLCDFGLSLRLDPTLSVDDLANSGQVGTARYMAPEVLESRMNLENVESFKQTDV
YSMALVLWEMTSRCNAVGEVKDYEPPFGSKVREHPCVESMKDNVLRDRGRPEIPSFW
LNHQGIQMVCETLTECWDHDPEARLTAQCVAERFSELEHLDRLSGRSCSEEKIPEDGSL
NTTK
SEQ ID NO: 9
Human TGFβRII Isoform B Precursor Polypeptide (NCBI RefSeq Accession No: NP_003233
MGRGLLRGLWPLHIVLWTRIASTIPPHVQKSVNNDMIVTDNNGAVKFPQLCKFCDVRF
STCDNQKSCMSNCSITSICEKPQEVCVAVWRKNDENITLETVCHDPKLPYHDFILEDAA
SPKCIMKEKKKPGETFFMCSCSSDECNDNIIFSEEYNTSNPDLLLVIFQVTGISLLPPLGV
AISVIIIFYCYRVNRQQKLSSTWETGKTRKLMEFSEHCAIILEDDRSDISSTCANNINHNT
ELLPIELDTLVGKGRFAEVYKAKLKQNTSEQFETVAVKIFPYEEYASWKTEKDIFSDIN
LKHENILQFLTAEERKTELGKQYWLITAFHAKGNLQEYLTRHVISWEDLRKLGSSLAR
GIAHLHSDHTPCGRPKMPIVHRDLKSSNILVKNDLTCCLCDFGLSLRLDPTLSVDDLAN
SGQVGTARYMAPEVLESRMNLENVESFKQTDVYSMALVLWEMTSRCNAVGEVKDYE
PPFGSKVREHPCVESMKDNVLRDRGRPEIPSFWLNHQGIQMVCETLTECWDHDPEARL
TAQCVAERFSELEHLDRLSGRSCSEEKIPEDGSLNTTK
SEQ ID NO: 10
A Human TGFβRII Isoform B Extracellular Domain Polypeptide
IPPHVQKSVNNDMIVTDNNGAVKFPQLCKFCDVRFSTCDNQKSCMSNCSITSICEKPQE
VCVAVWRKNDENITLETVCHDPKLPYHDFILEDAASPKCIMKEKKKPGETFFMCSCSS
DECNDNIIFSEEYNTSNPD
SEQ ID NO: 11
(Gly₄Ser)₄Gly linker
GGGGSGGGGSGGGGSGGGGSG
SEQ ID NO: 12
Polypeptide sequence of the secreted heavy chain variable region of anti-PD-L1 antibody
MPDL3289A
EVQLVESGGGLVQPGGSLRLSCAASGFTFSDSWIHWVRQAPGKGLEWVAWISPYGGS
TYY
ADSVKGRFTISADTSKNTAYLQMNSLRAEDTAVYYCARRHWPGGFDYWGQGTLVTV
SS
SEQ ID NO: 13
Polypeptide sequence of the secreted light chain variable region of anti-PD-L1 antibody
MPDL3289A
DIQMTQSPSSLSASVGDRVTITCRASQDVSTAVAWYQQKPGKAPKLLIYSASFLYSGVP
SRFSGSGSGTDFTLTISSLQPEDFATYYCQQYLYHPATFGQGTKVEIKR
SEQ ID NO: 14
Polypeptide sequence of the secreted heavy chain variable region of anti-PD-L1 antibody
YW243.55S70
EVQLVESGGGLVQPGGSLRLSCAASGFTFSDSWIHWVRQAPGKGLEWVAWISPYGGS
TYYADSVKGRFTISADTSKNTAYLQMNSLRAEDTAVYYCARRHWPGGFDYWGQGTL
VTVSA

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

The numbered embodiments of the present disclosure are listed below:
1. A method of treating cancer or inhibiting tumor growth in a subject in need thereof, the method comprising administering to the subject a dose of at least 500 mg of a protein comprising a first polypeptide and a second polypeptide,

wherein the first polypeptide comprises: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ),

wherein the second polypeptide comprises at least a variable region of a light chain of an antibody that binds PD-L1, and

wherein the heavy chain of the first polypeptide and the light chain of the second polypeptide, when combined, form an antigen binding site that binds PD-L1.
2. The method of claim 1, wherein the first polypeptide comprises the amino acid sequence of SEQ ID NO: 3, and the second polypeptide comprises the amino acid sequence of SEQ ID NO: 1.
3. The method of claim 1 or 2, wherein the dose is 500 mg to 2400 mg.
4. The method of claim 1 or 2, wherein the dose is 1200 mg to 1800 mg.
5. The method of any one of claims 1-4, wherein the dose is 1200 mg.
6. The method of claim 1 or 2, wherein the dose is 1800 mg.
7. The method of any one of claims 1-6, wherein the dose is administered once every two weeks or once every three weeks.
8. The method of any one of claims 1-7, wherein the protein is administered by intravenous administration.
9. The method of claim 8, wherein the intravenous administration is performed with a prefilled bag, a prefilled pen, or a prefilled syringe comprising a formulation comprising the protein.
10. The method of claim 9, wherein the bag is connected to a channel comprising a tube and/or a needle.
11. The method of any one of claims 1-10, wherein the cancer or tumor is selected from the group consisting of: non-small cell lung cancer, melanoma, pancreatic cancer, colorectal cancer, ovarian cancer, glioblastoma, gastric cancer, biliary tract cancer, esophageal cancer (squamous cell carcinoma or adenocarcinoma), adenoma of the head or the neck, and squamous carcinoma of the head or the neck.
12. The method of any one of claims 1-10, wherein the cancer or tumor is selected from the group consisting of: colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, non-small cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodysplastic syndromes.
13. The method of any one of claims 1-10, wherein the tumor is an advanced solid tumor.
14. The method of claim 13, wherein the tumor is refractory and/or resistant to prior treatment.
15. An intravenous drug delivery formulation comprising 500 mg-2400 mg of a protein comprising a first polypeptide and a second polypeptide,

wherein the first polypeptide comprises: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ),

wherein the second polypeptide comprises at least a variable region of a light chain of an antibody that binds PD-L1, and

wherein the heavy chain of the first polypeptide and the light chain of the second polypeptide, when combined, form an antigen binding site that binds PD-L1.
16. The intravenous drug delivery formulation of claim 15, wherein the first polypeptide comprises the amino acid sequence of SEQ ID NO: 3, and the second polypeptide comprises the amino acid sequence of SEQ ID NO: 1.
17. The intravenous drug delivery formulation of claim 15 or 16 comprising 1200 mg of the protein.
18. The intravenous drug delivery formulation of claim 15 or 16 comprising 1200 mg to 2400 mg of the protein.
19. The intravenous drug delivery formulation of claim 15 or 16 comprising 1800 mg of the protein.
20. The intravenous drug delivery formulation of any one of claims 15-19, wherein the formulation is contained in a bag, a pen, or a syringe.
21. The intravenous drug delivery formulation of claim 20, wherein the bag is connected to a channel comprising a tube and/or a needle.
22. The intravenous drug delivery formulation of any one of claims 15-21, wherein the formulation is a lyophilized formulation or a liquid formulation.
23. A drug delivery device comprising a formulation comprising 500 mg-2400 mg of a protein comprising a first polypeptide and a second polypeptide,

wherein the first polypeptide comprises: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ),

wherein the second polypeptide comprises at least a variable region of a light chain of an antibody that binds PD-L1, and

wherein the heavy chain of the first polypeptide and the light chain of the second polypeptide, when combined, form an antigen binding site that binds PD-L1.
24. The drug delivery device of claim 23, wherein the first polypeptide comprises the amino acid sequence of SEQ ID NO: 3, and the second polypeptide comprises the amino acid sequence of SEQ ID NO: 1.
25. The drug delivery device of claim 23 or 24 comprising 1200 mg of the protein.
26. The drug delivery device of claim 23 or 24 comprising 1200 mg to 2400 mg of the protein.
27. The drug delivery device of claim 23 or 24 comprising 1800 mg of the protein.
28. The drug delivery device of any one of claims 23-27, wherein the device is a bag, a pen, or a syringe.
29. The drug delivery device of claim 28, wherein the bag is connected to a channel comprising a tube and/or a needle.
30. A kit comprising one or more vessels collectively comprising a formulation comprising 500 mg-2400 mg of a protein comprising a first polypeptide and a second polypeptide,

wherein the first polypeptide comprises: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ),

wherein the second polypeptide comprises at least a variable region of a light chain of an antibody that binds PD-L1, and

wherein the heavy chain of the first polypeptide and the light chain of the second polypeptide, when combined, form an antigen binding site that binds PD-L1.
31. The kit of claim 30, wherein the first polypeptide comprises the amino acid sequence of SEQ ID NO: 3, and the second polypeptide comprises the amino acid sequence of SEQ ID NO: 1.
32. The kit of claim 30 or 31, wherein the vessels collectively comprise 1200 mg of the protein.
33. The kit of claim 30 or 31, wherein the vessels collectively comprise 1200 to 2400 mg of the protein.
34. The kit of claim 30 or 31, wherein the vessels collectively comprise 1800 mg of the protein.
35. The kit of any one of claims 30-34, wherein the formulation is a lyophilized formulation or a liquid formulation.
36. The intravenous drug delivery formulation of any one of claims 15-22, the drug delivery device of any one of claims 23-29, or the kit of any one of claims 30-35, for use in treating cancer or inhibiting tumor growth in a subject in need thereof.
37. The intravenous drug delivery formulation, the drug delivery device, or the kit of claim 36, wherein the cancer or tumor is selected from the group consisting of: non-small cell lung cancer, melanoma, pancreatic cancer, colorectal cancer, ovarian cancer, glioblastoma, gastric cancer, biliary tract cancer, esophageal cancer (squamous cell carcinoma or adenocarcinoma), adenoma of the head or the neck, and squamous carcinoma of the head or the neck
38. The intravenous drug delivery formulation, the drug delivery device, or the kit of claim 36, wherein the cancer or tumor is selected from the group consisting of: colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, non-small cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodysplastic syndromes.
39. The intravenous drug delivery formulation, the drug delivery device, or the kit of claim 36, wherein the tumor is an advanced solid tumor.
40. The intravenous drug delivery formulation, the drug delivery device, or the kit of claim 36, wherein the tumor is refractory to prior treatment.
41. The intravenous drug delivery formulation, the drug delivery device, or the kit of any one of claims 36-40, wherein the formulation is administered to the subject once every two weeks.

What is claimed is:

1. A method of treating cancer or inhibiting tumor growth in a subject in need thereof, the method comprising administering to the subject a dose of at least 500 mg of a protein comprising a first polypeptide and a second polypeptide,
wherein the first polypeptide comprises: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ),
wherein the second polypeptide comprises at least a variable region of a light chain of an antibody that binds PD-L1, and
wherein the heavy chain of the first polypeptide and the light chain of the second polypeptide, when combined, form an antigen binding site that binds PD-L1.

2. The method of claim 1, wherein the first polypeptide comprises the amino acid sequence of SEQ ID NO: 3, and the second polypeptide comprises the amino acid sequence of SEQ ID NO: 1.

3. The method of claim 1 or 2, wherein the dose is 500 mg to 2400 mg.

4. The method of claim 1 or 2, wherein the dose is 1200 mg to 1800 mg.

5. The method of any one of claims 1-4, wherein the dose is 1200 mg.

6. The method of claim 1 or 2, wherein the dose is 1800 mg.

7. The method of any one of claims 1-6, wherein the dose is administered once every two weeks or once every three weeks.

8. The method of any one of claims 1-7, wherein the protein is administered by intravenous administration.

9. The method of claim 8, wherein the intravenous administration is performed with a prefilled bag, a prefilled pen, or a prefilled syringe comprising a formulation comprising the protein.

10. The method of claim 9, wherein the bag is connected to a channel comprising a tube and/or a needle.

11. The method of any one of claims 1-10, wherein the cancer or tumor is selected from the group consisting of: non-small cell lung cancer, melanoma, pancreatic cancer, colorectal cancer, ovarian cancer, glioblastoma, gastric cancer, biliary tract cancer, esophageal cancer (squamous cell carcinoma or adenocarcinoma), adenoma of the head or the neck, and squamous carcinoma of the head or the neck.

12. The method of any one of claims 1-10, wherein the cancer or tumor is selected from the group consisting of: colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, non-small cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodysplastic syndromes.

13. The method of any one of claims 1-10, wherein the tumor is an advanced solid tumor.

14. The method of claim 13, wherein the tumor is refractory and/or resistant to prior treatment.

15. An intravenous drug delivery formulation comprising 500 mg-2400 mg of a protein comprising a first polypeptide and a second polypeptide,
wherein the first polypeptide comprises: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ),
wherein the second polypeptide comprises at least a variable region of a light chain of an antibody that binds PD-L1, and
wherein the heavy chain of the first polypeptide and the light chain of the second polypeptide, when combined, form an antigen binding site that binds PD-L1.

16. The intravenous drug delivery formulation of claim 15, wherein the first polypeptide comprises the amino acid sequence of SEQ ID NO: 3, and the second polypeptide comprises the amino acid sequence of SEQ ID NO: 1.

17. The intravenous drug delivery formulation of claim 15 or 16 comprising 1200 mg of the protein.

18. The intravenous drug delivery formulation of claim 15 or 16 comprising 1200 mg to 2400 mg of the protein.

19. The intravenous drug delivery formulation of claim 15 or 16 comprising 1800 mg of the protein.

20. The intravenous drug delivery formulation of any one of claims 15-19, wherein the formulation is contained in a bag, a pen, or a syringe.

21. The intravenous drug delivery formulation of claim 20, wherein the bag is connected to a channel comprising a tube and/or a needle.

22. The intravenous drug delivery formulation of any one of claims 15-21, wherein the formulation is a lyophilized formulation or a liquid formulation.

23. A drug delivery device comprising a formulation comprising 500 mg-2400 mg of a protein comprising a first polypeptide and a second polypeptide,
wherein the first polypeptide comprises: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ),
wherein the second polypeptide comprises at least a variable region of a light chain of an antibody that binds PD-L1, and
wherein the heavy chain of the first polypeptide and the light chain of the second polypeptide, when combined, form an antigen binding site that binds PD-L1.

24. The drug delivery device of claim 23, wherein the first polypeptide comprises the amino acid sequence of SEQ ID NO: 3, and the second polypeptide comprises the amino acid sequence of SEQ ID NO: 1.

25. The drug delivery device of claim 23 or 24 comprising 1200 mg of the protein.

26. The drug delivery device of claim 23 or 24 comprising 1200 mg to 2400 mg of the protein.

27. The drug delivery device of claim 23 or 24 comprising 1800 mg of the protein.

28. The drug delivery device of any one of claims 23-27, wherein the device is a bag, a pen, or a syringe.

29. The drug delivery device of claim 28, wherein the bag is connected to a channel comprising a tube and/or a needle.

30. A kit comprising one or more vessels collectively comprising a formulation comprising 500 mg-2400 mg of a protein comprising a first polypeptide and a second polypeptide,
wherein the first polypeptide comprises: (a) at least a variable region of a heavy chain of an antibody that binds to human protein Programmed Death Ligand 1 (PD-L1); and (b) human Transforming Growth Factor β Receptor II (TGFβRII), or a fragment thereof, capable of binding Transforming Growth Factor β (TGFβ),
wherein the second polypeptide comprises at least a variable region of a light chain of an antibody that binds PD-L1, and
wherein the heavy chain of the first polypeptide and the light chain of the second polypeptide, when combined, form an antigen binding site that binds PD-L1.

31. The kit of claim 30, wherein the first polypeptide comprises the amino acid sequence of SEQ ID NO: 3, and the second polypeptide comprises the amino acid sequence of SEQ ID NO: 1.

32. The kit of claim 30 or 31, wherein the vessels collectively comprise 1200 mg of the protein.

33. The kit of claim 30 or 31, wherein the vessels collectively comprise 1200 to 2400 mg of the protein.

34. The kit of claim 30 or 31, wherein the vessels collectively comprise 1800 mg of the protein.

35. The kit of any one of claims 30-34, wherein the formulation is a lyophilized formulation or a liquid formulation.

36. The intravenous drug delivery formulation of any one of claims 15-22, the drug delivery device of any one of claims 23-29, or the kit of any one of claims 30-35, for use in treating cancer or inhibiting tumor growth in a subject in need thereof.

37. The intravenous drug delivery formulation, the drug delivery device, or the kit of claim 36, wherein the cancer or tumor is selected from the group consisting of: non-small cell lung cancer, melanoma, pancreatic cancer, colorectal cancer, ovarian cancer, glioblastoma, gastric cancer, biliary tract cancer, esophageal cancer (squamous cell carcinoma or adenocarcinoma), adenoma of the head or the neck, and squamous carcinoma of the head or the neck

38. The intravenous drug delivery formulation, the drug delivery device, or the kit of claim 36, wherein the cancer or tumor is selected from the group consisting of: colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, non-small cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodysplastic syndromes.

39. The intravenous drug delivery formulation, the drug delivery device, or the kit of claim 36, wherein the tumor is an advanced solid tumor.

40. The intravenous drug delivery formulation, the drug delivery device, or the kit of claim 36, wherein the tumor is refractory to prior treatment.

41. The intravenous drug delivery formulation, the drug delivery device, or the kit of any one of claims 36-40, wherein the formulation is administered to the subject once every two weeks.
